SEQUENCE LISTING (103 sequences)

SEQ ID NO: 1 - DNA, 300 bp, Clostridium autoethanogenum
atgcaatggttaaggcagctaacgttaacttaataggtaaagaacatgttggcggcggtcttgtaacagtaatggtaagaggagatgtaggagcagtaaaggcagctactgatgctggtgctgcagcagcacaacgtgttggagaattagtttctgttcatgttattcctcgtccacacgttgaagttgaaactatccttcctaaaactggtttaaaggaagactaatattagaagatataggatataagatacaaagtaaaaataaaaaaaataaattataaactaggaggaaataaaa

SEQ ID NO: 2 - DNA, 301 bp, Clostridium autoethanogenum
atgcaatggttaaggcagctaacgttaacttaataggtaaagaacatgttggcggcggtcttgtaacagtaatggtaagaggagatgtaggagcagtaaaggcagctactgatgctggtgctgcagcagcacaacgtgttggagaattagtttctgttcatgttattcctcgtccacacgttgaagttgaaactatccttcctaaaactggtttaaaggaagactaatattagaagatataggatataagatacaaagtaaaaaataaaaaaaataaattataaactaggaggaaataaaa

SEQ ID NO: 3 - DNA, 297 bp, Clostridium autoethanogenum
atgaaatatgatgcattaggaatgattgaaacaaaaggtttagtaggttcaattgaagctgcagatgcaatggtaaaggcagctaatgtttatctaataggaaaagaacatgttggtggcggtcttgtaacagtaatggtaagaggagatgtaggagctgtaaaggcagctactgatgctggtgctgcagcagcacaacgtgttggagaattagtttctgttcatgttattcctcgtccacacgttgaagttgaaactatccttcctaaaactaatttaaaggaagatgaaaagtag

SEQ ID NO: 4 - protein, 98 aa, Clostridium autoethanogenum
MKYDALGMIETKGLVGSIEAADAMVKAANVYLIGKEHVGGGLVTVMVRGDVGAVKAATDAGAAAAQRVGELVSVHVIPRPHVEVETILPKTNLKEDEK

SEQ ID NO: 5 - DNA, 1290 bp, Clostridium autoethanogenum
atggcagttataattacagattcttgtattggatgtgaatcatgtatcccaatttgccccttcgatgcattggggattaatggagaaggtaaattagtggctagtaaagaaaaatgtactgaatgtggaaagtgtgtatcggtgtgtccagtttctgcgttaaatatttcaggtgtcaacaagaaagccaatgatatctcgaagaatgagactaaggaaggtagcaaagcttcaaagtcagaaaaacctactggagatgtatgggtctttgcagaacaattagaaggtaagctatcttctgttactcttgaacttattggtgctgcaagaaagcttgcttcaaaattagaggtaaaggtatgtacagttttattaggtgataaagttgaatctataatacctgagctttttggatatggtgcagataccatatatgttatagatgatgaagtatttcatttttatagagcagagacttataatagagctttctgctatttaataaataagtataaaccagaaatattattaatgggagcaacaactacgggaagagatttagctggtgccgttgcaactgcattgaaaactggacttacagcagactgtaccggactagatattgacttagaaaaaagagcattgcttgctagccgtccagcctttggaggaaatataatggcaacaatagtatgtgagaaacaccgtcctcaaatggctactgtgcgtccaagagttatgatgatgccagaacctgaagtaaataaaacaggcgctataattagagagaattttaaaattgaagaaaaatgtcttaagacttgggtacttgagattattaaggaacaggcagaaaatgcaaaacttgaagatgctaaaattatagtttgtggtggacgtggtgtgcagaatcaggaaggatttaagttgctagaggagttagcaaaggttgttggaggtgtagttgcaggaagtcgcggcgctgttgagaaaggtttagtagatcataaacgtcaagttggtcagacaggacaaacagtatccccaaaactatactttgctataggaatttctggtgcaatacagcataccgttggaatgcaaggagctgaaacaatagtttgtataaatacagattctgaatgtgaaatgatgaagttggctacttatggaatagagggagatgtttttgaagtattacctaaattaattagtagttttaaagaagaaattacagaagtttcgaagataaataataaaggtttgtgtacgattgcgaaggaggtttaa

SEQ ID NO: 6 - DNA, 1290 bp, Clostridium autoethanogenum
atggcagttataattacagattcttgtattggatgtgaatcatgtatcccaatttgccccttcgatgcattggggattaatggagaaggtaaattagtggctagtaaagaaaaatgtactgaatgtggaaagtgtgtatcggtgtgtccagtttctgcgttaaatatttcaggtgtcaacaagaaagccaatgatatctcgaagaatgagactaaggaaggtagcaaagcttcaaagtcagaaaaacctactggagatgtatgggtctttgcagaacaattagaaggtaagctatcttctgttactcttgaacttattggtgctgcaagaaagcttgcttcaaaattagaggtaaaggtatgtacagttttattaggtgataaagttgaatctataatacctgagctttttggatatggtgcagataccatatatgttatagatgatgaagtatttcatttttatagagcagagacttataatagagctttctgctatttaataaataagtataaaccagaaatattattaatgggagcaacaactacgggaagagatttagctggtgccgttgcaactgcattgaaaactggacttacagcagactgtaccggactagatattgacttagaaaaaagagtattgcttgctagccgtccagcctttggaggaaatataatggcaacaatagtatgtgagaaacaccgtcctcaaatggctactgtgcgtccaagagttacgatgatgccagaacctgaagtaaataaaacaggcgctataattagagagaattttaaaattgaagaaaaatgtcttaagacttgggtacttgagattattaaggaacaggcagaaaatgcaaaacttgaagatgctaaaattatagtttgtggtggacgtggtgtgcagaatcaggaaggatttaagttgctagaggagttagcaaaggttgttggaggtgtagttgcaggaagtcgcggcgctgttgagaaaggtttagtagatcataaacgtcaagttggtcagacaggacaaacagtatccccaaaactatactttgctataggaatttctggtgcaatacagcataccgttggaatgcaaggagctgaaacaatagtttgtataaatacagattctgaatgtgaaatgatgaagttggctacttatggaatagagggagatgtttttgaagtattacctaaattaattagtagttttaaagaagaaattacagaagtttcgaagataaataataaaggtttgtgtacgattgcgaaggaggtttaa

SEQ ID NO: 7 - protein, 429 aa, Clostridium autoethanogenum
MAVIITDSCIGCESCIPICPFDALGINGEGKLVASKEKCTECGKCVSVCPVSALNISGVNKKANDISKNETKEGSKASKSEKPTGDVWVFAEQLEGKLSSVTLELIGAARKLASKLEVKVCTVLLGDKVESIIPELFGYGADTIYVIDDEVFHFYRAETYNRAFCYLINKYKPEILLMGATTTGRDLAGAVATALKTGLTADCTGLDIDLEKRALLASRPAFGGNIMATIVCEKHRPQMATVRPRVMMMPEPEVNKTGAIIRENFKIEEKCLKTWVLEIIKEQAENAKLEDAKIIVCGGRGVQNQEGFKLLEELAKVVGGVVAGSRGAVEKGLVDHKRQVGQTGQTVSPKLYFAIGISGAIQHTVGMQGAETIVCINTDSECEMMKLATYGIEGDVFEVLPKLISSFKEEITEVSKINNKGLCTIAKEV

SEQ ID NO: 8 - protein, 429 aa, Clostridium autoethanogenum
MAVIITDSCIGCESCIPICPFDALGINGEGKLVASKEKCTECGKCVSVCPVSALNISGVNKKANDISKNETKEGSKASKSEKPTGDVWVFAEQLEGKLSSVTLELIGAARKLASKLEVKVCTVLLGDKVESIIPELFGYGADTIYVIDDEVFHFYRAETYNRAFCYLINKYKPEILLMGATTTGRDLAGAVATALKTGLTADCTGLDIDLEKRVLLASRPAFGGNIMATIVCEKHRPQMATVRPRVTMMPEPEVNKTGAIIRENFKIEEKCLKTWVLEIIKEQAENAKLEDAKIIVCGGRGVQNQEGFKLLEELAKVVGGVVAGSRGAVEKGLVDHKRQVGQTGQTVSPKLYFAIGISGAIQHTVGMQGAETIVCINTDSECEMMKLATYGIEGDVFEVLPKLISSFKEEITEVSKINNKGLCTIAKEV

SEQ ID NO: 9 - DNA, 810 bp, Clostridium autoethanogenum
ttgcatattttagtttgtgtgaaacaagtaccggatactacagaaattaaaatggatccaaaaacaaatactttagatagatcaagcgcacctacaattattaatccttatgatgcacatgcagtagaagaagctgtgaaaattaaaaataagtttggaggtaaggtttctattatatcaatggggccgccacaagcagaggaagttattaaaaagtgtattgaaatgggagctgatgaagggtatcttctctcagatagagcatttgctggttctgatactctagctacaagttatatactttcaatgggaataaaaaaaataatggagaaagaggctattgatttagtgttttgtggaaagcaagctatagatggagatacagctcaggttggaccaggtattgcatctagacttggaataccgcaattaacctatgtggaaaaaataaagtctttaaatttaaaaaataatactgtgataatacacagaaaaattgataatggctatgaagttgttgaatcaaaacttccatgcttaatcacagtagaaaaagatataaatgatttaagtttttctccactatcaaatattattagagcagcacgttataaagcaacagtttggggaattaatgattttgaagcagatatgagtcaattagggcttaaaggatctccaacttcagttcgacgtatatttccaccacctcaaaggtccggtggggagttactaaaaggcaacattgatgaggtggtaaaacaacttacagatagcttgaaaggaggaatagttagatggcagttataa

SEQ ID NO: 10 - DNA, 810 bp, Clostridium autoethanogenum
ttgcatattttagtttgtgtgaaacaagtaccggatactacagaaattaaaatggatccaaaaacaaatactttagatagatcaagcgcacctacaattattaatccttatgatgcacatgcagtagaagaagctgtgaaaattaaaaataagtttggaggtaagatttctattatatcaatggggccgccacaagcagaggaagttattaaaaagtgtattgaaatgggagctgatgaagggtatcttctctcagatagagcatttgctggttctgatactctagctacaagttatatactttcaatgggaataaaaaaaataatggagaaagaggctattgatttagtgttttgtggaaagcaagctatagatggagatacagctcaggttggaccaggtattgcatctagacttggaataccgcaattaacctatgtggaaaaaataaagtctttaaatttaaaaaataatactgtgataatacacagaaaaattgataatggctatgaagttgttgaatcaaaacttccatgcttaatcacagtagaaaaagatataaatgatttaagtttttctccactatcaaatattattagagcagcacgttataaagcaacagtttggggaattaatgattttgaagcagatatgagtcaattagggcttaaaggatctccaacttcagttcgacgtatatttccaccacctcaaaggtccggtggggagttactaaaaggcaacattgatgaggtggtaaaacaacttacagatagcttgaaaggaggaatagttagatggcagttataa

SEQ ID NO: 11 - protein, 269 aa, Clostridium autoethanogenum
MHILVCVKQVPDTTEIKMDPKTNTLDRSSAPTIINPYDAHAVEEAVKIKNKFGGKVSIISMGPPQAEEVIKKCIEMGADEGYLLSDRAFAGSDTLATSYILSMGIKKIMEKEAIDLVFCGKQAIDGDTAQVGPGIASRLGIPQLTYVEKIKSLNLKNNTVIIHRKIDNGYEVVESKLPCLITVEKDINDLSFSPLSNIIRAARYKATVWGINDFEADMSQLGLKGSPTSVRRIFPPPQRSGGELLKGNIDEVVKQLTDSLKGGIVRWQL

SEQ ID NO: 12 - protein, 269 aa, Clostridium autoethanogenum
MHILVCVKQVPDTTEIKMDPKTNTLDRSSAPTIINPYDAHAVEEAVKIKNKFGGKISIISMGPPQAEEVIKKCIEMGADEGYLLSDRAFAGSDTLATSYILSMGIKKIMEKEAIDLVFCGKQAIDGDTAQVGPGIASRLGIPQLTYVEKIKSLNLKNNTVIIHRKIDNGYEVVESKLPCLITVEKDINDLSFSPLSNIIRAARYKATVWGINDFEADMSQLGLKGSPTSVRRIFPPPQRSGGELLKGNIDEVVKQLTDSLKGGIVRWQL

SEQ ID NO: 13 - DNA, 249 bp, Clostridium autoethanogenum
atgagattagataaataccttaaagtatctagaataataaaaagaagaactgtagcaaaagaagcatgtgaaggtggaagggttgctataaatggaaaagtggccaaacctagtgttgaagtcaaagaaggggatataatagaaatcaaatatgccaataagtgtttaaaggcagaaatcttaaatattgcagctcatgttacaaaagaaaacgttcaaagtatgtataggataatttcaactgaataa

SEQ ID NO: 14 - DNA, 249 bp, Clostridium autoethanogenum
atgagattagataaataccttaaagtatctagaataataaaaagaagaactgtagcaaaagaagcatgtgaaggtggaagggttgctataaatggaaaagtggccaaacctagtgttgaagtcaaagaaggagatataatagaaatcaaatatgccaataagtgtttaaaggcagaaatcttaaatattgcagctcatgttacaaaagaaaacgttcaaagtatgtataggataatttcaactgaataa

SEQ ID NO: 15 - protein, 82 aa, Clostridium autoethanogenum
MRLDKYLKVSRIIKRRTVAKEACEGGRVAINGKVAKPSVEVKEGDIIEIKYANKCLKAEILNIAAHVTKENVQSMYRIISTE

SEQ ID NO: 16 - DNA, 400 bp, Clostridium autoethanogenum
ctacttctatttattaaatcatacatctcttattttaatataaatactctcttatatccatatagattatcaatattaaatttcttatcgaaaatttcgactttaaatatttaattatccatttagttactattttataaaaaatattttttcaaatttcctgattttgtacttaattttaatattaatttcttacatttaaatccttgtttatcatatattataaacttttattaatattgttattgacttttgaatattatggtagtatattaactttagatggaatttgattagaaatttgtaaaatgttttatgaaattatattgacttttaatgtaatttttgctattatctaattatagtcgtgtcgaattatggaattagaggaggaaaatca

SEQ ID NO: 17 - DNA, 400 bp, Clostridium autoethanogenum
ctacttctatttattaaatcatacatctcttattttaatataaatactctcttatatccatatagattatcaatattaattttcttatcgaaaatttcgactttaaatatttaattatccatttagttactattttataaaaaatattttttcaaatttcctgattttgtacttaattttaatattaatttcttacatttaaatccttgtttatcatatattataaacttttattaatattgttattgacttttgaatattatggtagtatattaactttagatggaatttgattagaaatttgtaaaatgttttatgaaattatattgacttttaatgtaatttttgctattatctaattatagtcgtgtcgaattatggaattagaggaggaaaatca

SEQ ID NO: 18 - DNA, 246 bp, Clostridium autoethanogenum
atgaaatcaacaggtgttgtaagaagagtagacgagctaggaagaattgtcattcctatagaattgaggagaactttagacattgctgaaaaagatgccttagaaatatatgtagatggtgaacaaattatattaaagaagtatgagcctgcatgtatcttctgcggtgacgcaagagacgttgtaaattatagaggtaagaacatctgcaaaagttgtttaaatgaattaaaagaaggcaaataa

SEQ ID NO: 19 - protein, 81 aa, Clostridium autoethanogenum
MKSTGVVRRVDELGRIVIPIELRRTLDIAEKDALEIYVDGEQIILKKYEPACIFCGDARDVVNYRGKNICKSCLNELKEGK

SEQ ID NO: 20 - DNA, 996 bp, Clostridium autoethanogenum
ttgcagatgaaaatgcaggaacctaatttctgggatcacttggaaagggcacaggaagtaacttctgaggaaaagtttttaaatgaaaagctggatatgtataataagttggagagtagaataaatgatgcagaaattctagctcatattgcggaagaagaggaagacgtttctgactgcaaagatatattatctgaagtggaagatatcgaaaatataattgataaacttaaaatagaaatacttttatcaggagaatatgataaaaacaatgccatattgaatttacatgtgggagtaggtggcacagatgcccaagactggacggagatgcttttgaggatgtataccagatgggcagaaaagtctgggtataaagttgagactttagatattcttcctgcagatgatgctggaataaaaagtgtatcattaaggataataggggaatttgcttatgggtatttgaaagccgaaaagggtattcatagattggttagaatatcacctttcaatgctaatgggaaaaggcagacttcttttgcatctgtagaggttttgccggaacttaccaaagaccaagacatagatataaggccagaagatttaagggtagatacctataggtcttctggtgcaggcggacagcacgtaaataaaacggaatctgcagtcaggataactcatatacctacgggtatagttgtacagtgccagaatgaaagaagtcagcattataatagagagactgcaatgctcatgttaaaatcaaaacttgtggaacttaaagaaagagcacataaggaaaagatagaagatcttgcaggagaacttaaggacatgggatggggaagccagataagatcctatgtattccatccatatactcttgtaaaagatcatagaactggagtagagaatgggaatgtgtcttcagtaatggatggagaaattgataattttatattagcctatttaagacagcaggcaaagtaa

SEQ ID NO: 21 - DNA, 996 bp, Clostridium autoethanogenum
ttgcagatgaaaatgcaggaacctaatttctgggatcacttggaaagggcacaggaagtaacttctgaggaaaagtttttaaatgaaaagctggatatgtataataagttggagagtagaataaatgatgcagaaattctagctcatattgcggaagaagaggaagacgtttctgactgcaaagatatattatctgaagcggaagatatcgaaaatataattgataaacttaaaatagaaatacttttatcaggagaatatgataaaaacaatgccatattgaatttacatgtgggagtaggtggcacagatgcccaagactggacggagatgcttttgaggatgtataccagatgggcagaaaagtctgggtataaagttgagactttagatattcttcctgcagatgatgctggaataaaaagtgtatcattaaggataataggggaatttgcttatgggtatttgaaagccgaaaagggtattcatagattggttagaatatcacctttcaatgctaatgggaaaaggcagacttcttttgcatctgtagaggttttgccggaacttaccaaagaccaagacatagatataaggccagaagatttaagggtagatacctataggtcttctggtgcaggcggacagcacgtaaataaaacggaatctgcagtcaggataactcatatacctacgggtatagttgtacagtgccagaatgaaagaagtcagcattataatagagagactgcaatgctcatgttaaaatcaaaacttgtggaacttaaagaaagagcacataaggaaaagatagaagatcttgcaggagaacttaaggacatgggatggggaagccagataagatcctatgtattccatccatatactcttgtaaaagatcatagaactggagtagagaatgggaatgtgtcttcagtaatggatggagaaattgataattttatattagcctatttaagacagcaggcaaagtaa

SEQ ID NO: 22 - protein, 331 aa, Clostridium autoethanogenum
MQMKMQEPNFWDHLERAQEVTSEEKFLNEKLDMYNKLESRINDAEILAHIAEEEEDVSDCKDILSEVEDIENIIDKLKIEILLSGEYDKNNAILNLHVGVGGTDAQDWTEMLLRMYTRWAEKSGYKVETLDILPADDAGIKSVSLRIIGEFAYGYLKAEKGIHRLVRISPFNANGKRQTSFASVEVLPELTKDQDIDIRPEDLRVDTYRSSGAGGQHVNKTESAVRITHIPTGIVVQCQNERSQHYNRETAMLMLKSKLVELKERAHKEKIEDLAGELKDMGWGSQIRSYVFHPYTLVKDHRTGVENGNVSSVMDGEIDNFILAYLRQQAK

SEQ ID NO: 23 - protein, 331 aa, Clostridium autoethanogenum
MQMKMQEPNFWDHLERAQEVTSEEKFLNEKLDMYNKLESRINDAEILAHIAEEEEDVSDCKDILSEAEDIENIIDKLKIEILLSGEYDKNNAILNLHVGVGGTDAQDWTEMLLRMYTRWAEKSGYKVETLDILPADDAGIKSVSLRIIGEFAYGYLKAEKGIHRLVRISPFNANGKRQTSFASVEVLPELTKDQDIDIRPEDLRVDTYRSSGAGGQHVNKTESAVRITHIPTGIVVQCQNERSQHYNRETAMLMLKSKLVELKERAHKEKIEDLAGELKDMGWGSQIRSYVFHPYTLVKDHRTGVENGNVSSVMDGEIDNFILAYLRQQAK

SEQ ID NO: 24 - DNA, 936 bp, Clostridium autoethanogenum
atggctaaaatagaattaccataccataaaaaaataatagaagctgaaatagatgacaacaacttacttggcatacttgaatcaaaagcaagcgaatataaagcagagctaggacaattggaaatagtagaaaaagctcttgacaatcctataaactcacctaaacttgaacaattggctgagaataaaaaaagtatagttataataagcagcgatcatacaagacctgttcctagcaaaattattatgcctgcacttttgagaagaataaggagtataaattctgatgctaatattactatactaattgccacaggccttcatagggctactactaggcaggaactcatagataaatatggagaagatatagtaaaaaatgaaaagatagtagtacatgatgcacagaatgaagataatctggtaaaaatcggtacattgccatctggtggtgaacttatagtaaataaacttgcagttaaagcagatcttttaattgcagaggggtttatagaatcacattgttttgctggcttttcaggaggtagaaagagtgtgctgcctggaatagcctctgccaagaccatcctggcaaatcactgtgctgaatttgtagcacatccttctgcaagagttggaaatttaaaagataatcctgttcataatgacatgctatatgcagctaaaaaggcaggattggctttcataataaatgtagcactagatgaagataaaaagataataaatgcttttgcaggtgatacaaatgatgcccatgtaaaaggctgtgaatttgtacataatttatcaaaagtgagtaaattagtaggagacatctccatttctaccaacggaggatatccacttgaccaaaatatatatcaatctgttaaggggatggcagcagccgggccaattgcagggatggcggcgtaa

SEQ ID NO: 25 - DNA, 937 bp, Clostridium autoethanogenum
atggctaaaatagaattaccataccataaaaaaataatagaagctgaaatagatgacaacaacttacttggcatacttgaatcaaaagcaagcgaatataaagcagagctaggacaattggaaatagtagaaaaagctcttgacaatcctataaactcacctaaacttgaacaattggctgagaataaaaaaagtatagttataataagcagcgatcatacaagacctgttcctagcaaaattattatgcctgcacttttgagaagaataaggagtataaattctgatgctaatattactatactaattgccacaggccttcatagggctactactaggcaggaactcatagataaatatggagaagatatagtaaaaaatgaaaagatagtagtacatgatgcacagaatgaagataatctggtaaaaatcggtacattgccatctggtggtgaacttatagtaaataaacttgcagttaaagcagatcttttaattgcagaggggtttatagaatcacattgttttgctggcttttcaggaggtagaaagagtgtgctgcctggaatagcctctgccaagaccatcctggcaaatcactgtgctgaatttgtagcacatccttctgcaagagttggaaatttaaaagataatcctgttcataatgacatgctatatgcagctaaaaaggcaggattggctttcataataaatgtagcactagatgaagataaaaagataataaatgcttttgcaggtgatacaaatgatgcccatgtaaaaggctgtgaatttgtacataatttatcaaaagtgagtaaattagtaggagacatctccatttctaccaacggaggatatccacttgaccaaaatatatatcaatctgttaaggggatggcagcagccggggccaattgcagggatggcggcgtaa

SEQ ID NO: 26 - protein, 311 aa, Clostridium autoethanogenum
MAKIELPYHKKIIEAEIDDNNLLGILESKASEYKAELGQLEIVEKALDNPINSPKLEQLAENKKSIVIISSDHTRPVPSKIIMPALLRRIRSINSDANITILIATGLHRATTRQELIDKYGEDIVKNEKIVVHDAQNEDNLVKIGTLPSGGELIVNKLAVKADLLIAEGFIESHCFAGFSGGRKSVLPGIASAKTILANHCAEFVAHPSARVGNLKDNPVHNDMLYAAKKAGLAFIINVALDEDKKIINAFAGDTNDAHVKGCEFVHNLSKVSKLVGDISISTNGGYPLDQNIYQSVKGMAAAGPIAGMAA

SEQ ID NO: 27 - protein, 312 aa, Clostridium autoethanogenum
MAKIELPYHKKIIEAEIDDNNLLGILESKASEYKAELGQLEIVEKALDNPINSPKLEQLAENKKSIVIISSDHTRPVPSKIIMPALLRRIRSINSDANITILIATGLHRATTRQELIDKYGEDIVKNEKIVVHDAQNEDNLVKIGTLPSGGELIVNKLAVKADLLIAEGFIESHCFAGFSGGRKSVLPGIASAKTILANHCAEFVAHPSARVGNLKDNPVHNDMLYAAKKAGLAFIINVALDEDKKIINAFAGDTNDAHVKGCEFVHNLSKVSKLVGDISISTNGGYPLDQNIYQSVKGMAAAGANCRDGGV

SEQ ID NO: 28 - DNA, 1086 bp, Clostridium autoethanogenum
atggcaaaaggaggcaataaaatgaaaagtaagagtgcaaagctacagttagttaaaaaaatcatagaaaaaggtaaaaaaaatggcaccttaacttataaagaaataatggacgaattagaagaagtggacttaagtcctgagcaaatagaaaaaatatatgaagctcttgaatccatgggaatagaagtaactggtgatgaacatgaaacagaatcaaatgagaaaaacttagatttatcggttcctgatggaattgcaatagatgatcctgtaagaatgtatttaaaagaaataggaaaagtaccgctgcttttaccagaagaagaaatatctcttgcaaaaagaatagaagagggagaccaaattgccaagaaaaagttagcagaagctaatttaagattagtggttagcatagctaaaagatatgtgggtagaggaatgcttttcttagatttaattcaagaaggtaaccttggacttataaaagcagtagaaaagtttgattacagaaagggatataaatttagtacctatgctacatggtggataaggcaggcaattacaagggcaatagcagatcaggcaagaacaataagaatacctgttcacatggtagaaaccataaataaacttgtaagagtttcaagacagttacttcaagaacttggaagagaacctcatccagaagaagtagcacaaataatggagatgcctgtagataaagtaagggagattatgaaaatagcacaggagcctgtatctcttgaaacccctattggagaagaggaagacagtcaccttggagattttataccagatgatgaggcaccagcacctgcagaggcagcagcttttactatgcttaaggagcaacttataaatgtattagataccttaactccaagggaagaaaaagttttaagacttaggtttggtctagatgacggaagagctagaactctagaagaagttggaaaagagtttaatgtaactagagagagaataaggcagatagaggccaaggcgcttagaaagctgaggcatccaagtagaagtaaaaaattaaaagattatttagattaa

SEQ ID NO: 29 - DNA, 1086 bp, Clostridium autoethanogenum
atggcaaaaggaggcaataaaatgaaaagtaagagcgcaaagctacagttagttaaaaaaatcatagaaaaaggtaaaaaaaatggcaccttaacttataaagaaataatggacgaattagaagaagtggacttaagtcctgagcaaatagaaaaaatatatgaagctcttgaatccatgggaatagaagtaactggtgatgaacatgaaacagaatcaaatgagaaaaacttagatttatcggttcctgatggaattgcaatagatgatcctgtaagaatgtatttaaaagaaataggaaaagtaccgctgcttttaccagaagaagaaatatctcttgcaaaaagaatagaagagggagaccaaattgccaagaaaaagttagcagaagctaatttaagattagtggttagcatagctaaaagatatgtgggtagaggaatgcttttcttagatttaattcaagaaggtaaccttggacttataaaagcagtagaaaagtttgattacagaaagggatataaatttagtacctatgctacatggtggataaggcaggcaattacaagggcaatagcagatcaggcaagaacaataagaatacctgttcacatggtagaaaccataaataaacttgtaagagtttcaagacagttacttcaagaacttggaagagaacctcatccagaagaagtagcacaaataatggagatgcctgtagataaagtaagggagattatgaaaatagcacaggagcctgtatctcttgaaacccctattggagaagaggaagacagtcaccttggagattttataccagatgatgaggcaccagcacctgcagaggcagcagcttttactatgcttaaggagcaacttataaatgtattagataccttaactccaagggaagaaaaagttttaagacttaggtttggtctagatgacggaagagctagaactctagaagaagttggaaaagagtttaatgtaactagagagagaataaggcagatagaggccaaggcgcttagaaagctgaggcatccaagtagaagtaaaaaattaaaagattatttagattaa

SEQ ID NO: 30 - protein, 361 aa, Clostridium autoethanogenum
MAKGGNKMKSKSAKLQLVKKIIEKGKKNGTLTYKEIMDELEEVDLSPEQIEKIYEALESMGIEVTGDEHETESNEKNLDLSVPDGIAIDDPVRMYLKEIGKVPLLLPEEEISLAKRIEEGDQIAKKKLAEANLRLVVSIAKRYVGRGMLFLDLIQEGNLGLIKAVEKFDYRKGYKFSTYATWWIRQAITRAIADQARTIRIPVHMVETINKLVRVSRQLLQELGREPHPEEVAQIMEMPVDKVREIMKIAQEPVSLETPIGEEEDSHLGDFIPDDEAPAPAEAAAFTMLKEQLINVLDTLTPREEKVLRLRFGLDDGRARTLEEVGKEFNVTRERIRQIEAKALRKLRHPSRSKKLKDYLD

SEQ ID NO: 31 - DNA, 2214 bp, Clostridium autoethanogenum
atgttatctaaaaaagttataactactttaataatttctacagcattaatattttcaaaaactactgtaatcaatgctcagtctcaaagtccaactataacaagattatctggcccgaacagatactcaactaatatgaaaatagctgactatggctggagcggtcattctgattatgcaattattgtttcaggtgaaaactttccagatgcaatttgtgctgcacctttaagtgccaagtacaatgcacctttattaatgacagatggaaacactttaagtcctgaaatacaaaataaattgacaagcctaggcattaaaaatatatttttaattggtgggacaggcgctgtttcaagtgaaatagaaaatgaattaacccaaaaaaatttaaatgtaaaaagattgggtggatctacaagaattgaaacttccattgaaattgcaaaagaagttggaggtaatcagctatttgttgtttcatcagaaagttttgcagatgcattatccatttcatcctatgcttcaagtaaagctgaacctattgttttaactcctaaaagtgatattcctgatgtacttaaagactatttaagtactttaaaaccttcaaaaacctatgtgattggtggacaaggtgttattcctgacgataccgcttcttactttggtgattatgaaagactaagtggtcaaagcagatatgatacaaatgcaaatgtaattaaagaattttatgatctttcaaaagtacaaaaaatatacgtagcagcaggaataaactatccagatggactatctatttctcctctagctgccaaaaataattcacctgtaatactcgtaaatgacgatatgtcttccgaccagattagaatggttagaaacaataaagctacactaaaagagtgtgtggttgtaggtggagaagatgttactccagactatctaataaatagattttttgtggattcaaaagacatcacacctgatgactttattaattcctataatagtcaggcttcttctgatgaattagctgcttttaagggcatagctttaggtatttcattatatcctaacaattataatttaaagacagcctttgacagtgatgcataccatttgctagatactgcagtaagcattcataatcaagggaattatgactcagctttaaacatttataattatttacttcaattgccagtacctgaaaatattaaaattaatgctactgtatacaaaaatgttgcagcatcaaaaaatcctataataagtaaatatatctataaggaaagttctaaagatatagttcaaaatgccgtagaaatgtacagcaaacttaattttaattttgataatccaattacgtataaagctatgtcggattatttagatttctttacaagtgatcattttaataaatctgatatattaagttttgatgacaatggaattccaatagtaacctatagggaatttggtgctcaatacaattatgtgacaatatgccaatatgcactttatcttcatacaaaatatttacatggagatacatctgtattaagtcaatttataaaatgtaccgattttctcctcgatcacatggatagtgatggaagttttagatataaatttccttattactcctatgaatctcttggaactaattggacttcaagtatggctcaaggtgaagcattaaatgttttttctcgtgcttacgaaataactcacgatcataaatatattgatgccggtaacaaagcttttaactatttgataacaccaataagtcaaggtggcgttatggacactcttggaagcctcgaccctagatttaaaagtgatatattctttcaagagtatgtgaatacaacacctacttatacattaaatggatacatgtttacattactaggtatatatgactggaatcaactttcaaaaacagtatctgacataagtggtgatcaaacaagttattatttcaatgaaggcattaaaaccttgaaagtagtattaccttactatgacataggtggtttcaccagttatgatttaagccatctaatgacatctagggatgctaatcctgctttagattatcacaaggttcatatagatttactagaagcaatttactctataacaggtgataattactttatggatattagaaatcaatgggttaactacattaaacattaa

SEQ ID NO: 32 - DNA, 2214 bp, Clostridium autoethanogenum
atgttatctaaaaaagttataactactttaataatttctacagcattaatattttcaaaaactactgtaatcaatgctcagtctcaaagtccaactataacaagattatctggcccgaacagatactcaactaatatgaaaatagctgactatggctggagcggtcattctgattatgcaattattgtttcaggtgaaaactttccagatgcaatttgtgctgcacctttaagtgccaagtacaatgcacctttattaatgacagatggaaacactttaagtcctgaaatacaaaataaattgacaagcctaggcattaaaaatatatttttaattggtgggacaggcgctgtttcaagtgaaatagaaaatgaattaacccaaaaaaatttaaatgtaaaaagattgggtggatctacaagaattgaaacttccattgaaattgcaaaagaagttggaggtaatcagctatttgttgtttcatcagaaagttttgcagatgcattatccatttcatcctatgcttcaagtaaagctgaacctattgttttaactcctaaaagtgatattcctgatgtacttaaagactatttaagtactttaaaaccttcaaaaacctatgtgattggtggacaaggtgttattcctgacgataccgcttcttactttggtgattatgaaagactaagtggtcaaagcagatatgatacaaatgcaaatgtaattaaagaattttatgatctttcaaaagtacaaaaaatatacgtagcagcagaaataaactatccagatggactatctatttctcctctagctgccaaaaataattcacctgtaatactcgtaaatgacgatatgtcttccgaccagattagaatggttagaaacaataaagctacactaaaagagtgtgtggttgtaggtggagaagatgttactccagactatctaataaatagattttttgtggattcaaaagacatcacacctgatgactttattaattcctataatagtcaggcttcttctgatgaattagctgcttttaagggcatagctttaggtatttcattatatcctaacaattataatttaaagacagcctttgacagtgatgcataccatttgctagatactgcagtaagcattcataatcaagggaattatgactcagctttaaacatttataattatttacttcaattgccagtacctgaaaatattaaaattaatgctactgtatacaaaaatgttgcagcatcaaaaaatcctataataagtaaatatatctataaggaaagttctaaagatatagttcaaaatgccgtagaaatgtacagcaaacttaattttaattttgataatccaattacgtataaagctatgtcggattatttagatttctttacaagtgatcattttaataaatctgatatattaagttttgatgacaatggaattccaatagtaacctatagggaatttggtgctcaatacaattatgtgacaatatgccaatatgcactttatcttcatacaaaatatttacatggagatacatctgtattaagtcaatttataaaatgtaccgattttctcctcgatcacatggatagtgatggaagttttagatataaatttccttattactcctatgaatctcttggaactaattggacttcaagtatggctcaaggtgaagcattaaatgttttttctcgtgcttacgaaataactcacgatcataaatatattgatgccggtaacaaagcttttaactatttgataacaccaataagtcaaggtggcgttatggacactcttggaagcctcgaccctagatttaaaagtgatatattctttcaagagtatgtgaatacaacacctacttatacattaaatggatacatgtttacattactaggtatatatgactggaatcaactttcaaaaacagtatctgacataagtggtgatcaaacaagttattatttcaatgaaggcattaaaaccttgaaagtagtattaccttactatgacataggtggtttcaccagttatgatttaagccatctaatgacatctagggatgctaatcctgctttagattatcacaaggttcatatagatttactagaagcaatttactctataacaggtgataattactttatggatattagaaatcaatgggttaactacattaaacattaa

SEQ ID NO: 33 - protein, 737 aa, Clostridium autoethanogenum
MLSKKVITTLIISTALIFSKTTVINAQSQSPTITRLSGPNRYSTNMKIADYGWSGHSDYAIIVSGENFPDAICAAPLSAKYNAPLLMTDGNTLSPEIQNKLTSLGIKNIFLIGGTGAVSSEIENELTQKNLNVKRLGGSTRIETSIEIAKEVGGNQLFVVSSESFADALSISSYASSKAEPIVLTPKSDIPDVLKDYLSTLKPSKTYVIGGQGVIPDDTASYFGDYERLSGQSRYDTNANVIKEFYDLSKVQKIYVAAGINYPDGLSISPLAAKNNSPVILVNDDMSSDQIRMVRNNKATLKECVVVGGEDVTPDYLINRFFVDSKDITPDDFINSYNSQASSDELAAFKGIALGISLYPNNYNLKTAFDSDAYHLLDTAVSIHNQGNYDSALNIYNYLLQLPVPENIKINATVYKNVAASKNPIISKYIYKESSKDIVQNAVEMYSKLNFNFDNPITYKAMSDYLDFFTSDHFNKSDILSFDDNGIPIVTYREFGAQYNYVTICQYALYLHTKYLHGDTSVLSQFIKCTDFLLDHMDSDGSFRYKFPYYSYESLGTNWTSSMAQGEALNVFSRAYEITHDHKYIDAGNKAFNYLITPISQGGVMDTLGSLDPRFKSDIFFQEYVNTTPTYTLNGYMFTLLGIYDWNQLSKTVSDISGDQTSYYFNEGIKTLKVVLPYYDIGGFTSYDLSHLMTSRDANPALDYHKVHIDLLEAIYSITGDNYFMDIRNQWVNYIKH

SEQ ID NO: 34 - protein, 737 aa, Clostridium autoethanogenum
MLSKKVITTLIISTALIFSKTTVINAQSQSPTITRLSGPNRYSTNMKIADYGWSGHSDYAIIVSGENFPDAICAAPLSAKYNAPLLMTDGNTLSPEIQNKLTSLGIKNIFLIGGTGAVSSEIENELTQKNLNVKRLGGSTRIETSIEIAKEVGGNQLFVVSSESFADALSISSYASSKAEPIVLTPKSDIPDVLKDYLSTLKPSKTYVIGGQGVIPDDTASYFGDYERLSGQSRYDTNANVIKEFYDLSKVQKIYVAAEINYPDGLSISPLAAKNNSPVILVNDDMSSDQIRMVRNNKATLKECVVVGGEDVTPDYLINRFFVDSKDITPDDFINSYNSQASSDELAAFKGIALGISLYPNNYNLKTAFDSDAYHLLDTAVSIHNQGNYDSALNIYNYLLQLPVPENIKINATVYKNVAASKNPIISKYIYKESSKDIVQNAVEMYSKLNFNFDNPITYKAMSDYLDFFTSDHFNKSDILSFDDNGIPIVTYREFGAQYNYVTICQYALYLHTKYLHGDTSVLSQFIKCTDFLLDHMDSDGSFRYKFPYYSYESLGTNWTSSMAQGEALNVFSRAYEITHDHKYIDAGNKAFNYLITPISQGGVMDTLGSLDPRFKSDIFFQEYVNTTPTYTLNGYMFTLLGIYDWNQLSKTVSDISGDQTSYYFNEGIKTLKVVLPYYDIGGFTSYDLSHLMTSRDANPALDYHKVHIDLLEAIYSITGDNYFMDIRNQWVNYIKH

SEQ ID NO: 35 - DNA, 300 bp, Clostridium autoethanogenum
atgagtatgaaatggaaatacaagtgctggaacttggaatcaattcagtatcattttagtattcctaaaaaatcatataaaatatagtgatatatattaatttataacgcatagttacattgactgattatcaatgcaactatgcgttttttaataataacaaaatttatataggctgattatgacgatttattaaaaaagtattgactattttgataaatggatatatattataataaagatttacgattcgcgaatattttacaaaatatgaataaaaatgcagaggagagatgaaaa

SEQ ID NO: 36 - DNA, 300 bp, Clostridium autoethanogenum
atgagtatgaaatggaaatacaagtgctggaacttggaatcaattcagtatcattttagtattcctaaaaaatcatataaaatatagtgatatatattaatttataacgcatagttacattgactgattatcaatgcaactatgcgttttttaataataacaaaatttatataggatgattatgacgatttattaaaaaagtattgactattttgataaatggatatatattataataaagatttacgattcgcgaatattttacaaaatatgaataaaaatgcagaggagagatgaaaa

SEQ ID NO: 37 - DNA, 747 bp, Clostridium autoethanogenum
atgacaataaatgtagacttgtggaaaacattaattgatttaaaaaaatgcaaatgggttgatttaacacatgcctttggttcagatacaccaaagtggtctggctttaaggctgaaaaaattgaaactctttttacaattgagaaggatggtatatttgtcaatcaatatacttttccaggacagtatggaacacatatggatgccccaggacactttgcagcagggaaaagacttattgaggatatagaacttaaagaaatggttcttcctcttgtagttattgattgttcagaaaagtttaaagaaaatcctgattatgaacttacacttgaagatttgcttgcatttgaagctgagtacggtattattccagaaggttcttttgtggcaatgagaacggattggggtaaaaattggccagatcaagaaaagtgttcaaatgcagacagtgaaggaaatcttcattatccaggatgggcatatgaaacattggagtttttatatgaaacacgaaagatagcagcaagtggccatgaaccatttgatacagatgctccaataagacaagctgaatatggctttaaaggtgaaaattatatattaaatcaagataaatatcaaatcgaagtaatgacaaatttggataaagttccaccagtaggatcaataattttctgtgtggttccaaaagctaaaaatgctccaggatttccagtacgtgcttttgcaatagttaatgagtaa

SEQ ID NO: 38 - protein, 248 aa, Clostridium autoethanogenum
MTINVDLWKTLIDLKKCKWVDLTHAFGSDTPKWSGFKAEKIETLFTIEKDGIFVNQYTFPGQYGTHMDAPGHFAAGKRLIEDIELKEMVLPLVVIDCSEKFKENPDYELTLEDLLAFEAEYGIIPEGSFVAMRTDWGKNWPDQEKCSNADSEGNLHYPGWAYETLEFLYETRKIAASGHEPFDTDAPIRQAEYGFKGENYILNQDKYQIEVMTNLDKVPPVGSIIFCVVPKAKNAPGFPVRAFAIVNE

SEQ ID NO: 39 - DNA, 300 bp, Clostridium autoethanogenum
tcaaaaagcatcgtgcattacaggtgcatatactttgggaatattgatgctgcaacttttatggtttgagttttgggaaggacgtaagtctgaagaataatatttttaataacaaagtacaaagttcaaatatcagataaggataattttctttctgacgttggaaaatattaaaattataggtttcctgaggtacgagggaaatcatctttatctgttctttgctatttttactttgttatttaaaaaaatttgtatattagaaagttgagttcttaactaattcgaggggggatagct

SEQ ID NO: 40 - DNA, 300 bp, Clostridium autoethanogenum
tcaaaaagcatcgtgcattacaggtgcatatactttgggaatattgatgctgcaacttttatggtttgagttttgggaaggacgtaagtctgaagaataatatttttaataacaaagtacaaagttcaaatatcagacaaggataattttctttctgacgttggaaaatattaaaattataggtttcctgaggtacgagggaaatcatctttatctgttctttgctatttttactttgttatttaaaaaaatttgtatattagaaagttgagttcttaactaattcgaggggggatagct

SEQ ID NO: 41 - DNA, 660 bp, Clostridium autoethanogenum
atggccaatattcttgtaatagatgatgaagaggacattttagtacttgtaaaaaatgttcttgcgaaagatggacatgttgttacaattatcaataatccaaaaaaaattctgcttaatgaatacgcaaaatatgatcttattttattggatgttatgatgccggatatagatggttttgagttatgtgagaaaattcgtgacatagtggactgtccaattttgtttttaactgctaaaactatggaaaatgacgtaatgtttggacttggaataggtggtgatgattatataaaaaaaccttttagtacaggagaacttcgtgctagagtaaatgcccacctgaggagagaaaaaagagaaaagcacaatatgatttctatctctggtatgaaattcaatttggcaagcaaggaaattattatagaagataaaaaggtacctatgacaaaaagtgaatatgctatttgtgaatttcttgtacgcagcaagggacaggtgtttacaaaagagcaaatttacgaagctgtatacggatatgaaagggaaagtgacagctcagggattgcagagcatataaaaaatataagggcaaagcttgctatttttgatttatctccaattaaaacggtttgggggattggctatagatgggaataa

SEQ ID NO: 42 - protein, 219 aa, Clostridium autoethanogenum
MANILVIDDEEDILVLVKNVLAKDGHVVTIINNPKKILLNEYAKYDLILLDVMMPDIDGFELCEKIRDIVDCPILFLTAKTMENDVMFGLGIGGDDYIKKPFSTGELRARVNAHLRREKREKHNMISISGMKFNLASKEIIIEDKKVPMTKSEYAICEFLVRSKGQVFTKEQIYEAVYGYERESDSSGIAEHIKNIRAKLAIFDLSPIKTVWGIGYRWE

SEQ ID NO: 43 - DNA, 1308 bp, Clostridium autoethanogenum
atgttaaagtcaattttaacatcaaatcctttatttttatgtgataattttcttgacttaaggaaagaatacatctcaaaggaggagagaaataaaatgaggaaaaagcaaggagcttttaaaatagtaagcaatatattaattttattttttattacagtgttgtggttaccagttgttaatgcagaacctttaagtgaatcaaataaaaaagagatgaatagtacagaatcaggtttaaatataaatgctaggtcggcattgcttatggaacctgcaagtggaaaagtaatttttgagaagagttctcatgaaaaattagaacctgcttcagttacgaaaataatgactatgcttttaactatggaagcagtggactcaggaagaatatcattgtcagataaggtaaccgtaagtgaaaatgcaaaaaagatgggcggaagttcgatgttattagatactggagaagttagaagtgtggaagatttaataaagggaataggaatagcttcaggaaacgatgcagcagttgctatggcagaatatttaggaggaagtgaagatcaatttgtacagcttatgaataaaagagcagcagaacttggaatgaaagatacaagttttaaaaattgcacaggacttagtgaagatggacatttaactacagcctatgatatagcattaatgtctagagagttacttaagcatacaaggatactgaaatatacaggaacatatatggagacaatatcagaaggcagaaaaagtcctatagagttagtaaaccataataaattagtaagattttttaaagggtgtgatggattaaaaactgggtttaccagttctgctaagtactgtatatctgcaactgcaactagagatggtgcaagaatgttggctgtaataatgggctctcctacatataaggttaggaataaagatgcatctatgatgatgaattatggattttccaagtactttggaaagaatattttaaaaaagggagctgatgtagaaaagattcctctaaataaagcaggagatagattttttattgctaaagcttcagaagatttaaaggtggtagttgaaaagggaaaagaaagtaaaattacttataagtgtgtaatagataaaaataaaaagcaatacaaaaaaggcgaaaaagttgggtattgtgatgtatatgtaaatggagaggctttaggaaaagttaacttatatagcgacagggatgtaaaaaagttaggcatatttgggaactttaaagatagtttaaaaaatattttacagaaagggcaatag

SEQ ID NO: 44 - DNA, 1308 bp, Clostridium autoethanogenum
atgttaaagtcaattttaacatcaaatcctttatttttatgtgataattttcttgacttaaggaaagaatacatctcaaaggaggagagaaataaaatgaggaaaaagcaaggagcttttaaaatagtaagcaatatattaattttattttttattacagtgttgtggttaccagttgttaatgcagaacctttaagtgaatcaaataaaaaagagatgaatagtacagaatcaggtttaaatataaatgctaggtcggcattgcttatggaacctgcaagtggaaaagtaatttttgagaagagttctcatgaaaaattagaacctgcttcagttacgaaaataatgactatgcttttaactatggaagcagtggactcaggaagaatatcattgtcagataaggtaaccgtaagtgaaaatgcaaaaaagatgggcggaagttcgatgttattagatactggagaagttagaagtgtggaagatttaataaagggaataggaatagcttcaggaaacgatgcagcagttgctatggcagaatatttaggaggaagtgaagatcaatttgtacagcttatgaataaaagagcagcagaacttggaatgaaagatacaagttttaaaaattgcacaggacttagtgaagatggacatttaactacagcctatgatatagcattaatgtctagagagttacttaagcatacaaggatactgaaatatacaggaacatatatggagacaatatcagaaggcagaaaaagtcctatagagttagtaaaccataataaattagtaagattttttaaagggtgtgatggattaaaaactgggtttaccagttctgctaagtactgtatatctgcaactgcaactagagatggtgcaagaatgttggctgtaataatgggctctcctacatataaggtcaggaataaagatgcatctatgatgatgaattatggattttccaagtactttggaaagaatattttaaaaaagggagctgatgtagaaaagattcctctaaataaagcaggagatagattttttattgctaaagcttcagaagatttaaaggtggtagttgaaaagggaaaagaaagtaaaattacttataagtgtgtaatagataaaaataaaaagcaatacaaaaaaggcgaaaaagttgggtattgtgatgtatatgtaaatggagaggctttaggaaaagttaacttatatagcgacagggatgtaaaaaagttaggcatatttgggaactttaaagatagtttaaaaaatattttacagaaagggcaatag

SEQ ID NO: 45 - protein, 435 aa, Clostridium autoethanogenum
MLKSILTSNPLFLCDNFLDLRKEYISKEERNKMRKKQGAFKIVSNILILFFITVLWLPVVNAEPLSESNKKEMNSTESGLNINARSALLMEPASGKVIFEKSSHEKLEPASVTKIMTMLLTMEAVDSGRISLSDKVTVSENAKKMGGSSMLLDTGEVRSVEDLIKGIGIASGNDAAVAMAEYLGGSEDQFVQLMNKRAAELGMKDTSFKNCTGLSEDGHLTTAYDIALMSRELLKHTRILKYTGTYMETISEGRKSPIELVNHNKLVRFFKGCDGLKTGFTSSAKYCISATATRDGARMLAVIMGSPTYKVRNKDASMMMNYGFSKYFGKNILKKGADVEKIPLNKAGDRFFIAKASEDLKVVVEKGKESKITYKCVIDKNKKQYKKGEKVGYCDVYVNGEALGKVNLYSDRDVKKLGIFGNFKDSLKNILQKGQ

SEQ ID NO: 46 - DNA, 1212 bp, Clostridium autoethanogenum
atgaattcattaaaagatcttaaattcagtgacattattgatataaacactttaaaagatatccaagacaaactcgctaaaatagttattttttctgcagttacaacagatattcatggggttcctgtttgtaatgaaaacaatttcacgccattttgtaaacttattcgttcctccccaaaaggatgtaaaaactgtatatcctgtgattctcaagcaggctttatggctatgaaagataaaaagccaagagtatataaatgccacacaggactaattgattgtgctgcacctataatagtaaacgatatctatgtaggtgccgtgttgggcggacaggtattactaaaaggacaacaaacaagagattccatagacttaaattggatttctaaaaattatgagcttcctctagaaaaactaaaggaagctgctgaacgcatagaaattgttgaaagcgactacctacaaaattgtgtttcattttatacatttcttgcaaattatatagctgaaatgggccttcatactcttacacaggaaaaattattaaaagaaagtgaagaaaaaattaagcttgaacagtatgccaaaaaaatgcagttaaaaactatacaagctcaaattaatcctcactttctgtttaacactttaaatacaatagcaagtatggctctaattgaaaatgctcctacaacagaagaactaatatataatctctctgatttattaagatatagcttacgaaacttagaggaatttcccaaactcaaagatgaaattactaatataaaacgatatctttttatccaagctttaaggtacagcgatagaatttcctatgaaattaatatagatgaatctcttaatgaatatagaattccaaccatgattcttcagccacttgttgaaaactccctcgttcatggccttgaaaccaaaaaagaaggtggaaaaataactataaatagcacctccaattcaaaaaaagatataataattgaaataagtgataatggcagaggaattaatagcagcatacttaacctattaaataactcaaaagacctctctgatagtggtttaggtataggactgcaaaatactgacaataggctaaagcattattttggtagagattacggtctgaaaatagaaagtactcttgatgttggaacaaaagtatatatacgtattccaaaattgaaataa

SEQ ID NO: 47 - DNA, 1212 bp, Clostridium autoethanogenum
atgaattcattaaaagatcttaaattcagtgacattattgatataaacactttaaaagatatccaagacaaactcgctaaaatagttattttttctgcagttacaacagatattcatggggttcctgtttgtaatgaaaacaatttcacgccattttgtaaacttattcgttcctccccaaaaggatgtaaaaactgtatatcctgtgattctcaagcaggctttatggctatgaaagataaaaagccaagagtatataaatgccacacaggactaattgattgtgctgcacctataatagtaaacgatatctatgtaggtgccgtgttgggcggacaggtattactaaaaggacaacaaacaagagattccatagacttaaattggatttctaaaaattatgagcttcctctagaaaaactaaaggaagctgctgaacgcatagaaattgttgaaagcgactacctacaaaattgtgtttcattttatacatttcttgcaaattatatagctgaaatgggccttcatactcttacacaggaaaaattattaaaagaaagtgaagaaaaaattaagcttgaacagtatgccaaaaaaatgcagttaaaaactatacaagctcaaattaatcctcactttctgtttaacactttaaatacaatagcaagtatggctctaattgaaaatgctcctacaacagaagaactaatatataatctctctgatttattaagatatagcttacgaaacttagaggaatttcccaaactcaaagatgaaattactaatataaaacgatatctttttatccaagctttaaggtgcagcgatagaatttcctatgaaattaatatagatgaatctcttaatgaatatagaattccaaccatgattcttcagccacttgttgaaaactccctcgttcatggccttgaaaccaaaaaagaaggtggaaaaataactataaatagcacctccaattcaaaaaaagatataataattgaaataagtgataatggcagaggaattaatagcagcatacttaacctattaaataactcaaaagacctctctgatagtggtttaggtataggactgcaaaatactgacaataggctaaagcattattttggtagagattacggtctgaaaatagaaagtactcttgatgttggaacaaaagtatatatacgtattccaaaattgaaataa

SEQ ID NO: 48 - protein, 403 aa, Clostridium autoethanogenum
MNSLKDLKFSDIIDINTLKDIQDKLAKIVIFSAVTTDIHGVPVCNENNFTPFCKLIRSSPKGCKNCISCDSQAGFMAMKDKKPRVYKCHTGLIDCAAPIIVNDIYVGAVLGGQVLLKGQQTRDSIDLNWISKNYELPLEKLKEAAERIEIVESDYLQNCVSFYTFLANYIAEMGLHTLTQEKLLKESEEKIKLEQYAKKMQLKTIQAQINPHFLFNTLNTIASMALIENAPTTEELIYNLSDLLRYSLRNLEEFPKLKDEITNIKRYLFIQALRYSDRISYEINIDESLNEYRIPTMILQPLVENSLVHGLETKKEGGKITINSTSNSKKDIIIEISDNGRGINSSILNLLNNSKDLSDSGLGIGLQNTDNRLKHYFGRDYGLKIESTLDVGTKVYIRIPKLK

SEQ ID NO: 49 - protein, 403 aa, Clostridium autoethanogenum
MNSLKDLKFSDIIDINTLKDIQDKLAKIVIFSAVTTDIHGVPVCNENNFTPFCKLIRSSPKGCKNCISCDSQAGFMAMKDKKPRVYKCHTGLIDCAAPIIVNDIYVGAVLGGQVLLKGQQTRDSIDLNWISKNYELPLEKLKEAAERIEIVESDYLQNCVSFYTFLANYIAEMGLHTLTQEKLLKESEEKIKLEQYAKKMQLKTIQAQINPHFLFNTLNTIASMALIENAPTTEELIYNLSDLLRYSLRNLEEFPKLKDEITNIKRYLFIQALRCSDRISYEINIDESLNEYRIPTMILQPLVENSLVHGLETKKEGGKITINSTSNSKKDIIIEISDNGRGINSSILNLLNNSKDLSDSGLGIGLQNTDNRLKHYFGRDYGLKIESTLDVGTKVYIRIPKLK

SEQ ID NO: 50 - DNA, 300 bp, Clostridium autoethanogenum; identical to SEQ ID NO: 1

SEQ ID NO: 51 - DNA, 301 bp, Clostridium autoethanogenum; identical to SEQ ID NO: 2

SEQ ID NO: 52 - DNA, 297 bp, Clostridium autoethanogenum
atgaaatatgatgcattaggaatgattgaaacaaaaggtttagtaggttcaattgaagctgcagatgcaatggtaaaggcagctaatgtttatctaataggaaaagaacatgttggcggcggtcttgtaacagtaatggtaagaggagacgtaggagcagtaaaggcagctactgatgctggtgccgcagcagcacaacgtgttggagaattagtttctgttcatgttattcctcgtccacacgttgaagttgaaactatccttcctaaaactaatttaaaggaagatgaaaagtag

SEQ ID NO: 53 - protein, 98 aa, Clostridium autoethanogenum; identical to SEQ ID NO: 4

SEQ ID NO: 54 - DNA, 798 bp, Clostridium autoethanogenum
atggaagagaatagtgtaggctttaatatagaatatgaaagaaaaaagaaacttcttaaatcattaatagagcaactttccaaacttatagaggaaaaagattttcttttaaatgtgaaaaaggtaaatatagaaacaaaatatatgtgttccattggaagatatgaaatggagagaatgaacttaaattttgaaataagagctttgaaaaaagaaatatcccttagacaatcagcattaaataggggagaagttgtgtcagaagaatatatccaacaagttatgaaagaagaattgagagtttggcatgagaaggtaaatgctttttccaagcagataaaagatgcagaaatatttatgaaattaccaaagctttctgatgaagaaagcaaaagatttaaaagcttgtatagaaaacttataaagcttttgcatccggacatacataaatgtgatgaaagggacaaacttttatggcaaagggtttgtgaagcttataagaatggagacttagaagaactagagaacttaatgtatttagtggaagacaaaaatatgaatgatttattatataaacaagatgaaagtatagaagataaaattgaaaagttaaaagatttaatctttaaatgcctggataagatagataaaataaagaaaatgttcccattcacaattgaaaaggagatttcaaatgatcagtgggtaaaaggcaaaattgatgaaattcagataaatattcagttcttgaaaacctatagggaaagattaaaggttgtacttagcgaatttaaataa

SEQ ID NO: 55 - DNA, 798 bp, Clostridium autoethanogenum
atggaagagaatagtgtaggctttaatatagaatatgaaagaaaaaagaaacttcttaaatcattgatagagcaactttccaaacttatagaggaaaaagattttcttttaaatgtgaaaaaggtaaatatagaaacaaaatatatgtgttccattggaagatatgaaatggagagaatgaacttaaattttgaaataagagctttgaaaaaagaaatatcccttagacaatcagcattaaataggggagaagttgtgtcagaagaatatatccaacaagttatgaaagaagaattgagagtttggcatgagaaggtaaatgctttttccaagcagataaaagatgcagaaatatttatgaaattaccaaagctttctgatgaagaaagcaaaagatttaaaagcttgtatagaaaacttataaagcttttgcatccggacatacataaatgtgatgaaagggacaaacttttatggcaaagggtttgtgaagcttataagaatggagacttagaagaactagagaacttaatgtatttagtggaagacaaaaatatgaatgatttattatataaacaagatgaaagtatagaagataaaattgaaaagttaaaagatttaatctttaaatgcctggataagatagataaaataaagaaaatgttcccattcacaattgaaaaggagatttcaaatgatcagtgggtaaaaggcaaaattgatgaaattcagataaatattcagttcttgaaaacctatagggaaagattaaaggttgtacttagcgaatttaaataa

SEQ ID NO: 56 - protein, 265 aa, Clostridium autoethanogenum
MEENSVGFNIEYERKKKLLKSLIEQLSKLIEEKDFLLNVKKVNIETKYMCSIGRYEMERMNLNFEIRALKKEISLRQSALNRGEVVSEEYIQQVMKEELRVWHEKVNAFSKQIKDAEIFMKLPKLSDEESKRFKSLYRKLIKLLHPDIHKCDERDKLLWQRVCEAYKNGDLEELENLMYLVEDKNMNDLLYKQDESIEDKIEKLKDLIFKCLDKIDKIKKMFPFTIEKEISNDQWVKGKIDEIQINIQFLKTYRERLKVVLSEFK

SEQ ID NO: 57 - DNA, 5031 bp, Clostridium autoethanogenum
atgtttagaagatttaaaaaaataataacaaagatagctcttttggcaatgatatttagcatttttccacaaaatgttgtgaaggtacatgctgatccagcgccaattgatgaatcatcggctcctgtagttgcaactttatcatatccacaaggaatagctgtagatagcagcaataatatctatgtagctagtgatagtaataactatttttctaaaatatcaccagatggaacaatattaaaaacttatactgggtttgatggtccgtatggagtgtcaatggatagtagtgaaaacttgtatgtaactagtgatttaagtaatactgttactaaaatatctaaagatggaacaatattaaaaacttatactgggtttaacagtccatatggggtagcagttgacagtgatggaaatctctatgtagttaatcagaatggtaacactattcagaaagtgtcaaagagtggagaattattgaaaacttattctgatttaaatttaaacagtccatatggaatagcattggatagtagtaataatatctatgtagctgattatgggaatcatgcagtcaagaaggtatcatcagaagatggaacaatattgaaaacttatgatgtatcgggaccctgtggagtagcaatagatagtgacaacaatatttatatagctgaaaaagatgatggatgtgttgaaaagttatcacaagatggaatagtattagctgaatatactaattgtggtagttcactacgaggaatagccttagataaagataaaaacatctatgtgtctgatgagggtaacggttgtataaagaaaataatacaaccagcgccagcttcaacagcctgtgacataatcacatggacagcaccagcaagtccagcaatgaccgaaacaacagcaagcaagacagtaccaaatggagtaacaagcttaggggtagatgtagcagtaagtgcaaatgcaacctggaagttatatagtgatgcagtatgcagtactgaaatcacaaacaaaacaatgaatttaaatgtaggagtaaacacagcgtatattaaggtaacagcagaagatggaacaacaaaaaaaatttatacagtaaaaataacaagacaagccccggcggcaacagaaataacaagctttgataaaataaatgatgtattagcgggaaatgcaggaagtgcaacttattctgatgcagcagcggtaatagcagccctgccaacaacagtaaaggcaaataatagtgcagtgactgtaccagtaagcacatgggtggacacggaccactataatccaagtgcagcaggaagctatacctttacagcaacattaggagcaataccaacaggttatgcaaatagtgggaactatacagcaacagtaaaagtaattgtatcatcagctccaggaggtacagtaacagtagcaggagatagcaagataacaggcctaactataggaaacaagtacaaagtaacagtagatggagcagtaaaatatgtaaaggcagatggaacattatcagataatgaatcagatgcagcagcattaactggaacagaaataacaggcttgaccaatggaaagacatacaaagtagaagtctatgtacaaaatggaaacacaggatctagtggcttaagttggcagttagttggtaatgcagacatctcggaaggaataatgatgtatccaggtatagctatagctccagatggaacactttatattttatattcagataaaggaaatggcagtaaggcaacagtaatgaagtatgacaatataaataagatatggacagcagttggtggtggagttgtttcagcaggtgaggcagactgcacaactatagacatagataaagatggagtaccttatattggatatactgatatggcaaatggctttaaaggaacagtaatgaaatatgatagtaaaagtaatagctggacatcagtaggaaagccaggttttacgggatctcatttaggatatccaactattacaatagataaaattactggaacaccatatattgcatttaaaacaccatcagataaaggtggagcaacagtaatgaaatatgatagtacaagtgacagttggataactgtgggagatgaagacttcacagagaatgggataaatagtgcctctatagctatctataatggagtaccatattttgcatacgcagatgcgaataaaacatatgatgataaagcaacagtaatgaagtatgatggaaaaaactgggttacagtaggaaataaagatttttcaaaaggtaatgctggtatgctgatgatagctatagacggaaatggagtaccttatgtttcgtatgaagataccgcaaatgatggtaaggcaacggtaatgaaatatgacagtgaaactaaaaattgggtaactgttggaagtgaaggtttttcggcagaaagagcgaatagtttatcaatggctatagataaagatgggacaccatatattgtatatgaaaaaatagttggtacaaatattaaatcagtggtaatgaaatatgaaagtggaagttggaaaaccgttggagatccagatatttcactatatgtatccatggctatagacaagaatggagtaccttatattttatctggagataatgcaaatggttataaagcaacagtagaagcatatttaccaagtcaagcaggtactgtaacattagcagaaggttcagtaagtggagcagtagcaggagacagcaaagtaacaggcttaactataggaagcaaatataaggtaacagtagacggagcagtaaaatatgtaaaagcagatggaacattatcagataatgaatcagatgcagcagcattaactggtacagaaataataggcttgaccaatggaaagacatataaagtagaagcatacacacctccatctggaaactcgttttcaatacaggttacaggaaagccggtagaaacagtaaatagcttagatgtaacaccaaatatgacaggtggatatcactttaaacttccaaatgcaacaagggatgctctattaagtgatgttaattcagtaaatacagataaagttgatgtaactataagcaaaggaagcaaaagtataagtggagaatttccaaagactaatataaccaatgaaaatttgatagctggtgtagtacttccagttactgtatcaagtcctattattgtagctggaaacactattacttcaggaaactataatataagtataaaaatgattccgggaagtacagaaagtgattttgatatagcttttatatcaaagccaggttctcatagcggcggaagctctggtagttcatcaggaggtacttcagatcaaactactgttacaggtaatgtagtggaaggaaattcacaaagtcaagtttcaaatataacagctagtgtaactactgacagcagtggtaacaaaacagtttcaatgaaggcttcagaggtagcagcattaaaagcaccagacggaactgtaagtggtttaacagatgtttcaaaattaagtattaccacagataaaggaacaccagttacaattgggactgacggaacagttaaaattgaaagcttagcaaaaggaacaactaacaactttaatttaacttatgatttaggaaatggacaaaagattgttataggaacgatggaagttaaagttggaacagatggaaaagtaggcttacaaagtaacttaattgacccttatggaataataacagatgtatctacaggaaaacctatagatggagcaaatgtaacagtatattatgcaaacactgatagagataaagctgcagggaagaaaccagatacagaagtaccactccctggtatagatggatttaaacctaataataacaagaatccacaagtaagcgacagcaatggagcctatggatttatggtatttccaaattcagattactacgtagtagtaaataaagatgggtactataaatatgtaagtcctacaatttctgtagaaaaggaaatcgtaaaatgggatataagaatgaataaagaagatgcttcaagtaaagtaacaggagtacagcgtttgtccggacaaaataggatagatacagctttagatattgcaaaggcaacttatccagggaaaatttcaaatgtagtaattgcttcagcacaaaattacccagatgctttgtcaggaagtgtactagcatacaaactaaatgcaccaatgttgcttgtaggaagtacagatgaagatcagcaaaaagtactagcttatattaaatcctgcatggataatacaggttcggtttatatacttggaggaaacggagcagtaagcaaagaatttgaagataaagttacagctaatggttttaagaatattacaagactaggtggagcagacagatatgaaacttcagaaaagatagcagaaaagttaaatgtaaataaagaaactcctgtagttatagtttcgggagaggattatgcagatgcagcttcaatatctaatatagcagcagcaaaccaatatcctattttattagttagaaaaaatgaaatttcagatacaataaagaaagaaatttcaactataaatccaactaaagtatttattataggattgcagggatcaatagacacagaagttgaaaatactattgcacagattataccaatagagaagtcaaatattacgagaataggtggatcagaccgttatgaaacttctcttgaagtaggaaaatattttaagttagatggaaatgaagcatgtatagcatctggtaataattttccggatgcacttgctggaagcatatatgctggaaagcataatgccccaattgttttagttaatacaaaattgtcagataatcaaacttcatatttgaaagacagaaaattatctggagcaaccatatttggtggagaaggtgtagtagataagaatattcaacaacaactctctcaaataattggtaagtaa

SEQ ID NO: 58 - DNA, 5030 bp, Clostridium autoethanogenum
atgtttagaagatttaaaaaaataataacaaagatagctcttttggcaatgatatttagcatttttccacaaaatgttgtgaaggtacatgctgatccagcgccaattgatgaatcatcggctcctgtagttgcaactttatcatatccacaaggaatagctgtagatagcagcaataatatctatgtagctagtgatagtaataactatttttctaaaatatcaccagatggaacaatattaaaaacttatactgggtttgatggtccgtatggagtgtcaatggatagtagtgaaaacttgtatgtaactagtgatttaagtaatactgttactaaaatatctaaagatggaacaatattaaaaacttatactgggtttaacagtccatatggggtagcagttgacagtgatggaaatctctatgtagttaatcagaatggtaacactattcagaaagtgtcaaagagtggagaattattgaaaacttattctgatttaaatttaaacagtccatatggaatagcattggatagtagtaataatatctatgtagctgattatgggaatcatgcagtcaagaaggtatcatcagaagatggaacaatattgaaaacttatgatgtatcgggaccctgtggagtagcaatagatagtgacaacaatatttatatagctgaaaaagatgatggatgtgttgaaaagttatcacaagatggaatagtattagctgaatatactaattgtggtagttcactacgaggaatagccttagataaagataaaaacatctatgtgtctgatgagggtaacggttgtataaagaaaataatacaaccagcgccagcttcaacagcctgtgacataatcacatggacagcaccagcaagtccagcaatgaccgaaacaacagcaagcaagacagtaccaaatggagtaacaagcttaggggtagatgtagcagtaagtgcaaatgcaacctggaagttatatagtgatgcagtatgcagtactgaaatcacaaacaaaacaatgaatttaaatgtaggagtaaacacagcgtatattaaggtaacagcagaagatggaacaacaaaaaaatttatacagtaaaaataacaagacaagccccggcggcaacagaaataacaagctttgataaaataaatgatgtattagcgggaaatgcaggaagtgcaacttattctgatgcagcagcggtaatagcagccctgccaacaacagtaaaggcaaataatagtgcagtgactgtaccagtaagcacatgggtggacacggaccactataatccaagtgcagcaggaagctatacctttacagcaacattaggagcaataccaacaggttatgcaaatagtgggaactatacagcaacagtaaaagtaattgtatcatcagctccaggaggtacagtaacagtagcaggagatagcaagataacaggcctaactataggaaacaagtacaaagtaacagtagatggagcagtaaaatatgtaaaggcagatggaacattatcagataatgaatcagatgcagcagcattaactggaacagaaataacaggcttgaccaatggaaagacatacaaagtagaagtctatgtacaaaatggaaacacaggatctagtggcttaagttggcagttagttggtaatgcagacatctcggaaggaataatgatgtatccaggtatagctatagctccagatggaacactttatattttatattcagataaaggaaatggcagtaaggcaacagtaatgaagtatgacaatataaataagatatggacagcagttggtggtggagttgtttcagcaggtgaggcagactgcacaactatagacatagataaagatggagtaccttatattggatatactgatatggcaaatggctttaaaggaacagtaatgaaatatgatagtaaaagtaatagctggacatcagtaggaaagccaggttttacgggatctcatttaggatatccaactattacaatagataaaattactggaacaccatatattgcatttaaaacaccatcagataaaggtggagcaacagtaatgaaatatgatagtacaagtgacagttggataactgtgggagatgaagacttcacagagaatgggataaatagtgcctctatagctatctataatggagtaccatattttgcatacgcagatgcgaataaaacatatgatgataaagcaacagtaatgaagtatgatggaaaaaactgggttacagtaggaaataaagatttttcaaaaggtaatgctggtatgctgatgatagctatagacggaaatggagtaccttatgtttcgtatgaagataccgcaaatgatggtaaggcaacggtaatgaaatatgacagtgaaactaaaaattgggtaactgttggaagtgaaggtttttcggcagaaagagcgaatagtttatcaatggctatagataaagatgggacaccatatattgtatatgaaaaaatagttggtacaaatattaaatcagtggtaatgaaatatgaaagtggaagttggaaaaccgttggagatccagatatttcactatatgtatccatggctatagacaagaatggagtaccttatattttatctggagataatgcaaatggttataaagcaacagtagaagcatatttaccaagtcaagcaggtactgtaacattagcagaaggttcagtaagtggagcagtagcaggagacagcaaagtaacaggcttaactataggaagcaaatataaggtaacagtagacggagcagtaaaatatgtaaaagcagatggaacattatcagataatgaatcagatgcagcagcattaactggtacagaaataataggcttgaccaatggaaagacatataaagtagaagcatacacacctccatctggaaactcgttttcaatacaggttacaggaaagccggtagaaacagtaaatagcttagatgtaacaccaaatatgacaggtggatatcactttaaacttccaaatgcaacaagggatgctctattaagtgatgttaattcagtaaatacagataaagttgatgtaactataagcaaaggaagcaaaagtataagtggagaatttccaaagactaatataaccaatgaaaatttgatagctggtgtagtacttccagttactgtatcaagtcctattattgtagctggaaacactattacttcaggaaactataatataagtataaaaatgattccgggaagtacagaaagtgattttgatatagcttttatatcaaagccaggttctcatagcggcggaagctctggtagttcatcaggaggtacttcagatcaaactactgttacaggtaatgtagtggaaggaaattcacaaagtcaagtttcaaatataacagctagtgtaactactgacagcagtggtaacaaaacagtttcaatgaaggcttcagaggtagcagcattaaaagcaccagacggaactgtaagtggtttaacagatgtttcaaaattaagtattaccacagataaaggaacaccagttacaattgggactgacggaacagttaaaattgaaagcttagcaaaaggaacaactaacaactttaatttaacttatgatttaggaaatggacaaaagattgttataggaacgatggaagttaaagttggaacagatggaaaagtaggcttacaaagtaacttaattgacccttatggaataataacagatgtatctacaggaaaacctatagatggagcaaatgtaacagtatattatgcaaacactgatagagataaagctgcagggaagaaaccagatacagaagtaccactccctggtatagatggatttaaacctaataataacaagaatccacaagtaagcgacagcaatggagcctatggatttatggtatttccaaattcagattactacgtagtagtaaataaagatgggtactataaatatgtaagtcctacaatttctgtagaaaaggaaatcgtaaaatgggatataagaatgaataaagaagatgcttcaagtaaagtaacaggagtacagcgtttgtccggacaaaataggatagatacagctttagatattgcaaaggcaacttatccagggaaaatttcaaatgtagtaattgcttcagcacaaaattacccagatgctttgtcaggaagtgtactagcatacaaactaaatgcaccaatgttgcttgtaggaagtacagatgaagatcagcaaaaagtactagcttatattaaatcctgcatggataatacaggttcggtttatatacttggaggaaacggagcagtaagcaaagaatttgaagataaagttacagctaatggttttaagaatattacaagactaggtggagcagacagatatgaaacttcagaaaagatagcagaaaagttaaatgtaaataaagaaactcctgtagttatagtttcgggagaggattatgcagatgcagcttcaatatctaatatagcagcagcaaaccaatatcctattttattagttagaaaaaatgaaatttcagatacaataaagaaagaaatttcaactataaatccaactaaagtatttattataggattgcagggatcaatagacacagaagttgaaaatactattgcacagattataccaatagagaagtcaaatattacgagaataggtggatcagaccgttatgaaacttctcttgaagtaggaaaatattttaagttagatggaaatgaagcatgtatagcatctggtaataattttccggatgcacttgctggaagcatatatgctggaaagcataatgccccaattgttttagttaatacaaaattgtcagataatcaaacttcatatttgaaagacagaaaattatctggagcaaccatatttggtggagaaggtgtagtagataagaatattcaacaacaactctctcaaataattggtaagtaa

SEQ ID NO: 59 - protein, 1676 aa, Clostridium autoethanogenum
MFRRFKKIITKIALLAMIFSIFPQNVVKVHADPAPIDESSAPVVATLSYPQGIAVDSSNNIYVASDSNNYFSKISPDGTILKTYTGFDGPYGVSMDSSENLYVTSDLSNTVTKISKDGTILKTYTGFNSPYGVAVDSDGNLYVVNQNGNTIQKVSKSGELLKTYSDLNLNSPYGIALDSSNNIYVADYGNHAVKKVSSEDGTILKTYDVSGPCGVAIDSDNNIYIAEKDDGCVEKLSQDGIVLAEYTNCGSSLRGIALDKDKNIYVSDEGNGCIKKIIQPAPASTACDIITWTAPASPAMTETTASKTVPNGVTSLGVDVAVSANATWKLYSDAVCSTEITNKTMNLNVGVNTAYIKVTAEDGTTKKIYTVKITRQAPAATEITSFDKINDVLAGNAGSATYSDAAAVIAALPTTVKANNSAVTVPVSTWVDTDHYNPSAAGSYTFTATLGAIPTGYANSGNYTATVKVIVSSAPGGTVTVAGDSKITGLTIGNKYKVTVDGAVKYVKADGTLSDNESDAAALTGTEITGLTNGKTYKVEVYVQNGNTGSSGLSWQLVGNADISEGIMMYPGIAIAPDGTLYILYSDKGNGSKATVMKYDNINKIWTAVGGGVVSAGEADCTTIDIDKDGVPYIGYTDMANGFKGTVMKYDSKSNSWTSVGKPGFTGSHLGYPTITIDKITGTPYIAFKTPSDKGGATVMKYDSTSDSWITVGDEDFTENGINSASIAIYNGVPYFAYADANKTYDDKATVMKYDGKNWVTVGNKDFSKGNAGMLMIAIDGNGVPYVSYEDTANDGKATVMKYDSETKNWVTVGSEGFSAERANSLSMAIDKDGTPYIVYEKIVGTNIKSVVMKYESGSWKTVGDPDISLYVSMAIDKNGVPYILSGDNANGYKATVEAYLPSQAGTVTLAEGSVSGAVAGDSKVTGLTIGSKYKVTVDGAVKYVKADGTLSDNESDAAALTGTEIIGLTNGKTYKVEAYTPPSGNSFSIQVTGKPVETVNSLDVTPNMTGGYHFKLPNATRDALLSDVNSVNTDKVDVTISKGSKSISGEFPKTNITNENLIAGVVLPVTVSSPIIVAGNTITSGNYNISIKMIPGSTESDFDIAFISKPGSHSGGSSGSSSGGTSDQTTVTGNVVEGNSQSQVSNITASVTTDSSGNKTVSMKASEVAALKAPDGTVSGLTDVSKLSITTDKGTPVTIGTDGTVKIESLAKGTTNNFNLTYDLGNGQKIVIGTMEVKVGTDGKVGLQSNLIDPYGIITDVSTGKPIDGANVTVYYANTDRDKAAGKKPDTEVPLPGIDGFKPNNNKNPQVSDSNGAYGFMVFPNSDYYVVVNKDGYYKYVSPTISVEKEIVKWDIRMNKEDASSKVTGVQRLSGQNRIDTALDIAKATYPGKISNVVIASAQNYPDALSGSVLAYKLNAPMLLVGSTDEDQQKVLAYIKSCMDNTGSVYILGGNGAVSKEFEDKVTANGFKNITRLGGADRYETSEKIAEKLNVNKETPVVIVSGEDYADAASISNIAAANQYPILLVRKNEISDTIKKEISTINPTKVFIIGLQGSIDTEVENTIAQIIPIEKSNITRIGGSDRYETSLEVGKYFKLDGNEACIASGNNFPDALAGSIYAGKHNAPIVLVNTKLSDNQTSYLKDRKLSGATIFGGEGVVDKNIQQQLSQIIGK

SEQ ID NO: 60 - protein, 370 aa, Clostridium autoethanogenum
MFRRFKKIITKIALLAMIFSIFPQNVVKVHADPAPIDESSAPVVATLSYPQGIAVDSSNNIYVASDSNNYFSKISPDGTILKTYTGFDGPYGVSMDSSENLYVTSDLSNTVTKISKDGTILKTYTGFNSPYGVAVDSDGNLYVVNQNGNTIQKVSKSGELLKTYSDLNLNSPYGIALDSSNNIYVADYGNHAVKKVSSEDGTILKTYDVSGPCGVAIDSDNNIYIAEKDDGCVEKLSQDGIVLAEYTNCGSSLRGIALDKDKNIYVSDEGNGCIKKIIQPAPASTACDIITWTAPASPAMTETTASKTVPNGVTSLGVDVAVSANATWKLYSDAVCSTEITNKTMNLNVGVNTAYIKVTAEDGTTKKFIQ

SEQ ID NO: 61 - DNA, 1092 bp, Clostridium autoethanogenum
gtgaaatgtaaagcgcttagtaaaaaaagtataacagctattattgctatggtatttgttgcttctacatttattggatgtgatggtaaaagcagtagtgaatcgactaaaaaaaatacaattaaaatagttgacatgactcatagaaatgttaaggtacctgccgatattaagagtgttttatcaatcagtcctccagctacaatgctaacatatatgcttgcgccagacaaacttatgggatggaatttgaaaatgacaggtaagtacatacctgataaatacaagaaattacctgttgtgggaggatggtatggtacaaccaaaggtaattatgaaaaatatatgtcgctgaaacctgatttgataattgatgaaggacataatgctgtccaagctgatgcagtaaaaaaggacacagaagaagaccagaagaacctgggaaatatcccggttgtaggtgtaacagatactgttactctggcaaattttccgaattatataagatttgtgggtaagatgctcaatgagagccagcaggccgaaaaactagtggccttctataataaagtaataaatcaagctaaaagcgtagcagctactattccagataatcaaaaagtaaaggtatattatgcagaaggctcaaatggtcttcagactgccccggctaaatctatacacacacagcctatagatatttgtggtggaatcaatgtagcacaggtagcattagataaaatgggtatggttgatgtatctccagaacaggtactaaactggaacccagatgtgattatagctactgatgctaatttttataaaggaatttattccaatcctgtatggaaagggattaaagctgttaaaaatcataaggtatatcttgtgccaagcgatccttttaattggtacgacaggtctcctagtgtaaacataatccttggggtgccatggactgctaaaatactgtatccagataaatttcagaatatggatttaaatagccttatcaaggaattttatactgatttttatcattataatcttacagacagtgatgttaataaattattaaacaccaaacaataa

SEQ ID NO: 62 - DNA, 1091 bp, Clostridium autoethanogenum
gtgaaatgtaaagcgcttagtaaaaaagtataacagctattattgctatggtatttgttgcttctacatttattggatgtgatggtaaaagcagtagtgaatcgactaaaaaaaatacaattaaaatagttgacatgactcatagaaatgttaaggtacctgccgatattaagagtgttttatcaatcagtcctccagctacaatgctaacatatatgcttgcgccagacaaacttatgggatggaatttgaaaatgacaggtaagtacatacctgataaatacaagaaattacctgttgtgggaggatggtatggtacaaccaaaggtaattatgaaaaatatatgtcgctgaaacctgatttgataattgatgaaggacataatgctgtccaagctgatgcagtaaaaaaggacacagaagaagaccagaagaacctgggaaatatcccggttgtaggtgtaacagatactgttactctggcaaattttccgaattatataagatttgtgggtaagatgctcaatgagagccagcaggccgaaaaactagtggccttctataataaagtaataaatcaagctaaaagcgtagcagctactattccagataatcaaaaagtaaaggtatattatgcagaaggctcaaatggtcttcagactgccccggctaaatctatacacacacagcctatagatatttgtggtggaatcaatgtagcacaggtagcattagataaaatgggtatggttgatgtatctccagaacaggtactaaactggaacccagatgtgattatagctactgatgctaatttttataaaggaatttattccaatcctgtatggaaagggattaaagctgttaaaaatcataaggtatatcttgtgccaagcgatccttttaattggtacgacaggtctcctagtgtaaacataatccttggggtgccatggactgctaaaatactgtatccagataaatttcagaatatggatttaaatagccttatcaaggaattttatactgatttttatcattataatcttacagacagtgatgttaataaattattaaacaccaaacaataa

SEQ ID NO: 63 - protein, 363 aa, Clostridium autoethanogenum
VKCKALSKKSITAIIAMVFVASTFIGCDGKSSSESTKKNTIKIVDMTHRNVKVPADIKSVLSISPPATMLTYMLAPDKLMGWNLKMTGKYIPDKYKKLPVVGGWYGTTKGNYEKYMSLKPDLIIDEGHNAVQADAVKKDTEEDQKNLGNIPVVGVTDTVTLANFPNYIRFVGKMLNESQQAEKLVAFYNKVINQAKSVAATIPDNQKVKVYYAEGSNGLQTAPAKSIHTQPIDICGGINVAQVALDKMGMVDVSPEQVLNWNPDVIIATDANFYKGIYSNPVWKGIKAVKNHKVYLVPSDPFNWYDRSPSVNIILGVPWTAKILYPDKFQNMDLNSLIKEFYTDFYHYNLTDSDVNKLLNTKQ

SEQ ID NO: 64 - protein, 363 aa, Clostridium autoethanogenum; identical to SEQ ID NO: 63

SEQ ID NO: 65 - DNA, 726 bp, Clostridium autoethanogenum
atgtgggtattatttgcatttggttcagcattttttgctggaattacagctattttagctaagatcggaataaaaaaaacagattctaatctggcaactgcaatcaggactattgctatattaattttttcatggctcatagttcttattgttggttccaaaaatacgatttatcaaataagtggacaaagtttactttttttgatattgtcaggactggcaacaggagcttcatggctttgttattttaaggctcttcagcttgggaatgtaaataaagtaacaccaatagataaatcaagtacagtattaactatgattttagcttttatcttattaggggaaaagattacttggataaaattcattggtatgtgtgctataggaataggcacatatatgatgataactaagaaggaagtgaaaaacaaagaaattgtagatagcagatggttattttatgctgcgctgtcagcggtgtttgcaagtttgacttcaattcttggaaaagtgggaatcagtggagttgaatcaaatttaggaactgcaatcagaaccattgttgtattaattatggcgtgggcagtagtttttgtttcgaaaaacaaaatgaaattaaaaatattgataaaaggagctgcttgtttatttgtctttcaggaattacaacaggagcttcgtggctttgctattacagagcgctgcaaaaaggtcttgcaagcgtag

SEQ ID NO: 66 - DNA, 727 bp, Clostridium autoethanogenum
atgtgggtattatttgcatttggttcagcattttttgctggaattacagctattttagctaagatcggaataaaaaaaacagattctaatctggcaactgcaatcaggactattgctatattaattttttcatggctcatagttcttattgttggttccaaaaatacgatttatcaaataagtggacaaagtttactttttttgatattgtcaggactggcaacaggagcttcatggctttgttattttaaggctcttcagcttgggaatgtaaataaagtaacaccaatagataaatcaagtacagtattaactatgattttagcttttatcttattaggggaaaagattacttggataaaattcattggtatgtgtgctataggaataggcacatatatgatgataactaagaaggaagtgaaaaacaaagaaattgtagatagcagatggttattttatgctgcgctgtcagcggtgtttgcaagtttgacttcaattcttggaaaagtgggaatcagtggagttgaatcaaatttaggaactgcaatcagaaccattgttgtattaattatggcgtgggcagtagtttttgtttcgaaaaaacaaaatgaaattaaaaatattgataaaaggagctgcttgtttatttgtctttcaggaattacaacaggagcttcgtggctttgctattacagagcgctgcaaaaaggtcttgcaagcgtag

SEQ ID NO: 67 - protein, 241 aa, Clostridium autoethanogenum
MWVLFAFGSAFFAGITAILAKIGIKKTDSNLATAIRTIAILIFSWLIVLIVGSKNTIYQISGQSLLFLILSGLATGASWLCYFKALQLGNVNKVTPIDKSSTVLTMILAFILLGEKITWIKFIGMCAIGIGTYMMITKKEVKNKEIVDSRWLFYAALSAVFASLTSILGKVGISGVESNLGTAIRTIVVLIMAWAVVFVSKNKMKLKILIKGAACLFVFQELQQELRGFAITERCKKVLQA

SEQ ID NO: 68 - protein, 242 aa, Clostridium autoethanogenum
MWVLFAFGSAFFAGITAILAKIGIKKTDSNLATAIRTIAILIFSWLIVLIVGSKNTIYQISGQSLLFLILSGLATGASWLCYFKALQLGNVNKVTPIDKSSTVLTMILAFILLGEKITWIKFIGMCAIGIGTYMMITKKEVKNKEIVDSRWLFYAALSAVFASLTSILGKVGISGVESNLGTAIRTIVVLIMAWAVVFVSKKQNEIKNIDKRSCLFICLSGITTGASWLCYYRALQKGLASV

SEQ ID NO: 69 - DNA, 750 bp, Clostridium autoethanogenum
ttgctaacagaacagcgccataagataatattagagaaattaaagcaaaatggaattgtaaaggttaacgatttagtaaaccttttaaatacatctgagtctactataagacgtgatttaacttatcttgaaaatataaatgtactaaagagaattcatggtggtgctacaattccaaaaggccgtctcatagaaccaacttataatgaaaaacaaattcaaaatgtagatcaaaaaagaaaaatagcaaaattcgcagcatcctatatagaagaaggagattgtatatatttagatgcaggaacttctacttttgaaatgattcaatatataaataaaaaatctctgatagtagtaactaatggattaaatcatataaatgctattattgaaaacaatataaatggatatatactaggaggaaaagttaaaaatagtacaaaagctgttataggatgcgatgcacttaaaagtattgaaaaatttagatttgacaaatgctttttaggtataaacggcattcatctaaaatacggatttactactcccgactctgaagaagctatacttaaagaaaatgccattaaacattcaggtcaaagctacatccttgcagatgagagtaaatttggagaagtaagttttgtaaaggtaggaaatttagatcaagcttccataattacaaactgtaaaatagaaaattatgaaaaatatatacaaaaaactaaagtaaaggttgtgacagattaa

SEQ ID NO: 70 - DNA, 750 bp, Clostridium autoethanogenum
ttgctaacagaacagcgccataagataatattagagaaattaaagcaaaatggaattgtaaaggttaacgatttagtaaaccttttaaatacatctgagtctactataagacgtgatttaacttatcttgaaaatataaatgcactaaagagaattcatggtggtgctacaattccaaaaggccgtctcatagaaccaacttataatgaaaaacaaattcaaaatgtagatcaaaaaagaaaaatagcaaaattcgcagcatcctatatagaagaaggagattgtatatatttagatgcaggaacttctacttttgaaatgattcaatatataaataaaaaatctctgatagtagtaactaatggattaaatcatataaatgctattattgaaaacaatataaatggatatatactaggaggaaaagttaaaaatagtacaaaagctgttataggatgcgatgcacttaaaagtattgaaaaatttagatttgacaaatgctttttaggtataaacggcattcatctaaaatacggatttactactcccgactctgaagaagctatacttaaagaaaatgccattaaacattcaggtcaaagctacatccttgcagatgagagtaaatttggagaagtaagttttgtaaaggtaggaaatttagatcaagcttccataattacaaactgtaaaatagaaaattatgaaaaatatatacaaaaaactaaagtaaaggttgtgacagattaa

SEQ ID NO: 71 - protein, 249 aa, Clostridium autoethanogenum
MLTEQRHKIILEKLKQNGIVKVNDLVNLLNTSESTIRRDLTYLENINVLKRIHGGATIPKGRLIEPTYNEKQIQNVDQKRKIAKFAASYIEEGDCIYLDAGTSTFEMIQYINKKSLIVVTNGLNHINAIIENNINGYILGGKVKNSTKAVIGCDALKSIEKFRFDKCFLGINGIHLKYGFTTPDSEEAILKENAIKHSGQSYILADESKFGEVSFVKVGNLDQASIITNCKIENYEKYIQKTKVKVVTD

SEQ ID NO: 72 - protein, 249 aa, Clostridium autoethanogenum
MLTEQRHKIILEKLKQNGIVKVNDLVNLLNTSESTIRRDLTYLENINALKRIHGGATIPKGRLIEPTYNEKQIQNVDQKRKIAKFAASYIEEGDCIYLDAGTSTFEMIQYINKKSLIVVTNGLNHINAIIENNINGYILGGKVKNSTKAVIGCDALKSIEKFRFDKCFLGINGIHLKYGFTTPDSEEAILKENAIKHSGQSYILADESKFGEVSFVKVGNLDQASIITNCKIENYEKYIQKTKVKVVTD

SEQ ID NO: 73 - DNA, 537 bp, Clostridium autoethanogenum
atggtaaggcaagcttctgtatttgcaggaggtcttgtttgtgtagctagaaattctgtaacagcaggttatgtaaaaaagaatggagcattagacggagtatctcatgctattgaaactggtagggtattttataaaggtttaaagcaaaatgtagaaagtgctccagagagtgctgcagaatttttaaaaggagaagtagttatagaaggaaaagtggatgagattatcttgaatactacaggtggatttgacgttggggtagttaaagttaaagattatgaaattacattttggaatgaatacatgactcttgagaaaaatggtgagagacttgcaacttttccggatcttataatgacttttgatagtataactggaatgcctgtaactagtgctgaaattaaacaaaatcaggttgttaaaattatgaagacaagtaagaaaaatttaaagcttggaagtggaatgaaggataaaagtctacttgaacaagcaggaaaaattataaataaagatattttaaattacatatag

SEQ ID NO: 74 - DNA, 487 bp, Clostridium autoethanogenum
gtggctaggaagataaaaattgatgaagaaattttaaattatgctgtttttggaggatgcatactaggaggcggcggaggaggttctagaaaattaggaatggagtcaggaaaggcagctttaaaatatggcaatctcgaattgattgatataaatgacattcctgaagatacaattataattacagcttcagcagttggagctcctgctgctagtcttcaatatgtactgccagaatatcatataagaactattaaattatttgaggaaaatacaggtataaaaataggcggaataataacaaatgaaaatggcggcgcttctaccatgaatgggtggacagaagctgttgctttagatataccttttattgatgcaccttgtaatggaagagctcatcctacaggtgttatgggaagtatgaatttaaataatgtggaaggatatgtttcctgtcaggctgcagtgggggggaatcctgaaataa

SEQ ID NO: 75 - protein, 161 aa, Clostridium autoethanogenum
VARKIKIDEEILNYAVFGGCILGGGGGGSRKLGMESGKAALKYGNLELIDINDIPEDTIIITASAVGAPAASLQYVLPEYHIRTIKLFEENTGIKIGGIITNENGGASTMNGWTEAVALDIPFIDAPCNGRAHPTGVMGSMNLNNVEGYVSCQAAVGGILK

SEQ ID NO: 76 - protein, 357 aa, Clostridium autoethanogenum
VARKIKIDEEILNYAVFGGCILGGGGGGSRKLGMESGKAALKYGNLELIDINDIPEDTIIITASAVGAPAASLQYVLPEYHIRTIKLFEENTGIKIGGIITNENGGASTMNGWTEAVALDIPFIDAPCNGRAHPTGVMGSMNLNNVEGYVSCQAAVGGNPEINKYLETFIRGSIGNASGMVRQASVFAGGLVCVARNSVTAGYVKKNGALDGVSHAIETGRVFYKGLKQNVESAPESAAEFLKGEVVIEGKVDEIILNTTGGFDVGVVKVKDYEITFWNEYMTLEKNGERLATFPDLIMTFDSITGMPVTSAEIKQNQVVKIMKTSKKNLKLGSGMKDKSLLEQAGKIINKDILNYI

SEQ ID NO: 77 - DNA, 1767 bp, Clostridium autoethanogenum
atgaatccatatgctgatgaacttattttttcagatgtcttatttacatttaagaaaggtgatcatatgaaagtagatataatcataaaagatgtttatatttttaatgcttattttaaaaagtttgtgtatggagatgtgtcaatattaaatggaaggtttttatatatagggaaggattgctataaaaaaataagtgcaagaaagattatagaaggtaacaataaatatctaatacctggccttattgatatacatatgcatattgaaagttcaatgactacaccaacaaactttgcaaatgctgtcattaaaaatggagttacaacagttgttgctgatccacatgaaattgcaaatgtatttggaatagaaggcataaaagctatgataaattctgataggcagcatataatggatatattttatggcattccaagttctgtaccatcaacatcatctaaacttgaaactacaggtggacaaatagggataaaggaagtatcagaacttataaaaaatgaccgtatatggtgcattggtgaagttatgaattttaaggatttagtttatagtgaagattcacttataaataatataattaaattgataaaaaaagaaaaacctcatttcaaaatagaaggacactgtccaaaaatatcaggatttgatctagcaagatacatatatcaaggggtagatgcagatcatactcagcaaactccaaaatctattgaggaaaagatattaaatggcatgtttttagaaattcaggaaaaatcaatgacaaaagaaaacatggaatatttaatttccaatgatttatatgagcatttttgctttgttactgatgatacaatggcagataaattaattacttcgcatttgaatggacttgttaaaaaagctattgacatgggtatgaagccggaaatggctgtttatgcaagtacttttattccagcaagaagaatgaatttttacgatagaggaagcattgcacctggaaagatagctgattttattttattggatgatttaaaatcatttagtataaatgaagtatataaaaatgggaaactagcatgtaaggatattggagggaaaaaagaacatgaatttccagagaacttttataaaagcataaaaatgaatccagttagtgattcagacttttcagttaaaacacctataaaagatggtaaagtaaaatgcagaataatgaaggttcaaagtgatagtacatttacaaaagaagaaagtgattttgtagaagtgaaatactggaggctttcttttgaaacttctaagtatgcacttattacggtttttaatagatatgacaatactaaaaaaagatcttatggacttgtttcaggcagcataatcgaaagaggagcagtttcttctagctatgctcatgaccatcataatttaatggtaatgggaagaaatacaaaagatatgaaagtagcagcaaattgggttattaaaaacaatgggggttactgtgtagctttggatgggaaaattatagcaagtcttgctcttcccgttggtggtatattgtctgaagaaccaatcgaatccatagcttcaaatttaaaagaagtgagaatggcattaaagaaacttggatataaacataataatgaaataatgtcttttagtacactatctcttccagtaagcccagaattaaaaattacggataaaggtcttataagagttaatacacaggaaatagtaagcctattttgtgagtaa

SEQ ID NO: 78 - DNA, 1767 bp, Clostridium autoethanogenum
atgaatccatatgctgatgaacttattttttcagatgtcttatttacatttaagaaaggtgatcatatgaaagtagatataatcataaaagatgtttatatttttaatgcttattttaaaaagtttgtgtatggagatgtgtcaatattaaatggaaggtttttatatatagggaaggattgctataaaaaaataagtgcaagaaagattatagaaggtaacaataaatatctaatacctggccttattgatatacatatgcatattgaaagttcaatgactacaccaacaaactttgcaaatgctgtcattaaaaatggagttacaacagttgttgctgatccacatgaaattgcaaatgtatttggaatagaaggcataaaagctatgataaattctgataggcagcatataatggatatattttatggcattccaagttctgtaccatcaacatcatctaaacttgaaactacaggtggacaaatagggataaaggaagtatcagaacttataaaaaatgaccgtatatggtgtattggtgaagttatgaattttaaggatttagtttatagtgaagattcacttataaataatataattaaattgataaaaaaagaaaaacctcatttcaaaatagaaggacactgtccaaaaatatcaggatttgatctagcaagatacatatatcaaggggtagatgcagatcatactcagcaaactccaaaatctattgaggaaaagatattaaatggcatgtttttagaaattcaggaaaaatcaatgacaaaagaaaacatggaatatttaatttccaatgatttatatgagcatttttgctttgttactgatgatacaatggcagataaattaattacttcgcatttgaatggacttgttaaaaaagctattgacatgggtatgaagccggaaatggctgtttatgcaagtacttttattccagcaagaagaatgaatttttacgatagaggaagcattgcacctggaaagatagctgattttattttattggatgatttaaaatcatttagtataaatgaagtatataaaaatgggaaactagcatgtaaggatattggagggaaaaaagaacatgaatttccagagaacttttataaaagcataaaaatgaatccagttagtgattcagacttttcagttaaaacacctataaaagatggtaaagtaaaatgcagaataatgaaggttcaaagtgatagtacatttacaaaagaagaaagtgattttgtagaagtgaaatactggaggctttcttttgaaacttctaagtatgcacttattacggtttttaatagatatgacaatactaaaaaaagatcttatggacttgtttcaggcagcataatcgaaagaggagcagtttcttctagctatgctcatgaccatcataatttaatggtaatgggaagaaatacaaaagatatgaaagtagcagcaaattgggttattaaaaacaatgggggttactgtgtagctttggatgggaaaattatagcaagtcttgctcttcccgttggtggtatattgtctgaagaaccaatcgaatccatagcttcaaatttaaaagaagtgagaatggcattaaagaaacttggatataaacataataatgaaataatgtcttttagtacactatctcttccagtaagcccagaattaaaaattacggataaaggtcttataagagttaatacacaggaaatagtaagcctattttgtgagtaa

SEQ ID NO: 79 - protein, 588 aa, Clostridium autoethanogenum
MNPYADELIFSDVLFTFKKGDHMKVDIIIKDVYIFNAYFKKFVYGDVSILNGRFLYIGKDCYKKISARKIIEGNNKYLIPGLIDIHMHIESSMTTPTNFANAVIKNGVTTVVADPHEIANVFGIEGIKAMINSDRQHIMDIFYGIPSSVPSTSSKLETTGGQIGIKEVSELIKNDRIWCIGEVMNFKDLVYSEDSLINNIIKLIKKEKPHFKIEGHCPKISGFDLARYIYQGVDADHTQQTPKSIEEKILNGMFLEIQEKSMTKENMEYLISNDLYEHFCFVTDDTMADKLITSHLNGLVKKAIDMGMKPEMAVYASTFIPARRMNFYDRGSIAPGKIADFILLDDLKSFSINEVYKNGKLACKDIGGKKEHEFPENFYKSIKMNPVSDSDFSVKTPIKDGKVKCRIMKVQSDSTFTKEESDFVEVKYWRLSFETSKYALITVFNRYDNTKKRSYGLVSGSIIERGAVSSSYAHDHHNLMVMGRNTKDMKVAANWVIKNNGGYCVALDGKIIASLALPVGGILSEEPIESIASNLKEVRMALKKLGYKHNNEIMSFSTLSLPVSPELKITDKGLIRVNTQEIVSLFCE

SEQ ID NO: 80 - DNA, 1803 bp, Clostridium autoethanogenum
atgtttaaaaaaaatattacttttaagcttacccttggctttttaacaattgttatcgtttcaactcttttaattggaattattgcattaaatgtatttaaaaacaatatctatgaaatcaaaagaaataatatgaaaaaacatgctttagaaatttcaaatactattaggccttatatgtctcaaagcactaaagaaaaaagttttgtaaatattataaatttaatagactccattgataacgcaaaactttggatactagattccaataaaaatattataagtgcgtcaaataataaagatactgctctcacttacattaacgatgcagatgtaaaaaaaacatacaattctattactaaaaaagtattcactggttcagaaacatacggtgagatatacaacccttactataaagaatacatgatgactacagcagttccaataaaaaatagcagcagcactgtaataggtgcagtaattcttaattcttctttatatgatttatcaaattctatgaacaaattttttatatatatagttttaactcttataggcgaaatatgccttgctggatttatgggatattatttttccaaaaatatatctaaaccattaaaaaaaataaattcatctgcacttgaactggcaagcggtcactatggaataaagacaaatatatatcataaagatgaaattggagaattatcaaattcatttgatttattatctctaaaacttcagtacaccataggcaaactttttgaagaaaaaactaagttaagtaatgtactaacaagtatgagcgagggtattttatctctagatacaaattttcatattataaatattaatcactctaccttagaactgctgtctttaaagaacattgaatcagacacaaaagttaatgaaatattactaaatttaaatataattgaagagtttaattttactatatcaaatgatactaaaaaatctacagtaaaagaatatctaaataaagtattaaatttctctatatctcctataaaaaataatttaaatcaagttataggtggtgtaattcttattcaagatgtaagtgaaaaagaaaaacttgaacaaatgcgaaaagactttatttcaaatgtttcacatgaatttagaactcctttaacagtcataaaaggcaacttagaatctattgtagatggaataactcaaccagaatgtatacaagatacctgtattacactcctaaatgaaactaacaggcttgaaagaatggttaaagatttgcttaatttaagcaaattagaatccggtaaattagagattgattttaatgaactagatatcaatatgctagttaatgacaccatacgaagtattaagcccctaatagatgctaaatccatagatttgcagctatctttacagaataatttaccaccattatttagtgactatgacaagttgaagcaattactcataatatttttagataatggaattaaattttcacaaaataaaggtaagttaaagatatcctcatgttctgataatgaaaatatttgtataactattgaagacaatggaattggtatacccgaagatgaaattcagtatttaggtgaaaaatttttcaaagctgacaaatcaagaacttcaaacgctggtggaactggacttggactttcaattgcaaaaagattagtaaaagttttaaatggacatttttctatagaaagtgaacttacaaaaggaacaagaataactatttcttttccagcaaagtctaaaaatgaggtgaaattatga

SEQ ID NO: 81 - DNA, 1803 bp, Clostridium autoethanogenum
atgtttaaaaaaaatattacttttaagcttacccttggctttttaacaattgttatcgtttcaactcttttaattggaattattgcattaaatgtatttaaaaacaatatctatgaaatcaaaagaaataatatgaaaaaacatgctttagaaatttcaaatactattaggccttatatgtctcaaagcactaaagaaaaaagttttgtaaatattataaatttaatagactccattgataacgcaaaactttggatactagattccaataaaaatattataagtgcgtcaaataataaagatactgctctcacttacattaacgatgcagatgtaaaaaaaacatacaattctattactaaaaaagtattcactggttcagaaacatacggtgagatatacaacccttactataaagaatacatgatgactacagcagttccaataaaaaatagcagcagcactgtaataggtgcagtaattcttaattcttctttatatgatttatcaaattctatgaacaaattttttatatatatagttttaactcttataggcgaaatatgccttgctggatttatgggatattatttttccaaaaatatatctaaaccattaaaaaaaataaattcatctgcacttgaactggcaagaggtcactatggaataaagacaaatatatatcataaagatgaaattggagaattatcaaattcatttgatttattatctctaaaacttcagtacaccataggcaaactttttgaagaaaaaactaagttaagtaatgtactaacaagtatgagcgagggtattttatctctagatacaaattttcatattataaatattaatcactctaccttagaactgctgtctttaaagaacattgaatcagacacaaaagttaatgaaatattactaaatttaaatataattgaagagtttaattttactatatcaaatgatactaaaaaatctacagtaaaagaatatctaaataaagtattaaatttctctatatctcctataaaaaataatttaaatcaagttataggtggtgtaattcttattcaagatgtaagtgaaaaagaaaaacttgaacaaatgcgaaaagactttatttcaaatgtttcacatgaatttagaactcctttaacagtcataaaaggcaacttagaatctattgtagatggaataactcaaccagaatgtatacaagatacctgtattacactcctaaatgaaactaacaggcttgaaagaatggttaaagatttgcttaatttaagcaaattagaatccggtaaattagagattgattttaatgaactagatatcaatatgctagttaatgacaccatacgaagtattaagcccctaatagatgctaaatccatagatttgcagctatctttacagaataatttaccaccattatttagtgactatgacaagttgaagcaattactcataatatttttagataatggaattaaattttcacaaaataaaggtaagttaaagatatcctcatgttctgataatgaaaatatttgtataactattgaagacaatggaattggtatacccgaagatgaaattcagtatttaggtgaaaaatttttcaaagctgacaaatcaagaacttcaaacgctggtggaactggacttggactttcaattgcaaaaagattagtaaaagttttaaatggacatttttctatagaaagtgaacttacaaaaggaacaagaataactatttcttttccagcaaagtctaaaaatgaggtgaaattatga

SEQ ID NO: 82 - protein, 600 aa, Clostridium autoethanogenum
MFKKNITFKLTLGFLTIVIVSTLLIGIIALNVFKNNIYEIKRNNMKKHALEISNTIRPYMSQSTKEKSFVNIINLIDSIDNAKLWILDSNKNIISASNNKDTALTYINDADVKKTYNSITKKVFTGSETYGEIYNPYYKEYMMTTAVPIKNSSSTVIGAVILNSSLYDLSNSMNKFFIYIVLTLIGEICLAGFMGYYFSKNISKPLKKINSSALELASGHYGIKTNIYHKDEIGELSNSFDLLSLKLQYTIGKLFEEKTKLSNVLTSMSEGILSLDTNFHIININHSTLELLSLKNIESDTKVNEILLNLNIIEEFNFTISNDTKKSTVKEYLNKVLNFSISPIKNNLNQVIGGVILIQDVSEKEKLEQMRKDFISNVSHEFRTPLTVIKGNLESIVDGITQPECIQDTCITLLNETNRLERMVKDLLNLSKLESGKLEIDFNELDINMLVNDTIRSIKPLIDAKSIDLQLSLQNNLPPLFSDYDKLKQLLIIFLDNGIKFSQNKGKLKISSCSDNENICITIEDNGIGIPEDEIQYLGEKFFKADKSRTSNAGGTGLGLSIAKRLVKVLNGHFSIESELTKGTRITISFPAKSKNEVKL

SEQ ID NO: 83 - protein, 600 aa, Clostridium autoethanogenum
MFKKNITFKLTLGFLTIVIVSTLLIGIIALNVFKNNIYEIKRNNMKKHALEISNTIRPYMSQSTKEKSFVNIINLIDSIDNAKLWILDSNKNIISASNNKDTALTYINDADVKKTYNSITKKVFTGSETYGEIYNPYYKEYMMTTAVPIKNSSSTVIGAVILNSSLYDLSNSMNKFFIYIVLTLIGEICLAGFMGYYFSKNISKPLKKINSSALELARGHYGIKTNIYHKDEIGELSNSFDLLSLKLQYTIGKLFEEKTKLSNVLTSMSEGILSLDTNFHIININHSTLELLSLKNIESDTKVNEILLNLNIIEEFNFTISNDTKKSTVKEYLNKVLNFSISPIKNNLNQVIGGVILIQDVSEKEKLEQMRKDFISNVSHEFRTPLTVIKGNLESIVDGITQPECIQDTCITLLNETNRLERMVKDLLNLSKLESGKLEIDFNELDINMLVNDTIRSIKPLIDAKSIDLQLSLQNNLPPLFSDYDKLKQLLIIFLDNGIKFSQNKGKLKISSCSDNENICITIEDNGIGIPEDEIQYLGEKFFKADKSRTSNAGGTGLGLSIAKRLVKVLNGHFSIESELTKGTRITISFPAKSKNEVKL

SEQ ID NO: 84 - DNA, 300 bp, Clostridium autoethanogenum
acgtcaaaaatagtgctgtattgtatacatttttatgattttggtaatttttttacagtcttttgtatgtatttaatttaattaaatttattattttctaaaaaaagcatttatgtatgtttgtatatttcattataggtgctatattataaaaaattttataaatatatatttcaatatgtgaatttaatgttataatctaaataagtatttttatatattttaataattttatcattaagtggtatattgttatttttatttattatctttattgttataagttaaataataattggg

SEQ ID NO: 85 - DNA, 300 bp, Clostridium autoethanogenum
acgtcaaaaatagtgctgtattgtatacatttttatgattttggtaatttttttacagtcttttgtatgtatttaatttaattaaatttattattttctaaaaaaagcatttatgtatgtttgtatatttcattataggtgctatattataaaaaattttataaatatatatttcaatatgtgaatttaatgttataatctaaataagtatttttatatattttaataattttatcattaagtggtatattgttatttttatttattgtctttattgttataagttaaataataattggg

SEQ ID NO: 86 - DNA, 660 bp, Clostridium autoethanogenum
gtgaaaaatttgaataacagagaattattaaaaaaatacatacctgtagctaaatttatttctgaaattctaggagataactgcgaggtagtaatacaagatataactactcctaacaattctataatttttataaaaaatggtcatttaacaggaagaaatataggtagtccccttactaacttagttttcaatataattcagaataaaacttatacaaataaaaattacgcagctaactacaaagcagtaggtaattttaaaactttcaaatcttcaagctattttattaaaaatgatgataacaagataataggattactttgtgtaaatatagatgttgaaccttataataaagttaaggatttaatggacaaattaagttttgtatcaaataccgatgtgagcgaaaatgaaaacataaaaacacaagaacaattttatgataatgtggatgatctattgtgtaccatgatcaatgaggctatatctgaaataaatattcttccagaacgcatgtctgctaatgaaaagatttgtgtagtaaaatatttatacgataaaggagctttcaatttgaaaggggctgtagttaaagtagccaaagctctcctggtttccgagcctactatatacaggtatttaaacaaatacatgtaa

SEQ ID NO: 87 - protein, 219 aa, Clostridium autoethanogenum
VKNLNNRELLKKYIPVAKFISEILGDNCEVVIQDITTPNNSIIFIKNGHLTGRNIGSPLTNLVFNIIQNKTYTNKNYAANYKAVGNFKTFKSSSYFIKNDDNKIIGLLCVNIDVEPYNKVKDLMDKLSFVSNTDVSENENIKTQEQFYDNVDDLLCTMINEAISEINILPERMSANEKICVVKYLYDKGAFNLKGAVVKVAKALLVSEPTIYRYLNKYM

SEQ ID NO: 88 - DNA, 300 bp, Clostridium autoethanogenum
ttcaagaccgagagaaacaccgaatagaactataatgatacttagaactatttctttattactagatcaaagcattttagttttgaaaatacattaggagaaaatttaaaaagtttatctaaagatgaaatagtagctcttgttgcagcacttgaagaaggtcaaataactaatactagacttcagatattaataaataaaaactctattgaggtaaataaaatattaagctcattaatagataaaaaattattaaaaacggaagggcagcgaagaggcacaatatatttattatctaat

SEQ ID NO: 89 - DNA, 300 bp, Clostridium autoethanogenum
ttcaagaccgatagaaacaccgaatagaactataatgatacttagaactatttctttattactagatcaaagcattttagttttgaaaatacattaggagaaaatttaaaaagtttatctaaagatgaaatagtagctcttgttgcagcacttgaagaaggtcaaataactaatactagacttcagatattaataaataaaaactctattgaggtaaataaaatattaagctcattaatagataaaaaattattaaaaacggaagggcagcgaagaggcacaatatatttattatctaat

SEQ ID NO: 90 - DNA, 333 bp, Clostridium autoethanogenum
atgtttaatcacagcagttatttaaatgagaactccataaataacagtgaagatatacaaaaaaaattagttgacatagtgaaaaatgtaaaaagagtaaatttaagtcttatggaggatattatattagaattatgtgaagttaaacctataaaattaagtgaattggtaaaattattgaatagaaatgatgtaggtcttagaagtaattatttaaataaaatggaaggcaggacacttaaggaaaccgctgaaatacttaacataaaatatagacatgctatagaaataaaaaaatctgccctaaataaactgaaaaaatatctgaaatag

SEQ ID NO: 91 - protein, 110 aa, Clostridium autoethanogenum
MFNHSSYLNENSINNSEDIQKKLVDIVKNVKRVNLSLMEDIILELCEVKPIKLSELVKLLNRNDVGLRSNYLNKMEGRTLKETAEILNIKYRHAIEIKKSALNKLKKYLK

SEQ ID NO: 92 - DNA, 342 bp, Clostridium autoethanogenum
atggaatttgaaaaggaagttttaaaagggtatatagacacactgatactgtcagttctgtatgaaaatgatatgtacggttatgaaatatcaaagagaattagagaaaagtctaatgatgaatttcaaatgaaagaaactacactatatgtatgtttaaaaagattagaaaagaaaaattacattgaaggctactggaatgatgaaaaaaatactggcgggggaaggaggcgttactataaaattttagaagaaggaaaagagatttttaaagaaaagacaaaagaatggaaggcattacaaaagataatgaaccgtttcgttttagatactgatgagtaa

SEQ ID NO: 93 - DNA, 342 bp, Clostridium autoethanogenum
atggaatttgaaaaggaagttttaaaagggtatatagacacactgatactgtcagttctgtatgaaaatgatatgtacggttatgaaacatcaaagagaattagagaaaagtctaatgatgaatttcaaatgaaagaaactacactatatgtatgtttaaaaagattagaaaagaaaaattacattgaaggctactggaatgatgaaaaaaatactggcgggggaaggaggcgttactataaaattttagaagaaggaaaagagatttttaaagaaaagacaaaagaatggaaggcattacaaaagataatgaaccgtttcgttttagatactgatgagtaa

SEQ ID NO: 94 - protein, 342 aa, Clostridium autoethanogenum
ATGGAATTTGAAAAGGAAGTTTTAAAAGGGTATATAGACACACTGATACTGTCAGTTCTGTATGAAAATGATATGTACGGTTATGAAATATCAAAGAGAATTAGAGAAAAGTCTAATGATGAATTTCAAATGAAAGAAACTACACTATATGTATGTTTAAAAAGATTAGAAAAGAAAAATTACATTGAAGGCTACTGGAATGATGAAAAAAATACTGGCGGGGGAAGGAGGCGTTACTATAAAATTTTAGAAGAAGGAAAAGAGATTTTTAAAGAAAAGACAAAAGAATGGAAGGCATTACAAAAGATAATGAACCGTTTCGTTTTAGATACTGATGAGTAA

SEQ ID NO: 95 - protein, 113 aa, Clostridium autoethanogenum
MEFEKEVLKGYIDTLILSVLYENDMYGYEISKRIREKSNDEFQMKETTLYVCLKRLEKKNYIEGYWNDEKNTGGGRRRYYKILEEGKEIFKEKTKEWKALQKIMNRFVLDTDE

SEQ ID NO: 96 - DNA, 1431 bp, Clostridium autoethanogenum
atggaatatgaattggtaattggcctggaagttcatgtagagctttcgacaaaaactaagatgtattgtggatgcagcactgaatttggaggagaacccaatagtcacgtttgtccaatatgccttgcacttccaggtgcgctccctagaataaataaaaaggtaatagagtataccataaaatcgggaattgcccttaattgctcaataaataaaaagagtagaatggatagaaaaaattatttttatccggattgccctaaaaattatcaaataacccaacacagatttcctttatgtagagatggatacattgaggtagaaaatagtgagggagaaaaaaagaaaataggtattgaaagaatacatatggaagaggacgcagcaaaactaattcatacaaaagcaggtaccttgataaattacaatagagcaggtgtacctcttatagaggtagtgtccaaaccagatataagaaccccagaggaagctacctgttatcttcaaaagttaaaaaatattttgagatccataggagtttcaaattgtaagatggaacaaggttctttaaggtgtgatggtaatatgtctataagacctaaaggcagtagaaaatttggtgtaaaagtggaaataaagaatgtgaattcctttaaagctcttgagaaagcttttgagtatgaatatttaaggcaagtaaaggctgtagagtctggagaaaagatagtacaagaaaccagaagatggaatgagtctaaaaatagaactgaggttatgagggctaaagaacaagctagagattatagatactttccagaaggggatttaacagctataaatatttcaaatgaatggatagaggatataagaaaaactatacctgaacttccttatgataaagtggagagatttactaaggagtacaatgtttcaaaacacgatgctgaggtactaatattaaatatgggggcagcagatttctttgagaaaactgccaagttaagccgaaaccctaaagctgcttttaactggattatgggagatatatcaaggcttatgaatgaaaaatcagtatcattaaatgagcttaaatttaagccggaagacctagtaagtttaatcaaatttatagactcaggtatgatctctaataatataggtaaagaaataatggaggaaatgtttgtaagtggtgcaaaacctgaagaaataattgagaagaggaactttttacaaaatagcagtgaagatgaaattctgcagataataaaaaaggttatgaaagacaatcctaaaccattagaagattataaaaacggtaagaaaagagctattaaatttatgataggtttagttatgaaagaaactaagggaaaggcaaatcctaagattgtaaatgaccttgtaaatagagaatttaataaaaactaa

SEQ ID NO: 97 - DNA, 1431 bp, Clostridium autoethanogenum
atggaatatgaattggtaattggcctggaagttcatgtagagctttcgacaaaaactaagatgtattgtggatgcagcactgaatttggaggagaacccaatagtcacgtttgtccaatatgccttgcacttccaggtgcgctccctagaataaataaaaaggtaatagagtataccataaaaacgggaattgcccttaattgctcaataaataaaaagagtagaatggatagaaaaaattatttttatccggattgccctaaaaattatcaaataacccaacacagatttcctttatgtagagatggatacattgaggtagaaaatagtgagggagaaaaaaagaaaataggtattgaaagaatacatatggaagaggacgcagcaaaactaattcatacaaaagcaggtaccttgataaattacaatagagcaggtgtacctcttatagaggtagtgtccaaaccagatataagaaccccagaggaagctacctgttatcttcaaaagttaaaaaatattttgagatccataggagtttcaaattgtaagatggaacaaggttctttaaggtgtgatggtaatatgtctataagacctaaaggcagtagaaaatttggtgtaaaagtggaaataaagaatgtgaattcctttaaagctcttgagaaagcttttgagtatgaatatttaaggcaagtaaaggctgtagagtctggagaaaagatagtacaagaaaccagaagatggaatgagtctaaaaatagaactgaggttatgagggctaaagaacaagctagagattatagatactttccagaaggggatttaacagctataaatatttcaaatgaatggatagaggatataagaaaaactatacctgaacttccttatgataaagtggagagatttactaaggagtacaatgtttcaaaacacgatgctgaggtactaatattaaatatgggggcagcagatttctttgagaaaactgccaagttaagccgaaaccctaaagctgcttttaactggattatgggagatatatcaaggcttatgaatgaaaaatcagtatcattaaatgagcttaaatttaagccggaagacctagtaagtttaatcaaatttatagactcaggtatgatctctaataatataggtaaagaaataatggaggaaatgtttgtaagtggtgcaaaacctgaagaaataattgagaagaggaactttttacaaaatagcagtgaagatgaaattctgcagataataaaaaaggttatgaaagacaatcctaaaccattagaagattataaaaacggtaagaaaagagctattaaatttatgataggtttagttatgaaagaaactaagggaaaggcaaatcctaagattgtaaatgaccttgtaaatagagaatttaataaaaactaa

SEQ ID NO: 98 - protein, 1431 aa, Clostridium autoethanogenum
ATGGAATATGAATTGGTAATTGGCCTGGAAGTTCATGTAGAGCTTTCGACAAAAACTAAGATGTATTGTGGATGCAGCACTGAATTTGGAGGAGAACCCAATAGTCACGTTTGTCCAATATGCCTTGCACTTCCAGGTGCGCTCCCTAGAATAAATAAAAAGGTAATAGAGTATACCATAAAATCGGGAATTGCCCTTAATTGCTCAATAAATAAAAAGAGTAGAATGGATAGAAAAAATTATTTTTATCCGGATTGCCCTAAAAATTATCAAATAACCCAACACAGATTTCCTTTATGTAGAGATGGATACATTGAGGTAGAAAATAGTGAGGGAGAAAAAAAGAAAATAGGTATTGAAAGAATACATATGGAAGAGGACGCAGCAAAACTAATTCATACAAAAGCAGGTACCTTGATAAATTACAATAGAGCAGGTGTACCTCTTATAGAGGTAGTGTCCAAACCAGATATAAGAACCCCAGAGGAAGCTACCTGTTATCTTCAAAAGTTAAAAAATATTTTGAGATCCATAGGAGTTTCAAATTGTAAGATGGAACAAGGTTCTTTAAGGTGTGATGGTAATATGTCTATAAGACCTAAAGGCAGTAGAAAATTTGGTGTAAAAGTGGAAATAAAGAATGTGAATTCCTTTAAAGCTCTTGAGAAAGCTTTTGAGTATGAATATTTAAGGCAAGTAAAGGCTGTAGAGTCTGGAGAAAAGATAGTACAAGAAACCAGAAGATGGAATGAGTCTAAAAATAGAACTGAGGTTATGAGGGCTAAAGAACAAGCTAGAGATTATAGATACTTTCCAGAAGGGGATTTAACAGCTATAAATATTTCAAATGAATGGATAGAGGATATAAGAAAAACTATACCTGAACTTCCTTATGATAAAGTGGAGAGATTTACTAAGGAGTACAATGTTTCAAAACACGATGCTGAGGTACTAATATTAAATATGGGGGCAGCAGATTTCTTTGAGAAAACTGCCAAGTTAAGCCGAAACCCTAAAGCTGCTTTTAACTGGATTATGGGAGATATATCAAGGCTTATGAATGAAAAATCAGTATCATTAAATGAGCTTAAATTTAAGCCGGAAGACCTAGTAAGTTTAATCAAATTTATAGACTCAGGTATGATCTCTAATAATATAGGTAAAGAAATAATGGAGGAAATGTTTGTAAGTGGTGCAAAACCTGAAGAAATAATTGAGAAGAGGAACTTTTTACAAAATAGCAGTGAAGATGAAATTCTGCAGATAATAAAAAAGGTTATGAAAGACAATCCTAAACCATTAGAAGATTATAAAAACGGTAAGAAAAGAGCTATTAAATTTATGATAGGTTTAGTTATGAAAGAAACTAAGGGAAAGGCAAATCCTAAGATTGTAAATGACCTTGTAAATAGAGAATTTAATAAAAACTAA

SEQ ID NO: 99 - protein, 476 aa, Clostridium autoethanogenum
MEYELVIGLEVHVELSTKTKMYCGCSTEFGGEPNSHVCPICLALPGALPRINKKVIEYTIKSGIALNCSINKKSRMDRKNYFYPDCPKNYQITQHRFPLCRDGYIEVENSEGEKKKIGIERIHMEEDAAKLIHTKAGTLINYNRAGVPLIEVVSKPDIRTPEEATCYLQKLKNILRSIGVSNCKMEQGSLRCDGNMSIRPKGSRKFGVKVEIKNVNSFKALEKAFEYEYLRQVKAVESGEKIVQETRRWNESKNRTEVMRAKEQARDYRYFPEGDLTAINISNEWIEDIRKTIPELPYDKVERFTKEYNVSKHDAEVLILNMGAADFFEKTAKLSRNPKAAFNWIMGDISRLMNEKSVSLNELKFKPEDLVSLIKFIDSGMISNNIGKEIMEEMFVSGAKPEEIIEKRNFLQNSSEDEILQIIKKVMKDNPKPLEDYKNGKKRAIKFMIGLVMKETKGKANPKIVNDLVNREFNKN

SEQ ID NO: 100 - DNA, 1083 bp, Clostridium autoethanogenum
atgaaattcaatgtagcactaataccaggagacggaattggacctgaagctattgaacaagctgttaaagtattagataaagttggagaaatattttcacatgagtttaaatacacaaaagtttatgcaggaggatcagctgttgataaatatgataagccccttccagaagaaactttagatatttgtaaaaaaagcgatgcagttttacttggggcaataggaggagataagtggagcaatcttcctttaaataaaaaaccagaaaaagcacttttgactttgagaaaagaattaggactttatgcaaacttgcgtcctgcagtactgcaaagatcattaaaaaatgattgccctttaaagtctgaacttgtaaaagatggtattgatatgattataataagagaattaactggaggaatgtattctggtgagaaaggagtaaggcagggcaaagatggaaaagaagcttatgatgttgagtgctatagtgaaaatacagttagaagaatagcttcaaaggcttttgaaattgcacttaaaagaagaaaaaaggtaactagtattgataaagctaatgttttagaaagttccatgctttggagaaatactgtaaatgaagtagcaaaaaattaccctgaagttgagttagaacagatatatgtagatagtgcctcaatgaaaatcttaaaaaatcctggtgcatttgatgtagtattggcatcaaatgcatttggggatattatagcagacgaagctagccaaatagttggatctattggaaccttgccagctgcaagccttggggagaaaagcttgggaatgtttgagccaaatcaaatacattcttccgtgcaggatattgctgggaaaaacacggcaaatcctatagctgtaataatgtcttctgccatgatgcttagatactcatttaatttaaatgaagaagcttctgctattgagaaagctgtatcagagacacttgaaaatggttatagaactagagatatgatgactgatggaatgaaacttgttggaactagggaaatgggagatgttatagctaaattgttaagtaagtaa

SEQ ID NO: 101 - DNA, 1083 bp, Clostridium autoethanogenum
atgaaattcaatgtagcactaataccaggagacggaattggacctgaagctattgaacaagctgttaaagtattagataaagttggagaaatattttcacatgagtttaaatacacaaaagtttatgcaggaggatcagctgttgataaatatgataagccccttccagaagaaactttagatatttgtaaaaaaagcgatgcagttttacttggggcaataggaggagataagtggagcaatcttcctttaaataaaaaaccagaaaaagcacttttgactttgagaaaagaattaggactttatgcaaacttgcgtcctgcagtactgcaaagatcattaaaaaatgattgccctttaaagtctgaacttgtaaaagatggtattgatatgattataataagagaattaactggaggaatgtattctggtgagaaaggagtaaggcagggcaaagatggaaaagaagcttatgatgttgagtgctatagtgaaaatacagttagaagaatagcttcaaaggcttttgaaattgcacttaaaagaagaaaaaaggtaactagtattgataaagctaatattttagaaagttccatgctttggagaaatactgtaaatgaagtagcaaaaaattaccctgaagttgagttagaacagatatatgtagatagtgcctcaatgaaaatcttaaaaaatcctggtgcatttgatgtagtattggcatcaaatgcatttggggatattatagcagacgaagctagccaaatagttggatctattggaaccttgccagctgcaagccttggggagaaaagcttgggaatgtttgagccaaatcaaatacattcttccgtgcaggatattgctgggaaaaacacggcaaatcctatagctgtaataatgtcttctgccatgatgcttagatactcatttaatttaaatgaagaagcttctgctattgagaaagctgtatcagagacacttgaaaatggttatagaactagagatatgatgactgatggaatgaaacttgttggaactagggaaatgggagatgttatagctaaattgttaagtaagtaa

SEQ ID NO: 102 - protein, 1083 aa, Clostridium autoethanogenum
ATGAAATTCAATGTAGCACTAATACCAGGAGACGGAATTGGACCTGAAGCTATTGAACAAGCTGTTAAAGTATTAGATAAAGTTGGAGAAATATTTTCACATGAGTTTAAATACACAAAAGTTTATGCAGGAGGATCAGCTGTTGATAAATATGATAAGCCCCTTCCAGAAGAAACTTTAGATATTTGTAAAAAAAGCGATGCAGTTTTACTTGGGGCAATAGGAGGAGATAAGTGGAGCAATCTTCCTTTAAATAAAAAACCAGAAAAAGCACTTTTGACTTTGAGAAAAGAATTAGGACTTTATGCAAACTTGCGTCCTGCAGTACTGCAAAGATCATTAAAAAATGATTGCCCTTTAAAGTCTGAACTTGTAAAAGATGGTATTGATATGATTATAATAAGAGAATTAACTGGAGGAATGTATTCTGGTGAGAAAGGAGTAAGGCAGGGCAAAGATGGAAAAGAAGCTTATGATGTTGAGTGCTATAGTGAAAATACAGTTAGAAGAATAGCTTCAAAGGCTTTTGAAATTGCACTTAAAAGAAGAAAAAAGGTAACTAGTATTGATAAAGCTAATGTTTTAGAAAGTTCCATGCTTTGGAGAAATACTGTAAATGAAGTAGCAAAAAATTACCCTGAAGTTGAGTTAGAACAGATATATGTAGATAGTGCCTCAATGAAAATCTTAAAAAATCCTGGTGCATTTGATGTAGTATTGGCATCAAATGCATTTGGGGATATTATAGCAGACGAAGCTAGCCAAATAGTTGGATCTATTGGAACCTTGCCAGCTGCAAGCCTTGGGGAGAAAAGCTTGGGAATGTTTGAGCCAAATCAAATACATTCTTCCGTGCAGGATATTGCTGGGAAAAACACGGCAAATCCTATAGCTGTAATAATGTCTTCTGCCATGATGCTTAGATACTCATTTAATTTAAATGAAGAAGCTTCTGCTATTGAGAAAGCTGTATCAGAGACACTTGAAAATGGTTATAGAACTAGAGATATGATGACTGATGGAATGAAACTTGTTGGAACTAGGGAAATGGGAGATGTTATAGCTAAATTGTTAAGTAAGTAA

SEQ ID NO: 103 - protein, 360 aa, Clostridium autoethanogenum
MKFNVALIPGDGIGPEAIEQAVKVLDKVGEIFSHEFKYTKVYAGGSAVDKYDKPLPEETLDICKKSDAVLLGAIGGDKWSNLPLNKKPEKALLTLRKELGLYANLRPAVLQRSLKNDCPLKSELVKDGIDMIIIRELTGGMYSGEKGVRQGKDGKEAYDVECYSENTVRRIASKAFEIALKRRKKVTSIDKANVLESSMLWRNTVNEVAKNYPEVELEQIYVDSASMKILKNPGAFDVVLASNAFGDIIADEASQIVGSIGTLPAASLGEKSLGMFEPNQIHSSVQDIAGKNTANPIAVIMSSAMMLRYSFNLNEEASAIEKAVSETLENGYRTRDMMTDGMKLVGTREMGDVIAKLLSK

(19) United States
(12) Patent Application Publication
(10) Pub. No.: US 2015/0210977 A1
(43) Pub. Date: Jul. 30, 2015

(54) BACTERIUM WITH INCREASED TOLERANCE TO BUTYRIC ACIDS

(71) Applicant: LanzaTech New Zealand Limited, Auckland (NZ)

(72) Inventors: Bjorn Daniel Heijstra, Skokie, IL (US); Bakir Al-Sinawi, Sydney (AU); Liam Barry Smith, Auckland (NZ); Rasmus Overgaard Jensen, Skokie, IL (US); Wayne Pierce Mitchell, Auckland (NZ); Michael Koepke, Skokie, IL (US)

(21) Appl. No.: 14/608,151

(22) Filed: Jan. 28, 2015

Related U.S. Application Data
(60) Provisional application No. 61/932,699, filed on Jan. 28, 2014.

(51) Int. Cl. C12N 1/20; C12P 7/62; C12P 7/18; C12P 7/06

(52) U.S. Cl. CPC C12N 1/20; C12P 7/06; C12P 7/62; C12P 7/18

(57) ABSTRACT

The invention provides a bacterium with an increased tolerance to butyric acids, such as 2-hydroxybutyric acid (2-HIBA). In particular, the invention provides a bacterium that tolerates at least 2.5 g/L of butyric acid. The bacterium may be derived, for example, from genus Clostridium, Moorella, Oxobacter, Peptostreptococcus, Acetobacterium, Eubacterium, or Butyribacterium.

### CROSS REFERENCE TO RELATED APPLICATIONS

This application claims the benefit of U.S. Provisional Patent Application 61/932,699 filed Jan. 28, 2014, the entirety of which is incorporated herein by reference.

### SEQUENCE LISTING

This application includes a nucleotide/amino acid sequence listing submitted concurrently herewith and identified as follows: 247,546 byte ASCII (text) file named “LT100US1.txt” created on Jan. 28, 2015, the entirety of which is incorporated herein by reference.

### BACKGROUND OF THE INVENTION

Butyric acids are used in a wide range of industries. For example, butyric acids may be used in the production of biofuels that offer greater sustainability, reduction of greenhouse gas emissions, and security of supply compared to petroleum-based fuels. Additionally, butyric acids may be used in pharmaceutical industries, particularly in prodrug formulations, and in chemical industries for the manufacture of products such as cellulose acetate butyrate plastics.

2-hydroxyisobutyric acid (2-HIB or 2-HIBA) is a particularly valuable butyric acid. At present, 2-HIBA is most commonly produced through isomerization of 3-hydroxybutyric acid (3-HB) and is used as a pharmaceutical intermediate and a complex-forming agent for lanthanide and actinide heavy metals. However, 2-HIBA and derivatives thereof have broad potential applications in polymer synthesis from monomers having an isobutylene carbon skeleton.

During recent years, a number of biosynthetic routes to 2-HIBA and other butyric acids have been explored. However, the growth of many microorganisms are affected by even very low concentrations of butyric acids, which prevents the production of butyric acids in economically viable amounts. Accordingly, there is a strong need for new microorganisms with increased tolerance to butyric acids, particularly 2-HIBA.

### SUMMARY OF THE INVENTION

The invention provides a bacterium with a high tolerance to butyric acids and methods of using the bacterium to produce products.

The bacterium of the invention generally tolerates at least 2.5 g/L of butyric acid, but may tolerate higher levels, such as at least 5 g/L or 10 g/L of butyric acid.

Generally, the bacterium is derived from a parental bacterium that has a lower tolerance to butyric acids. In one embodiment, the bacterium of the invention is derived from a parental bacterium that cannot tolerate at least 2.5 g/L of butyric acid.

In a preferred embodiment, the butyric acid is 2-hydroxyisobutyric acid (2-HIBA).

Certain mutations have been identified in butyric acid tolerant strains. These mutations may be responsible for the observed increase in butyric acid tolerance. In one embodiment, the bacterium of the invention comprises one or more nucleic acid sequences selected from the group consisting of SEQ ID NOs: 2, 6, 10, 14, 17, 21, 24, 25, 29, 32, 36, 40, 44, 47, 51, 55, 58, 62, 66, 70, 74, 78, 81, 85, 89, 93, 97, and 101. In one embodiment, the bacterium of the invention comprises one or more amino sequences selected from the group consisting of SEQ ID NOs: 8, 12, 23, 27, 34, 49, 60, 64, 68, 72, 76, 83, 95, 99, and 103.

The bacterium of the invention may produce a variety of products, including one or more of ethanol, acetate, and 2,3-butanediol.

In one embodiment, the bacterium of the invention is a carboxydotrophic bacterium. In one embodiment, the bacterium of the invention is derived from a bacterium selected from genus Clostridium, Moorella, Oxobacter, Peptostreptococcus, Acetobacterium, Eubacterium, or Butyribacterium. In one embodiment, the bacterium of the invention is derived from Clostridium autoethanogenum, Clostridium ljungdahli, Clostridium carboxidivorans, Clostridium drakei, Clostridium scatologenes, Clostridium aceticum, Clostrdium formicoaceticum, Clostridium magnum, Butyribacterium methyotrphoicum, Acetbacterium woodii, Alkalibaculum bacchi, Blautia producta, Eubacterium limosum, Moorella thermoacetica, Sporomusa ovate, Sporomusa silvacetica, Sporomusa sphaeroides, Oxobacter pfennigii, or Thermoanaerbacter kiuvi. In a preferred embodiment, the bacterium of the invention is derived from Clostridium autoethanogenum or Clostridium ljungdahlii, such as from Clostridium autoethanogenum deposited under DSMZ accession number DSM23693.

The invention further provides a method of producing a product comprising culturing the bacterium of the invention in the presence of a substrate. The product may be, for example, one or more of ethanol, acetate, and 2,3-butanediol. In a preferred embodiment, the substrate comprises one or more of CO, CO₂, and H₂.

### BRIEF DESCRIPTION OF THE DRAWINGS

FIGS. 1A-1D are graphs showing the growth rates of C. autoethanogenum LZ1561 challenged with different concentrations of 2-HIBA in serum bottles. FIG. 1A and FIG. 1B show the respective difference in growth curves for the two sets of growth experiments (n=3). FIG. 1C and FIG. 1D show exponentially fitted lines corresponding to FIG. 1A and FIG. 1B, respectively.

FIG. 2 is a graph depicting the IC₅₀ for C. autoethanogenum LZ1561 challenged with 2-HIBA in serum bottles.

FIGS. 3A-3D are graphs showing the toxicity of 2-HIBA to C. autoethanogenum under continuous fermentation conditions. FIG. 3A and FIG. 3B show the metabolite profile during increased 2-HIBA addition and culture recovery in two parallel continuous fermentations.

FIG. 3C and FIG. 3D show the gas profile of the two parallel fermentations, with hourly measurements of CO, CO₂, and H₂.

FIG. 4 is a graph depicting the metabolite profile (production of acetate, ethanol, and 2,3-butanediol) during increased 2-HIBA addition in continuous fermentation for selection of a tolerant strain of C. autoethanogenum. The 2-HIBA concentrated tolerated by the C. autoethanogenum LZ1561 culture was reproducibly raised from 1.1 g/L to 6.7 g/L (600% increase in tolerance).

FIG. 5 is a graph depicting gas (CO, CO₂, and H₂) uptake during increased 2-HIBA addition in continuous fermentation for selection of a tolerant strain of C. autoethanogenum.

FIG. 6 is a graph showing the growth profile with of C. autoethanogenum LZ1561 and the 2-HIBA tolerant strain with and without 2.2 g/L 2-HIBA challenge.

FIG. 7 is a graph showing the growth rate calculation for of C. autoethanogenum LZ1561 and the 2-HIBA tolerant strain with and without 2.2 g/L 2-HIBA challenge.

FIG. 8 is a graph showing the metabolic profile of the 2-HIBA tolerant strain. In particular, the 2-HIBA tolerant strain appears to produce less 2,3-butanediol than C. autoethanogenum LZ1561.

### DETAILED DESCRIPTION OF THE INVENTION

The invention provides a bacterium that tolerates at least 2.5 g/L of butyric acid. In certain embodiments, the bacterium tolerates at least 2.5 g/L, at least 3.5 g/L, at least 4 g/L, at least 4.5 g/L, at least 5 g/L, at least 5.5 g/L, at least 6 g/L, at least 6.5 g/L, at least 6.7 g/L, at least 7 g/L, at least 7.5 g/L, at least 8 g/L, at least 8.5 g/L, at least 9 g/L, at least 9.5 g/L, or at least 10 g/L.

The butyric acid (butanoic acid) may be any suitable butyric acid or a salt (butyrate), ester (butanoate), isomer, or derivative thereof. Generally, the butyric acid is toxic to wild-type or unadapted microorganisms at relatively low concentrations (e.g., at 1 g/L, 1.5 g/L, or 2 g/L). In one embodiment, the butyric acid is a hydroxybutyric acid, which is a four-carbon organic molecule having both hydroxyl and carboxylic acid functional groups. In another embodiment, the butyric acid is 2-hydroxybutyric acid (alpha-hydroxybutyric acid), 3-hydroxybutyric acid (beta-hydroxybutyric acid), or 4-hydroxybutyric acid (gamma-hydroxybutyric acid). In a particularly preferred embodiment, the butyric acid is 2-hydroxyisobutyric acid (2-HIBA or 2-HIB).

The terms “tolerates,” “tolerance,” “tolerance to,” “tolerant of,” and the like refer to the ability or capacity of the referenced microorganism to grow or survive in the presence of a certain amount of a substance, particularly a toxin. Herein, these terms are generally used to describe the ability or capacity of the referenced microorganism to grow or survive in the presence of a certain amount of butyric acid, such as 2-HIBA. The terms “increased tolerance” or “decreased tolerance” indicate that the referenced microorganism has a higher or lower, respectively, ability or capacity to grow or survive in the presence of a certain substance compared to a wild-type, parental, or non-adapted microorganism. In general, a microorganism that “tolerates” a certain amount of a substance has a growth rate of at least half the maximum growth rate of the microorganism in the presence of that amount of the substance. Tolerance may also be measured in terms of the survival of a microorganism or a population of microorganisms, the growth rate of a microorganism or population of microorganisms, and/or the rate of production of one or more products by a microorganism or population of microorganisms in the presence of butyric acids. The half maximal inhibitory concentration (IC₅₀) is a measure of the effectiveness of a substance in inhibiting a specific biological or biochemical function.

The bacterium of the invention tolerates concentrations of butyric acids that may be toxic to (i.e., not tolerated by) the wild-type, parental, or non-adapted bacterium from which the bacterium of the invention is derived. In one embodiment, the bacterium of the invention is derived from a parental bacterium that cannot tolerate at least 2.5 g/L of butyric acid or at least 5 g/L of butyric acid. In a related embodiment, the bacterium of the invention is derived from a parental bacterium that cannot tolerate at least 2.5 g/L of 2-HIBA or at least 5 g/L of 2-HIBA.

The bacterium of the invention may comprise genetic mutations responsible for the observed increase in tolerance to butyric acids, such as 2-HIBA. For example, the bacterium of the invention may comprise one or more mutations in the genes, genetic elements, or proteins described in Example 5. In one embodiment, the bacterium of the invention comprises one or more nucleic acid sequences selected from the group consisting of SEQ ID NOs: 2, 6, 10, 14, 17, 21, 24, 25, 29, 32, 36, 40, 44, 47, 51, 55, 58, 62, 66, 70, 74, 78, 81, 85, 89, 93, 97, and 101. In one embodiment, the bacterium of the invention comprises one or more amino sequences selected from the group consisting of SEQ ID NOs: 8, 12, 23, 27, 34, 49, 60, 64, 68, 72, 76, 83, 95, 99, and 103.

“Mutated” refers to a nucleic acid or protein that has been modified in the bacterium of the invention compared to the wild-type or parental microorganism from which the bacterium of the invention is derived. In one embodiment, the mutation may be a deletion, insertion, or substitution in a gene encoding an enzyme. In another embodiment, the mutation may be a deletion, insertion, or substitution of one or more amino acids in an enzyme.

The term “genetic modification” broadly refers to manipulation of the genome or nucleic acids of a microorganism. Methods of genetic modification of include heterologous gene expression, gene or promoter insertion or deletion, altered gene expression or inactivation, enzyme engineering, directed evolution, knowledge-based design, random mutagenesis methods, gene shuffling, and codon optimization. Such methods are described, for example, in Sambrook, Molecular Cloning: A Laboratory Manual, Cold Spring Harbor Laboratory Press, Cold Spring Harbor, N.Y., 2001; Pleiss, Curr Opin Biotechnol, 22: 611-617, 2011; Park, Protein Engineering and Design, CRC Press, 2010.

The term “variants” includes nucleic acids and proteins whose sequence varies from the sequence of a reference nucleic acid and protein, such as a sequence of a reference nucleic acid and protein disclosed in the prior art or exemplified herein. The invention may be practiced using variant nucleic acids or proteins that perform substantially the same function as the reference nucleic acid or protein. For example, a variant protein may perform substantially the same function or catalyze substantially the same reaction as a reference protein. A variant gene may encode the same or substantially the same protein as a reference gene. A variant promoter may have substantially the same ability to promote the expression of one or more genes as a reference promoter.

Such nucleic acids or proteins may be referred to herein as “functionally equivalent variants.” By way of example, functionally equivalent variants of a nucleic acid may include allelic variants, fragments of a gene, mutated genes, polymorphisms, and the like. Homologous genes from other microorganisms are also examples of functionally equivalent variants. Functionally equivalent variants also includes nucleic acids whose sequence varies as a result of codon optimization for a particular organism. A functionally equivalent variant of a nucleic acid will preferably have at least approximately 70%, approximately 80%, approximately 85%, approximately 90%, approximately 95%, approximately 98%, or greater nucleic acid sequence identity (percent homology) with the referenced nucleic acid. A functionally equivalent variant of a protein will preferably have at least approximately 70%, approximately 80%, approximately 85%, approximately 90%, approximately 95%, approximately 98%, or greater amino acid identity (percent homology) with the referenced protein. The functional equivalence of a variant nucleic acid or protein may be evaluated using any method known in the art.

A “microorganism” is a microscopic organism, especially a bacterium, archea, virus, or fungus. The microorganism of the invention is typically a bacterium. As used herein, recitation of “microorganism” should be taken to encompass “bacterium.”

A “parental microorganism” is a microorganism used to generate a bacterium of the invention. The parental microorganism may be a naturally-occurring microorganism (i.e., a wild-type microorganism) or a microorganism that has been previously modified (i.e., a mutant or recombinant microorganism). The bacterium of the invention may be modified to express or overexpress one or more enzymes that were not expressed or overexpressed in the parental microorganism. Similarly, the bacterium of the invention may be modified to contain one or more genes that were not contained by the parental microorganism. In one embodiment, the parental organism is Clostridium autoethanogenum, Clostridium ljungdahlii, or Clostridium ragsdalei. In a preferred embodiment, the parental organism is Clostridium autoethanogenum LZ1561, which is deposited under DSMZ accession number DSM23693.

The term “derived from” indicates that a nucleic acid, protein, or microorganism is modified or adapted from a different (e.g., a parental or wild-type) nucleic acid, protein, or microorganism, so as to produce a new nucleic acid, protein, or microorganism. Such modifications or adaptations typically include insertion, deletion, mutation, or substitution of nucleic acids or genes. Generally, the bacterium of the invention is derived from a parental microorganism. In one embodiment, the bacterium of the invention is derived from Clostridium autoethanogenum, Clostridium ljungdahli, Clostridium carboxidivorans, Clostridium drakei, Clostridium scatologenes, Clostridium aceticum, Clostrdium formicoaceticum, Clostridium magnum, Butyribacterium methyotrphoicum, Acetbacterium woodii, Alkalibaculum bacchi, Blautia producta, Eubacterium limosum, Moorella thermoacetica, Sporomusa ovate, Sporomusa silvacetica, Sporomusa sphaeroides, Oxobacter pfennigii, or Thermoanaerbacter kiuvi. In a preferred embodiment, the bacterium of the invention is derived from Clostridium autoethanogenum or Clostridium ljungdahlii. For example, the bacterium of the invention may derived from Clostridium autoethanogenum having the identifying characteristics of the strain deposited under DSMZ accession number DSM1006, DSM19630, or DSM23693. In a particularly preferred embodiment, the bacterium of the invention is derived from Clostridium autoethanogenum deposited under DSMZ accession number DSM23693.

A “carboxydotroph” is a microorganism capable of tolerating a high concentration of carbon monoxide (CO). The bacterium of the invention may be a carboxydotroph. In one embodiment, the bacterium of the invention is derived from a carboxydotrophic bacterium selected from genus Clostridium, Moorella, Oxobacter, Peptostreptococcus, Acetobacterium, Eubacterium, or Butyribacterium.

The bacterium of the invention may be derived from the cluster of carboxydotrophic Clostridia comprising the species Clostridium autoethanogenum, Clostridium ljungdahlii, Clostridium ragsdalei, and related isolates, including, but not limited to, strains Clostridium autoethanogenum JAI-1T (DSM10061) (Abrini, Arch Microbiol, 161: 345-351, 1994), Clostridium autoethanogenum LBS1560 (DSM19630) (WO 2009/064200), Clostridium autoethanogenum LZ1561 (DSM23693), Clostridium ljungdahlii PETCT (DSM13528=ATCC 55383) (Tanner, Int J Syst Bacteriol, 43: 232-236, 1993), Clostridium ljungdahlii ERI-2 (ATCC 55380) (U.S. Pat. No. 5,593,886), Clostridium ljungdahlii C-01 (ATCC 55988) (U.S. Pat. No. 6,368,819), Clostridium ljungdahlii O-52 (ATCC 55989) (U.S. Pat. No. 6,368,819), Clostridium ragsdalei P11T (ATCC BAA-622) (WO 2008/028055), related isolates such as “Clostridium coskatii” (U.S. Publication 2011/0229947), or mutated strains such as Clostridium ljungdahlii OTA-1 (Tirado-Acevedo, Production of Bioethanol from Synthesis Gas Using Clostridium ljungdahlii, PhD thesis, North Carolina State University, 2010).

These strains form a subcluster within the Clostridial rRNA cluster I and their 16S rRNA gene is more than 99% identical with a similar low GC content of around 30%. However, DNA-DNA reassociation and DNA fingerprinting experiments showed that these strains belong to distinct species (WO 2008/028055). The strains of this cluster are defined by common characteristics, having both a similar genotype and phenotype, and they all share the same mode of energy conservation and fermentative metabolism. Furthermore, the strains of this cluster lack cytochromes and conserve energy via an Rnf complex. All species of this cluster have a similar morphology and size (logarithmic growing cells are between 0.5-0.7×3-5 μm), are mesophilic (optimal growth temperature between 30-37° C.), and are strictly anaerobic (Abrini, Arch Microbiol, 161: 345-351, 1994; Tanner, Int J Syst Bacteriol, 43: 232-236, 1993; and WO 2008/028055). Moreover, they all share the same major phylogenetic traits, such as same pH range (pH 4-7.5, with an optimal initial pH of 5.5-6), strong autotrophic growth on CO-containing gases with similar growth rates, and a similar metabolic profile with ethanol and acetic acid as main fermentation end products, and small amounts of 2,3-butanediol and lactic acid formed under certain conditions (Abrini, Arch Microbiol, 161: 345-351, 1994; Köpke, Curr Opin Biotechnol, 22: 320-325, 2011; Tanner, Int J Syst Bacteriol, 43: 232-236, 1993; and WO 2008/028055). Indole production was observed with all three species as well.

However, the species differentiate in substrate utilization of various sugars (e.g., rhamnose, arabinose), acids (e.g., gluconate, citrate), amino acids (e.g., arginine, histidine), or other substrates (e.g., betaine, butanol). Moreover some of the species were found to be auxotrophic to certain vitamins (e.g., thiamine, biotin) while others were not. The organization and number of Wood-Ljungdahl pathway genes, responsible for gas uptake, has been found to be the same in all species, despite differences in nucleic and amino acid sequences (Köpke, Curr Opin Biotechnol, 22: 320-325, 2011). Also, reduction of carboxylic acids into their corresponding alcohols has been shown in a range of these microorganisms (Perez, Biotechnol Bioeng, 110:1066-1077, 2012). These traits are therefore not specific to one microorganism, like Clostridium autoethanogenum or Clostridium ljungdahlii, but rather general traits for carboxydotrophic, ethanol-synthesizing Clostridia and it can be anticipated that mechanisms work similarly across these strains, although there may be differences in performance.

An “acetogen” is a microorganism that generates or is capable of generating acetate as a product of anaerobic respiration. Typically, acetogens are obligately anaerobic bacteria that use the Wood-Ljungdahl pathway as their main mechanism for energy conservation and for synthesis of acetyl-CoA and acetyl-CoA-derived products, such as acetate (Ragsdale, Biochim Biophys Acta, 1784: 1873-1898, 2008). In one embodiment, the bacterium of the invention is an acetogen.

The bacterium of the invention may produce or be engineered to produce, for example, ethanol (WO 2007/117157), acetate (WO 2007/117157), butanol (WO 2008/115080 and WO 2012/053905), butyrate (WO 2008/115080), 2,3-butanediol (WO 2009/151342), lactate (WO 2011/112103), butene (WO 2012/024522), butadiene (WO 2012/024522), methyl ethyl ketone (2-butanone) (WO 2012/024522 and WO 2013/185123), ethylene (WO 2012/026833), acetone (WO 2012/115527), isopropanol (WO 2012/115527), lipids (WO 2013/036147), 3-hydroxypropionate (3-HP) (WO 2013/180581), isoprene (WO 2013/180584), fatty acids (WO 2013/191567), 2-butanol (WO 2013/185123), 1,2-propanediol (WO 2014/0369152), and 1-propanol (WO 2014/0369152).

The bacterium of the invention may also have a different metabolic profile from the wild-type, parental, or non-adapted bacterium from which the bacterium of the invention is derived. In particular, the bacterium of the invention may produce different products or amounts of products. In one embodiment, the bacterium of the invention produces a comparatively lower amount of 2,3-butanediol compared to the wild-type, parental, or non-adapted bacterium from which the bacterium of the invention is derived. For example, the bacterium of the invention may produce less than about 6 g/L, 5 g/L, 4 g/L, 3 g/L, 2 g/L, or 1 g/L 2,3-butanediol.

The term “substrate” refers to a carbon and/or energy source for the bacterium of the invention. Typically, the substrate is a gaseous substrate that comprises carbon monoxide (CO). The substrate may comprise a major proportion of CO, such as about 20% to 100%, 20% to 70%, 30% to 60%, or 40% to 55% CO by volume. In particular embodiments, the substrate comprises about 25%, 30%, 35%, 40%, 45%, 50%, 55%, or 60% CO by volume. The bacterium of the invention generally converts at least a portion of the CO in the substrate to a product.

While it is not necessary for the substrate to contain any hydrogen (H₂), the presence of H₂ should not be detrimental to product formation and may result improved overall efficiency. For example, in particular embodiments, the substrate may comprise an approximate ratio of H₂:CO of 2:1, 1:1, or 1:2. In one embodiment, the substrate comprises less than about 30%, 20%, 15%, or 10% H₂ by volume. In other embodiments, the substrate comprises low concentrations of H₂, for example, less than 5%, less than 4%, less than 3%, less than 2%, or less than 1% H₂. In further embodiments, the substrate contains substantially no H₂. The substrate may also contain carbon dioxide (CO₂), for example, about 1% to 80% or 1% to 30% CO₂ by volume. In one embodiment, the substrate comprises less than about 20% CO₂ by volume. In further embodiments, the substrate comprises less than about 15%, 10%, or 5% CO₂ by volume. In another embodiment, the substrate contains substantially no CO₂.

Although the substrate is typically gaseous, the substrate may also be provided in alternative forms. For example, the substrate may be dissolved in a liquid saturated with a CO-containing gas using a microbubble dispersion generator (Hensirisak, Appl Biochem Biotechnol, 101: 211-227, 2002). By way of further example, the substrate may be adsorbed onto a solid support.

The substrate may be a waste gas obtained as a by-product of an industrial process or from some other source, such as from automobile exhaust fumes or biomass gasification. In certain embodiments, the industrial process is selected from the group consisting of ferrous metal products manufacturing, such as a steel mill manufacturing, non-ferrous products manufacturing, petroleum refining processes, coal gasification, electric power production, carbon black production, ammonia production, methanol production, and coke manufacturing. In these embodiments, the CO-containing gas may be captured from the industrial process before it is emitted into the atmosphere, using any convenient method. The CO may be a component of syngas, i.e., a gas comprising carbon monoxide and hydrogen. The CO produced from industrial processes is normally flared off to produce CO₂ and therefore the invention has particular utility in reducing CO₂ greenhouse gas emissions. The composition of the substrate may have a significant impact on the efficiency and/or cost of the reaction. For example, the presence of oxygen (O₂) may reduce the efficiency of an anaerobic fermentation process. Depending on the composition of the substrate, it may be desirable to treat, scrub, or filter the substrate to remove any undesired impurities, such as toxins, undesired components, or dust particles, and/or increase the concentration of desirable components.

The bacterium of the invention may be cultured. Typically, the culture is performed in serum bottles or a bioreactor. The term “bioreactor” includes a culture/fermentation device consisting of one or more vessels, towers, or piping arrangements, such as a continuous stirred tank reactor (CSTR), immobilized cell reactor (ICR), trickle bed reactor (TBR), bubble column, gas lift fermenter, static mixer, or other vessel or other device suitable for gas-liquid contact. In some embodiments, the bioreactor may comprise a first growth reactor and a second culture/fermentation reactor. The substrate may be provided to one or both of these reactors. As used herein, the terms “culture” and “fermentation” are used interchangeably. These terms encompass both the growth phase and product biosynthesis phase of the culture/fermentation process.

The culture is generally maintained in an aqueous culture medium that contains nutrients, vitamins, and/or minerals sufficient to permit growth of the bacterium. Preferably the aqueous culture medium is a minimal anaerobic microbial growth medium. Suitable media are known in the art and described, for example, in U.S. Pat. No. 5,173,429, U.S. Pat. No. 5,593,886, and WO 2002/008438.

The culture/fermentation should desirably be carried out under appropriate conditions for production of the target product. Reaction conditions to consider include pressure (or partial pressure of CO), temperature, gas flow rate, liquid flow rate, media pH, media redox potential, agitation rate (if using a continuous stirred tank reactor), inoculum level, maximum gas substrate concentrations to ensure that CO in the liquid phase does not become limiting, and maximum product concentrations to avoid product inhibition. In particular, the rate of introduction of the CO-containing substrate may be controlled to ensure that the concentration of CO in the liquid phase does not become limiting, since products may be consumed by the culture under CO-limited conditions.

Operating a bioreactor at elevated pressures allows for an increased rate of CO mass transfer from the gas phase to the liquid phase. Accordingly, it is generally preferable to perform the culture/fermentation at pressures higher than atmospheric pressure. Also, since a given CO conversion rate is, in part, a function of the substrate retention time and retention time dictates the required volume of a bioreactor, the use of pressurized systems can greatly reduce the volume of the bioreactor required and, consequently, the capital cost of the culture/fermentation equipment. According to examples in U.S. Pat. No. 5,593,886, reactor volume can be reduced in linear proportion to increases in reactor operating pressure. In other words, a bioreactor operated at 10 atmospheres of pressure need only be one tenth the volume of a bioreactor operated at 1 atmosphere of pressure. Additionally, WO 2002/008438 describes gas-to-ethanol fermentations performed under pressures of 30 psig and 75 psig, giving ethanol productivities of 150 g/L/day and 369 g/L/day, respectively. In contrast, fermentations performed using similar media and input gas compositions at atmospheric pressure were found to produce between 10 and 20 times less ethanol per litre per day.

### EXAMPLES

The following examples further illustrate the invention but, of course, should not be construed to limit its scope in any way.

### Example 1

This example demonstrates the general growth of strains of Clostridium.

Clostridium strains were grown at 37° C. in PETC media at pH 5.6 using standard anaerobic techniques (Hungate, Meth Microbiol, 3B: 117-132, 1969; Wolfe, Adv Microb Physiol, 6: 107-146, 1971). Fructose (heterotrophic growth) or 30 psi CO-containing steel mill gas (collected from New Zealand Steel site in Glenbrook, NZ; composition: 44% CO, 32% N₂, 22% CO₂, 2% H₂) in the headspace (autotrophic growth) was used as substrate. For solid media, 1.2% bacto agar (BD, Frankton Lakes, N.J. 07417, USA) was added.

| PETC media component | Concentration per 1.0 L of media | |
|---|---|---|
| NH₄Cl | 1 | g |
| KCl | 0.1 | g |
| MgSO₄•7H₂O | 0.2 | g |
| NaCl | 0.8 | g |
| KH₂PO₄ | 0.1 | g |
| CaCl₂ | 0.02 | g |
| Trace metal solution | 10 | ml |
| Wolfe's vitamin solution | 10 | ml |
| Yeast extract (optional) | 1 | g |
| Resazurin (2 g/L stock) | 0.5 | ml |
| NaHCO₃ | 2 | g |
| Reducing agent | 0.006-0.008% | (v/v) |
| Fructose (for heterotrophic growth) | 5 | g |

### Example 2

This example demonstrates the toxicity of 2-HIBA to C. autoethanogenum LZ1561 in serum bottles and continuous stirred tank reactors (CSTRs).

Serum Bottles

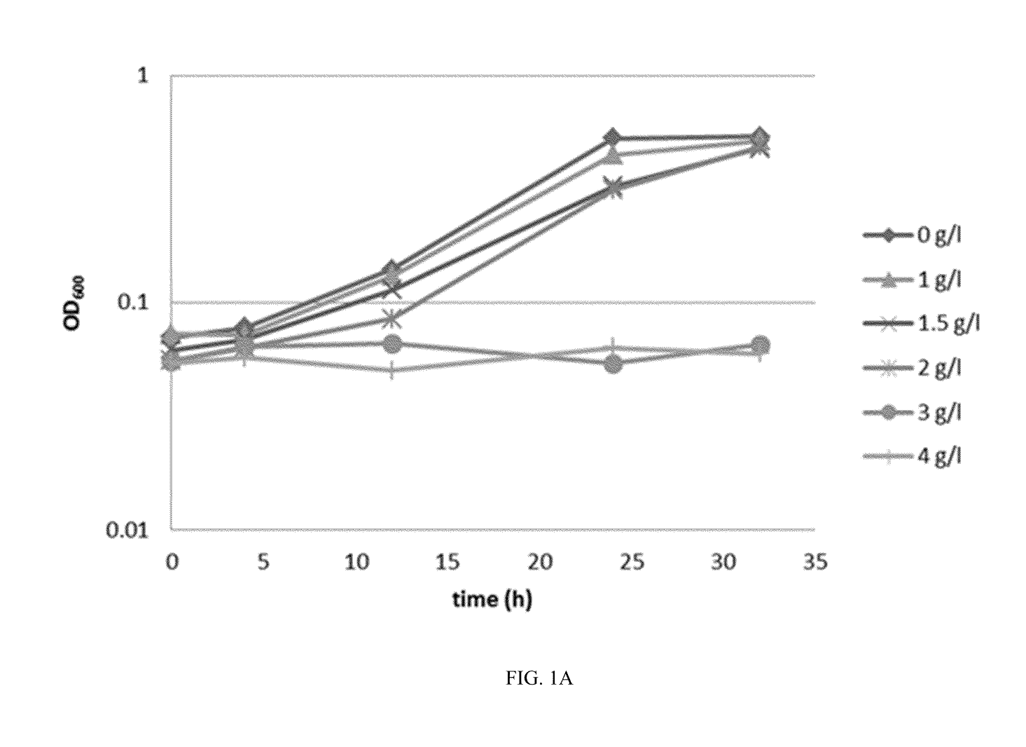

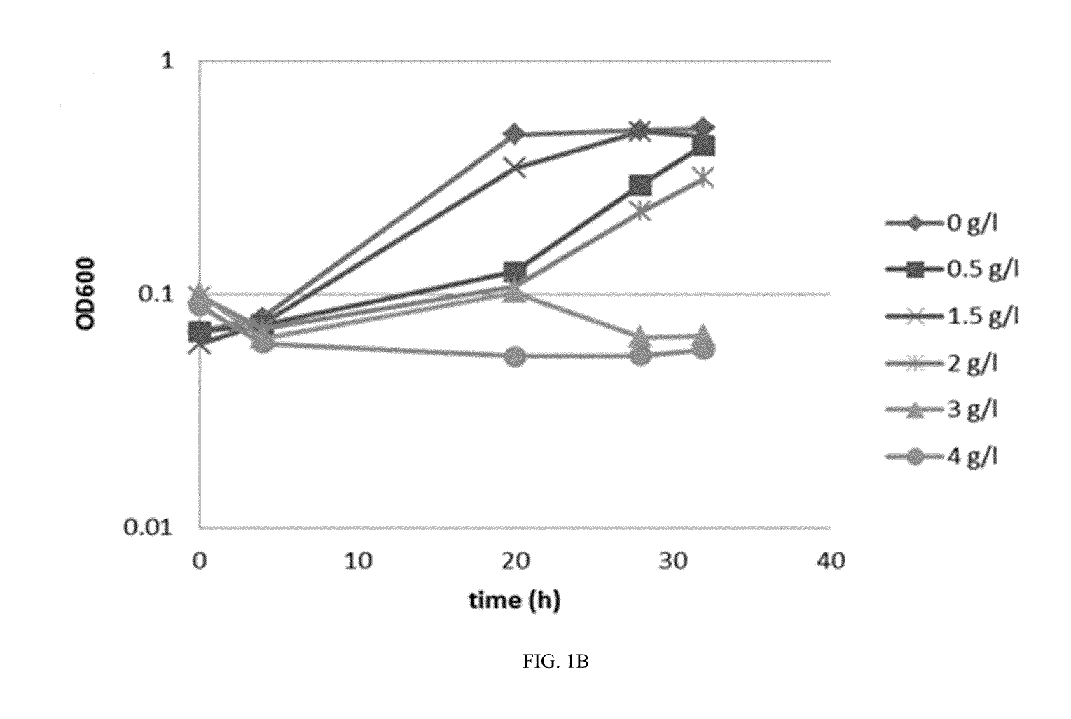

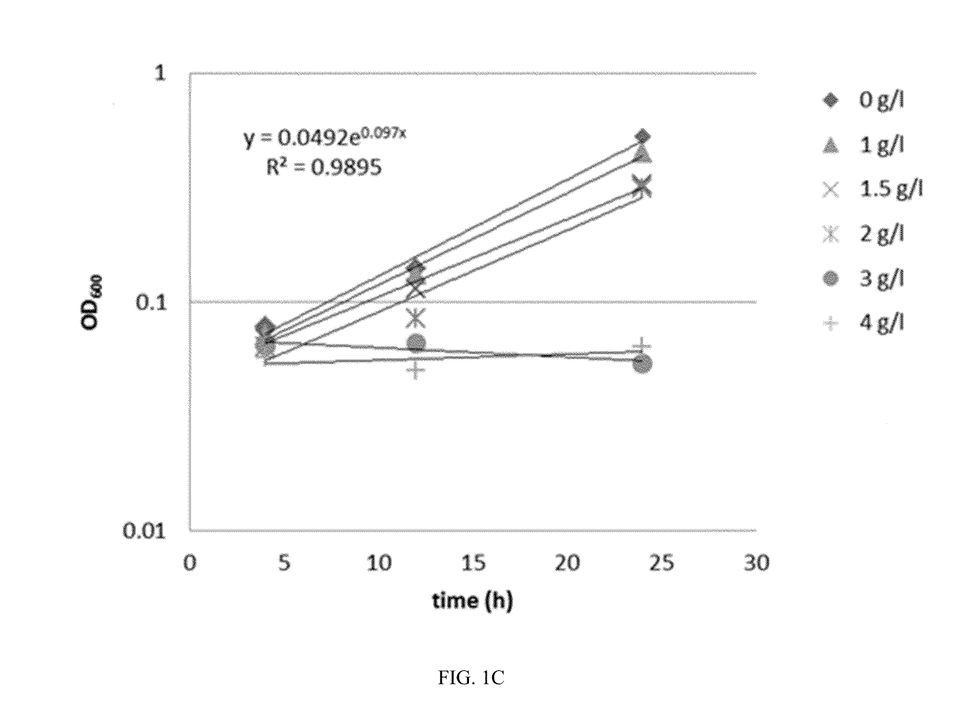

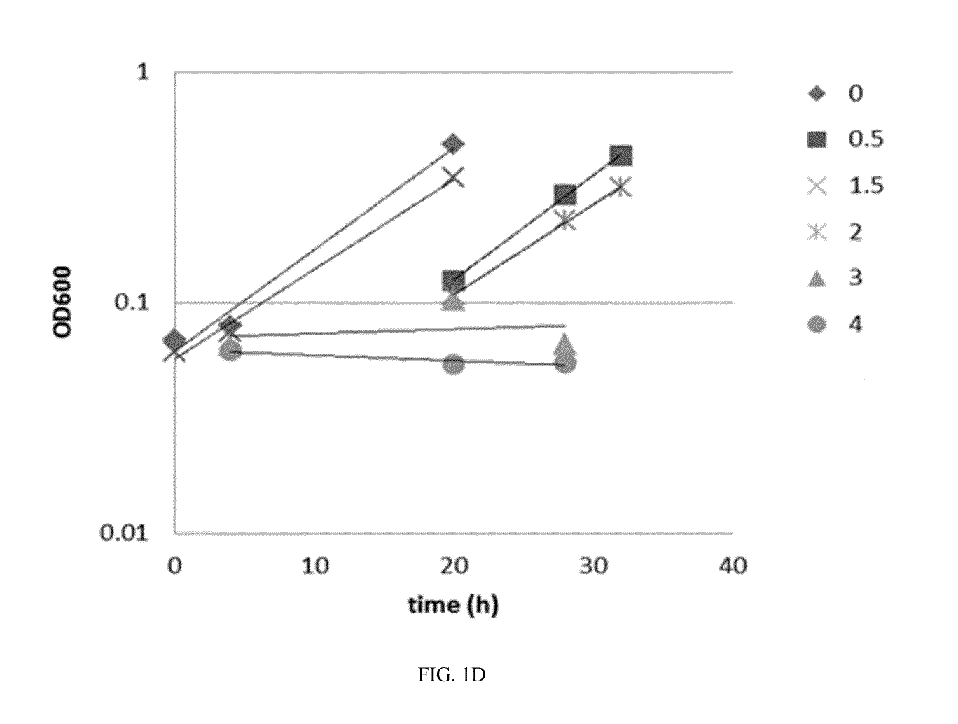

Two sets of serum bottle experiments were performed with media containing 0, 0.5, 1, 1.5, 2, 3, or 4 g/L of 2-HIBA. An average of optical density (OD) data from both sets of growth experiments was plotted. Data points outside the visually observed exponential growth phase were removed and the growth rate (g) for each concentration of 2-HIBA was calculated by fitting an exponential trend line (FIG. 1).

FIGS. 1A-1D show the growth rates of C. autoethanogenum challenged with different concentrations of 2-HIBA. FIG. 1A and FIG. 1B show the respective difference in growth curves for the two sets of growth experiments (n=3). FIG. 1C and FIG. 1D show exponentially fitted lines corresponding to FIG. 1A and FIG. 1B, respectively. FIG. 1C further shows an example of the extracted equation and R² value. To extract the growth rate from the trend line equations, the following equation was used OD=OD₀Xe^{μt}, where OD=y, t=x in the trend line equations, providing the calculated growth rates listed below.

Calculated Growth Rates and R² Values

| | | Set 1 | | Set 2 | |
|---|---|---|---|---|---|
| [2-HIBA] (g/L) | equation example | R² | μ | R² | μ |
| 0 | y = 0.0492e^{0.097x} | 0.989 | 0.097 | 0.98 | 0.101 |
| 0.5 | y = 0.0487e^{0.0963x} | 0.999 | 0.096 | 1 | 0.104 |
| 1 | y = 0.0472e^{0.0923x} | 0.993 | 0.092 | 0.999 | 0.105 |
| 1.5 | y = 0.0482e^{0.0781x} | 0.993 | 0.078 | 0.992 | 0.089 |
| 2 | y = 0.0398e^{0.0821x} | 0.945 | 0.082 | 0.993 | 0.090 |
| 3 | y = 0.0693e0^{-0.009x} | 0.317 | 0.001 | 0.042 | 0.004 |
| 4 | y = 0.0523e^{0.006x} | 0.171 | 0.004 | 0.8589 | −0.005 |

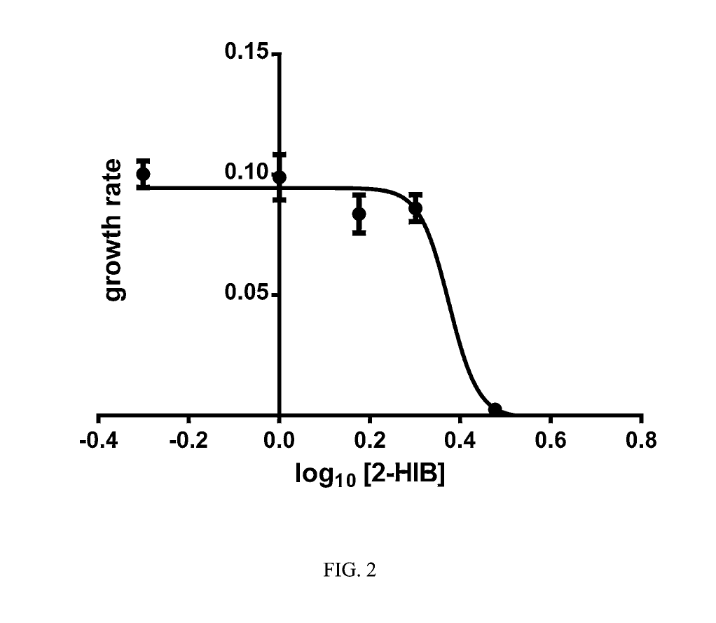

The growth rates (μ) were plotted in Prism6 (GraphPad, USA) as μ vs log₁₀ [2-HIBA] to determine the concentration of 2-HIBA at which the growth rate of C. autoethanogenum is 50% (FIG. 2). An IC₅₀ of 2.2 g/L of 2-HIBA was calculated for C. autoethanogenum in serum bottles. As is illustrated in FIG. 2, a concentration up to 1 g/L of 2-HIBA appears to have little or no effect on growth, followed by a relatively small effect up to a concentration of 2 g/L. However, the effect of over 2 g/L of 2-HIBA on growth is acute, as shown by the steep drop in the curve.

Continuous Stirred Tank Reactors

CSTRs were inoculated with C. autoethanogenum and brought to a stable optical density (OD₆₀₀ nm) and dilution rate (D˜1.5). For all fermentations in CSTRs, chemically defined media was used containing no yeast extract. Parameters were monitored on an hourly basis, including metabolites (measured by HPLC) and gas composition in/out (measured by GC). Early effects of 2-HIBA on culture metabolism included a measurable reduction in CO and/or H₂ utilization rates, followed by a decline in metabolite production rate.

Two CSTRs were run in parallel and received the same inoculum and media in flow rate and composition. The reactors were turned continuous at day 0.9 with media containing 1.5 g/L 2-HIBA at a dilution rate of 1.5. The media was fed until gas uptake reduction was confirmed after which the culture was recovered using an inflow of fresh media that did not contain 2-HIBA. The process was then repeated on the same culture in one reactor while the other served as a control.

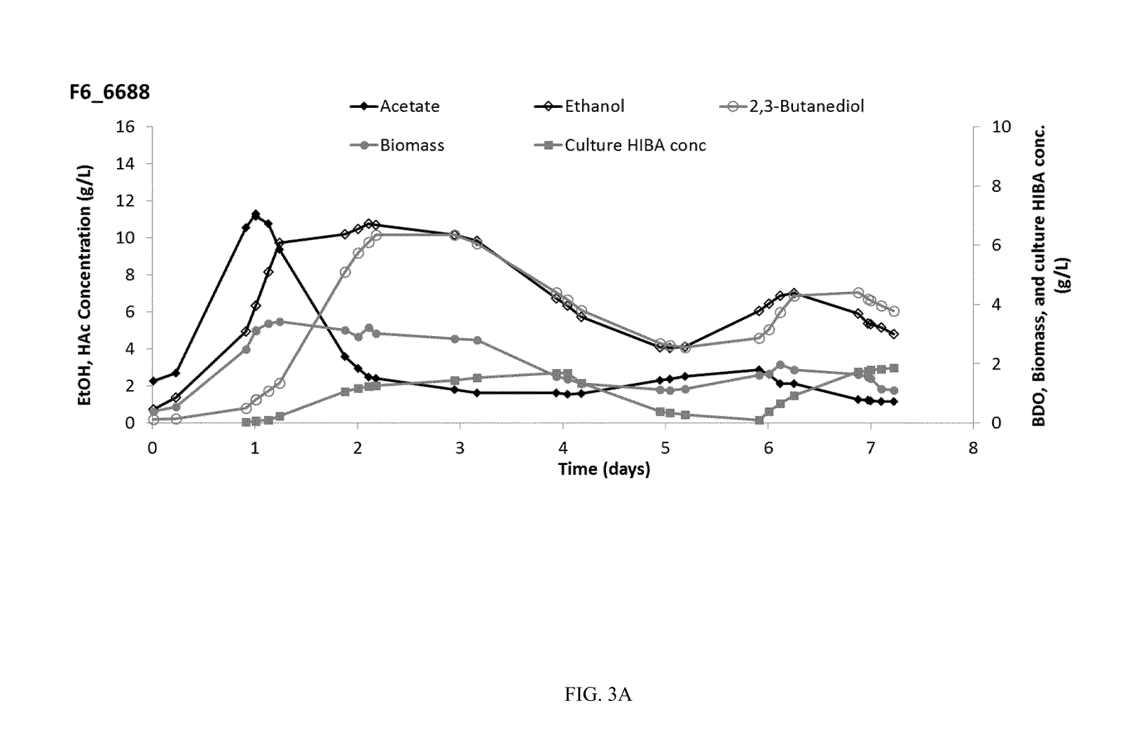

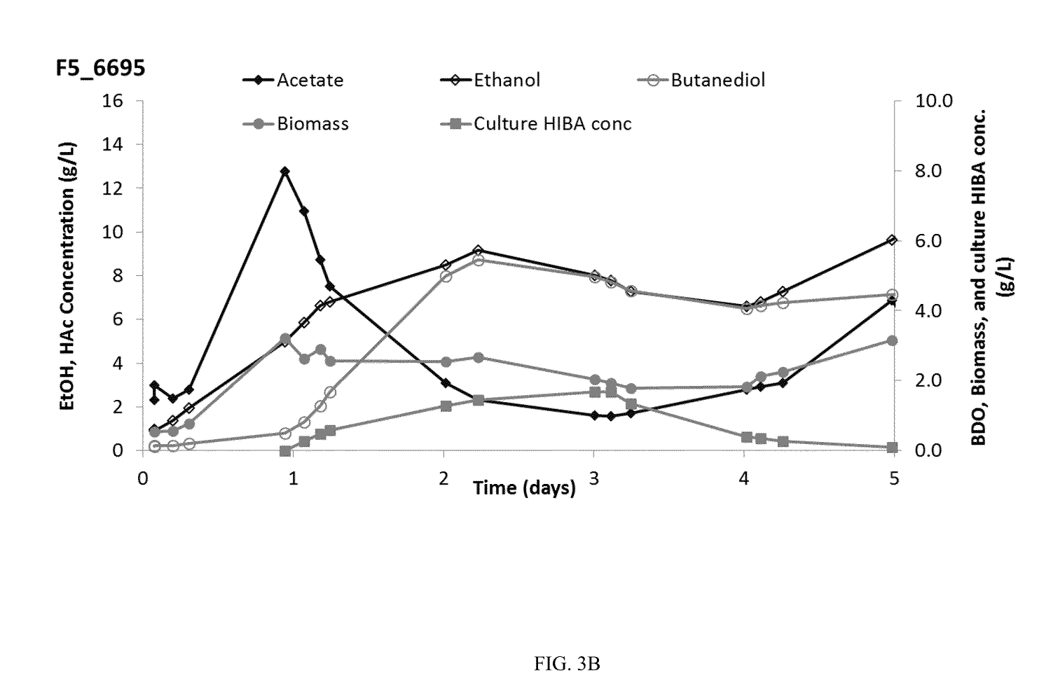

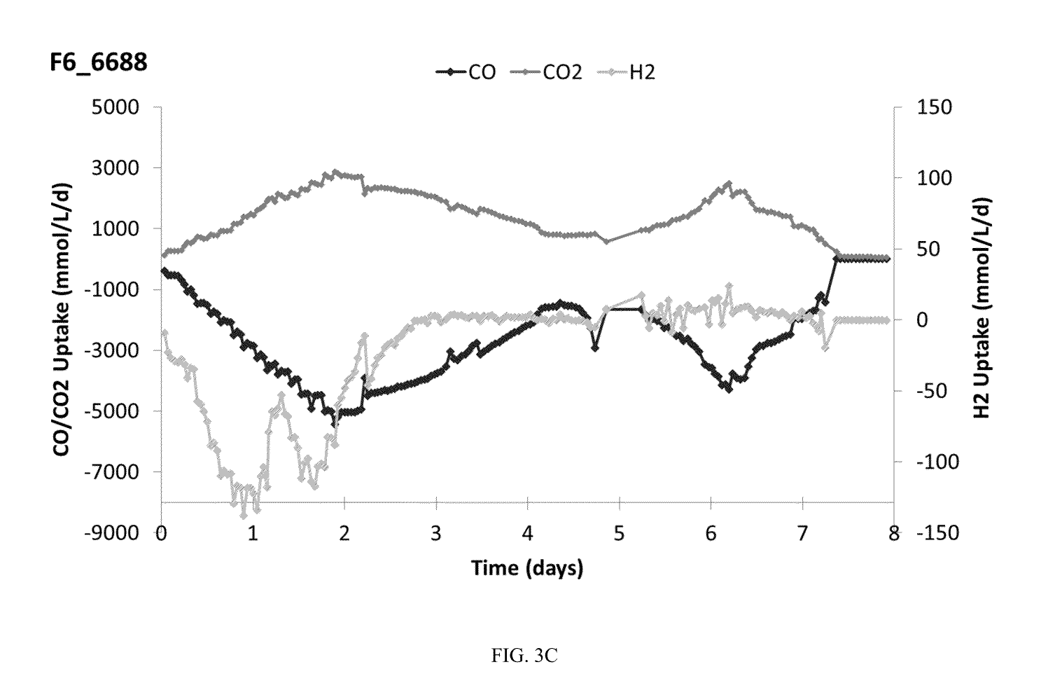

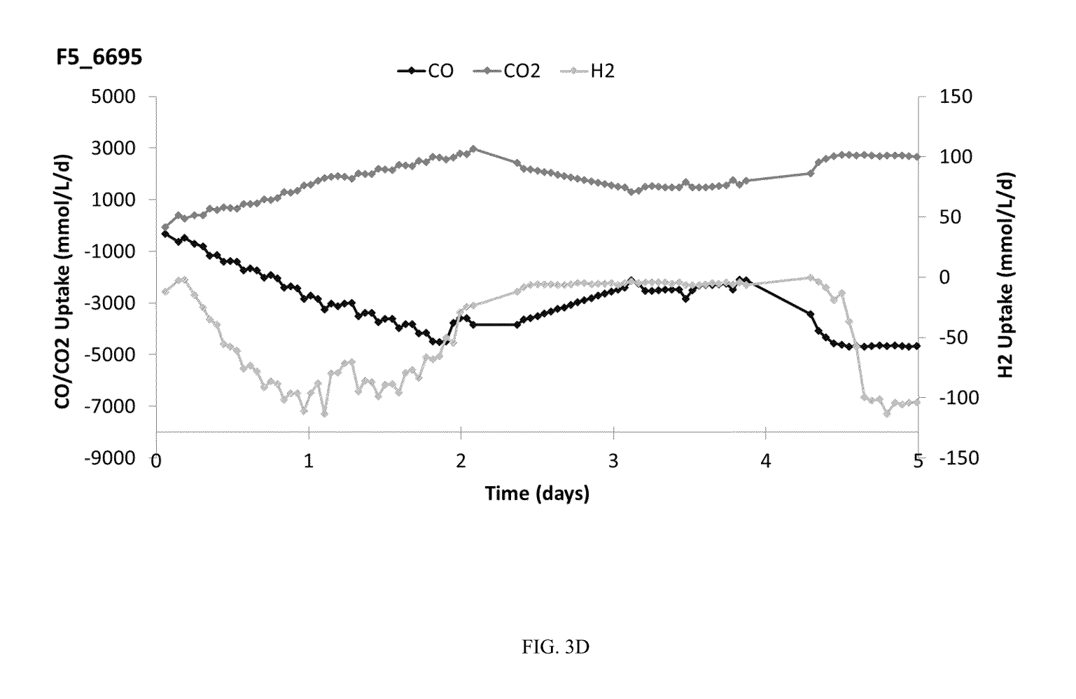

The results of this experiment are illustrated in FIGS. 3A-3D. FIG. 3A and FIG. 3B show the metabolite profile during increased 2-HIBA addition and culture recovery in two parallel continuous fermentations. FIG. 3C and FIG. 3D show the gas profile of the two parallel fermentations, with hourly measurements of CO, CO₂, and H₂.

Following introduction of 2-HIBA, the metabolic profile of C. autoethanogenum shifted to favor 2,3-butanediol production, capping ethanol production. Biomass production was reduced under increasing inflow from 2-HIBA and overall metabolic levels drop caused by reduced CO and H₂ uptake. By removing 2-HIBA from the inflow media, gas uptake and metabolic production is stabilized. This indicates a reversible reaction to 2-HIBA. The average effect level of 2-HIBA in a continuous CSTR system was calculated as 1.15 g/L.

It is important to note that the serum bottle experiments and the CSTR experiments are not directly comparable. The batch-type serum bottle experiments were designed for the purpose of calculating growth rates and IC₅₀, whereas the CSTR experiments were designed to detect the early effects of 2-HIBA on metabolism by continuous monitoring of gas uptake and metabolite production.

### Example 3

This example demonstrates the selection of a 2-HIBA tolerant strain.

Strains were obtained through selection in a continuous fermentation or on agar plates and were tested for increased tolerance to 2-HIBA. Selection in continuous fermentation is most relevant from a process perspective and has shown to be a useful tool to screen for growth-related traits, as only microorganisms that are readily dividing are retained and non-dividing microorganisms are washed out. While this strategy may result in a heterogeneous culture, it can be combined with a selection approach on agar plates, where single colonies guarantee a homogenous culture and differences in colony size are an indicator of growth speed.

Continuous Fermentation

To enhance 2-HIBA tolerance in continuous fermentation, the 2-HIBA concentration in the feeding medium was slowly increased. Microorganisms unable to cope with the increasing 2-HIBA concentration were diluted out from the fermentation system, while microorganisms with improved tolerance were retained. Glycerol stocks were collected as the culture resistance improved.

CSTRs were inoculated with C. autoethanogenum LZ1561. The reactors were started in batch mode and turned to continuous mode at a dilution rate of 1.5 after approximately 40 hours. Once operationally stable, 2-HIBA (1.1 g/L) was added to the feeding media which was run into the reactor at a dilution rate of 1.5. The concentration of 2-HIBA was then slowly increased by approximately 0.05 g/L per day.

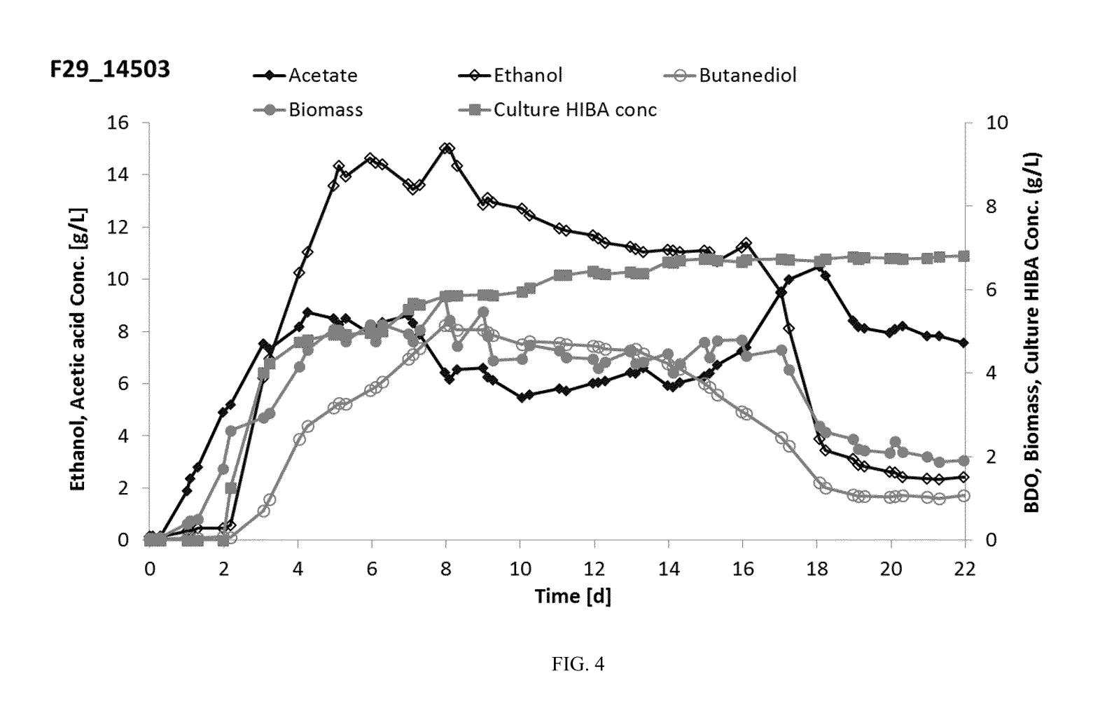

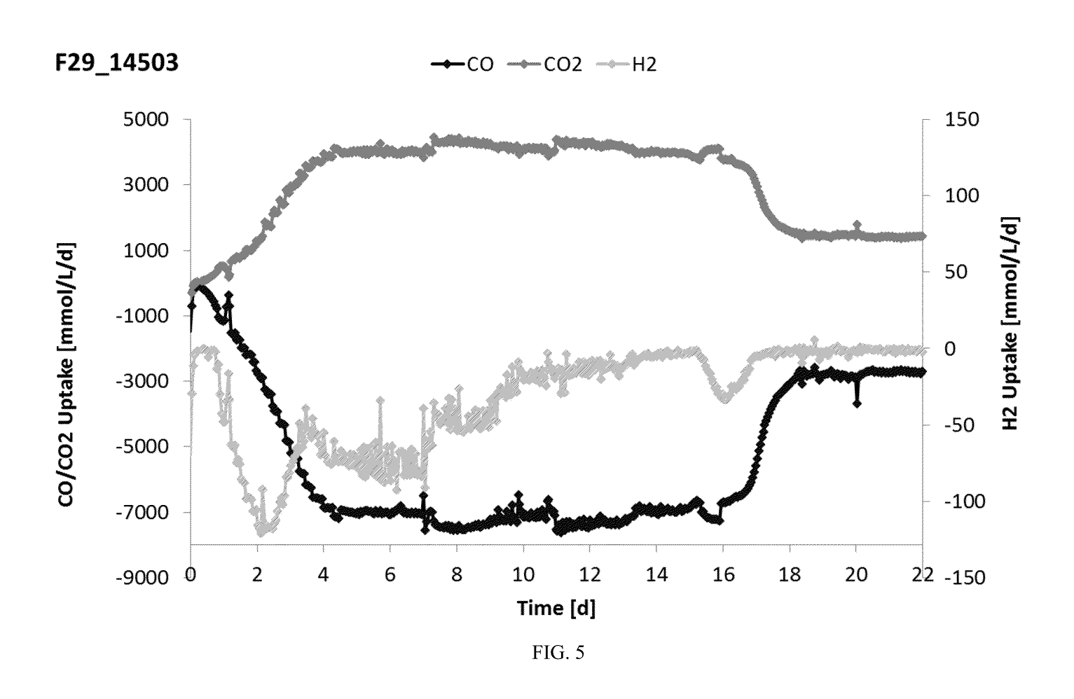

The results from the continuous culture are illustrated in FIG. 4, which shows metabolite (acetate, ethanol, and 2,3-butanediol) data at increasing 2-HIBA concentrations, and FIG. 5, which shows the gas (CO, CO₂, and H₂) uptake data at increasing 2-HIBA concentrations. During selection in continuous fermentation, the 2-HIBA concentration tolerated by the C. autoethanogenum LZ1561 culture was reproducibly raised from 1.1 g/L to 6.7 g/L (600% increase in tolerance) by slowly increasing the 2-HIBA concentration over a period of 85 days. Additional experiments suggest that this increased tolerance to 2-HIBA is not only the result of phenotypic adaptation, but of a genetic change in the strain.

Strain Validation

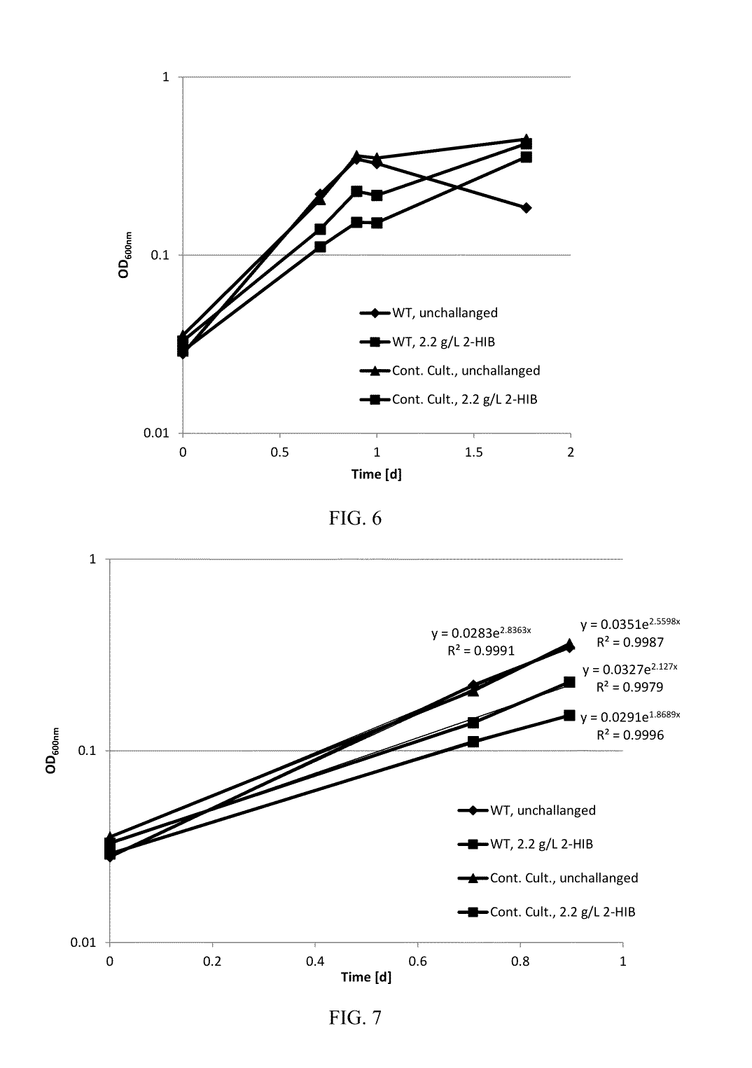

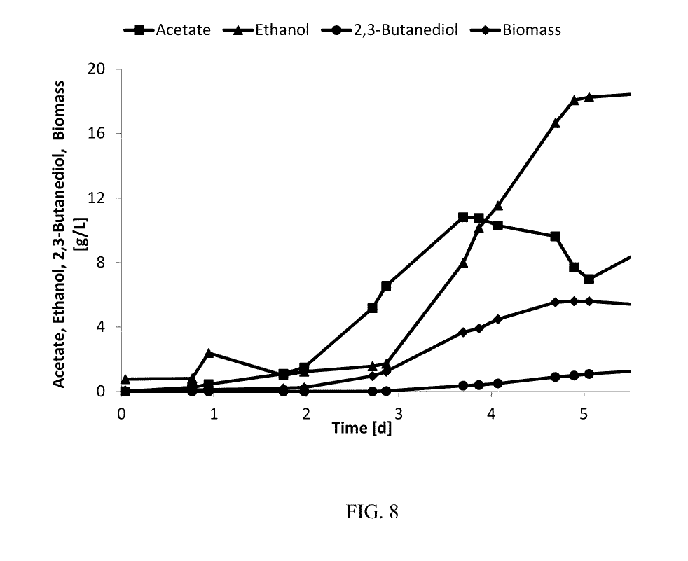

The selected strain from the continuous fermentation experiment shows an increased growth rate over unadapted C. autoethanogenum LZ1561. FIG. 6 shows growth profile with of C. autoethanogenum LZ1561 and 2-HIBA tolerant strain with and without 2.2 g/L 2-HIBA challenge. Calculated growth rates indicate a 25% increased growth rate of the 2-HIBA tolerant strain over C. autoethanogenum LZ1561 when challenged with 2.2 g/L 2-HIBA, as illustrated in FIG. 7.

### Example 4

This example describes the metabolic profile of a butyric acid (2-HIBA) tolerant strain, particularly the production of 2,3-butanediol by the 2-HIBA tolerant strain compared to C. autoethanogenum LZ1561.

The 2-HIBA tolerant strain was cultured in a 2 L BioFlo 115 system (New Brunswick Scientific Corp., Edison, N.J.) with a working volume of ˜1.5 L. The CSTR system was equipped with two six-bladed Rushton impellers and baffles to enhance gas to liquid mass transfer and mixing, which is an important element in ensuring a controlled reactor environment. The temperature of the fermenter was maintained at 37° C. A pH and an oxidation-reduction potential (ORP) electrode (Broadley-James Corporation) were inserted through the headplate and their readings recorded at 5 min intervals. The pH of the culture was maintain at 5.3 using a peristaltic pump that was connected the fermenter and triggered as soon as the pH dropped below the set point to dose a 5 M NH₄OH solution into the fermenter. All gas and liquid lines connected to the fermenter were made of gas impermeable tubing to minimize oxygen diffusion through the tube walls. Mass flow controllers (MFCs) calibrated for the individual gases (N₂, CO, CO₂ and H₂) were used to allow precise mixing and flow control. A gas mixture of 3% H₂, 45% CO, 17% CO₂, and 35% N₂ was fed to the culture at the maximum flow rate of 167 mL of gas per L of liquid per min. The dilution rate of the fermenter or the bacteria growth rate was set to 1 day⁻¹.

Under continuous conditions, the 2-HIBA tolerant strain produced about 16-18 g/L ethanol and about 1.3 g/L 2,3-butanediol (BDO). Accordingly, the 2-HIBA tolerant strain demonstrates an ethanol:BDO production ratio of about 13.8:1 to about 12.3:1. In contrast, under similar conditions, C. autoethanogenum LZ1561 generally produces about 18 g/L ethanol and about 6 g/L BDO, for an ethanol:BDO production ratio of about 3:1. It appears, therefore, that the 2-HIBA tolerant strain produces less BDO than C. autoethanogenum LZ1561 and is, accordingly, characterized by a high ethanol:BDO production ratio.

### Example 5

This example describes nucleic acid and amino acid mutations observed in butyric acid (2-HIBA) tolerant strains.

The genetic basis of butyric acid tolerance in two butyric acid tolerant strains was investigated. The first strain was developed in continuous culture and the second strain was developed through selection on plates. Both strains were sequenced using Illumina Hi-Seq platform with a coverage >100×.

In both strains, several SNPs (single nucleotide polymorphisms) were found. The continuous culture strain had 17 SNPs and 5 indels (insertions or deletions), while the plated strain had 10 SNPs and 4 indels. Some of the SNPs were shared between both strains. Some SNPs resulted in proteins with synonymous (SYN) mutations and some SNPs resulted in proteins with non-synonymous (NON) mutations. These mutations are summarized in the following table:

| | Change in nucleic acid sequence (positive strand) | | | | Change in amino acid sequence | | |
|---|---|---|---|---|---|---|---|
| | | | Butyric | | | | Butyric |
| | | | acid | | | | acid |
| | | C. auto | tolerant | Genome | | C. auto | tolerant |
| Element | Type | LZ1561 | C. auto | position | Type | LZ1561 | C. auto |
| Promoter region for | INDEL | ATTTTT | ATTTTTT | 11895- | | | |
| microcompartment | | | | 11900 | | | |
| protein | | | | | | | |
| Electron transfer | SNP | G | A | 62332- | NON | A | V |
| flavoprotein | | | | 62332 | | | |
| alpha/beta-subunit | | | | | | | |
| Electron transfer | SNP | C | T | 63604- | NON | V | I |
| flavoprotein | | | | 63604 | | | |
| alpha/beta-subunit | | | | | | | |
| RNA-binding S4 | SNP | C | T | 192932- | SYN | G | G |
| domain protein | | | | 192932 | | | |
| Promoter region of | SNP | T | A | 470041- | | | |
| transcriptional | | | | 470041 | | | |
| regulator AbrB- | | | | | | | |
| family | | | | | | | |
| Peptide chain | SNP | T | C | 581122- | NON | V | A |
| release factor 2 | | | | 581122 | | | |
| Protein of unknown | INDEL | CGGG | CGGGG | 779093- | | | |
| function DUF2088 | | | | 779096 | | | |
| RNA polymerase, | SNP | T | C | 1215498- | SYN | S | S |
| sigma 70 subunit, | | | | 1215498 | | | |
| RpoD/SigA | | | | | | | |
| D-glucuronyl C5- | SNP | C | T | 1259008- | NON | G | E |
| epimerase domain | | | | 1259008 | | | |
| protein | | | | | | | |
| Promoter region of | SNP | C | A | 1265489- | | | |
| a cyclase family | | | | 1265489 | | | |
| protein | | | | | | | |
| Promoter region of | SNP | T | C | 1321002- | | | |
| response regulator | | | | 1321002 | | | |
| receiver | | | | | | | |
| Serine-type D-Ala- | SNP | T | G | 1528659- | SYN | V | V |
| D-Ala | | | | 1528659 | | | |
| carboxypeptidase | | | | | | | |
| histidine kinase | SNP | T | C | 1588074- | NON | Y | C |
| | | | | 1588074 | | | |
| Promoter region for | INDEL | TAAAAAAA | TAAAAAAAA | 1598710- | | | |
| microcompartment | | | | 1598717 | | | |
| protein | | | | | | | |
| Heat shock protein | SNP | A | G | 1862579- | SYN | L | L |
| DnaJ | | | | 1862579 | | | |
| Cell wall binding | INDEL | CAAAAAAAA | CAAAAAAA | 1896725- | | | |
| repeat 2-containing | | | | 1896733 | | | |
| protein | | | | | | | |
| ABC-type | INDEL | TAAAAAAA | TAAAAAA | 2159715- | | | |
| transporter, | | | | 2159722 | | | |
| periplasmic subunit | | | | | | | |
| protein of unknown | INDEL | GAAAAA | GAAAAAA | 2519826- | | | |
| function DUF6 | | | | 2519831 | | | |
| transmembrane | | | | | | | |
| transcriptional | SNP | A | G | 2568685- | NON | V | A |
| regulator, DeoR | | | | 2568685 | | | |
| family | | | | | | | |
| protein of unknown | INDEL | TCCCCCC | TCCCCCCCC | 2712839- | | | |
| function DUF917 | | | | 2712845 | | | |
| Adenine deaminase | SNP | G | A | 3161844- | SYN | C | C |
| | | | | 3161844 | | | |
| ATP-binding | SNP | C | A | 3440339- | NON | S | R |
| region ATPase | | | | 3440339 | | | |
| domain protein | | | | | | | |
| Promoter region of | SNP | A | G | 3483967- | | | |
| YheO-like domain- | | | | 3483967 | | | |
| containing protein | | | | | | | |
| Promoter region of | SNP | G | T | 3662659- | | | |
| hypothetical protein | | | | 3662659 | | | |
| Transcriptional | SNP | A | G | 3832515- | NON | I | T |
| regulator, PadR- | | | | 3832515 | | | |
| like family | | | | | | | |
| Intergenic region | INDEL | ACCCC | ACCCCC | 4063046- | | | |
| | | | | 4063050 | | | |
| Aspartyl/glutamyl- | SNP | A | G | 4071430- | NON | S | P |
| tRNA(Asn/Gln) | | | | 4071430 | | | |
| amidotransferase | | | | | | | |
| subunit B | | | | | | | |
| 3-isopropylmalate | SNP | C | T | 4344797- | NON | V | I |
| dehydrogenase | | | | 4344797 | | | |

The full sequences of each of these elements are provided, as described in the following table:

| SEQ ID NO | Type | Element | Strain |
|---|---|---|---|
| 1 | Nucleic | Promoter region for | C. autoethanogenum LZ1561 |
| | acid | microcompartment protein | |
| 2 | Nucleic | Promoter region for | Butyric acid tolerant C. autoethanogenum |
| | acid | microcompartment protein | |
| 3 | Nucleic | Microcompartment protein 1 | C. autoethanogenum LZ1561 |
| | acid | | Butyric acid tolerant C. autoethanogenum |
| 4 | Amino | Microcompartment protein 1 | C. autoethanogenum LZ1561 |
| | acid | | Butyric acid tolerant C. autoethanogenum |
| 5 | Nucleic | Electron transfer | C. autoethanogenum LZ1561 |
| | acid | flavoprotein beta-subunit | |
| 6 | Nucleic | Electron transfer | Butyric acid tolerant C. autoethanogenum |
| | acid | flavoprotein beta-subunit | |
| 7 | Amino | Electron transfer | C. autoethanogenum LZ1561 |
| | acid | flavoprotein beta-subunit | |
| 8 | Amino | Electron transfer | Butyric acid tolerant C. autoethanogenum |
| | acid | flavoprotein beta-subunit | |
| 9 | Nucleic | Electron transfer | C. autoethanogenum LZ1561 |
| | acid | flavoprotein alpha-subunit | |
| 10 | Nucleic | Electron transfer | Butyric acid tolerant C. autoethanogenum |
| | acid | flavoprotein alpha-subunit | |
| 11 | Amino | Electron transfer | C. autoethanogenum LZ1561 |
| | acid | flavoprotein alpha-subunit | |
| 12 | Amino | Electron transfer | Butyric acid tolerant C. autoethanogenum |
| | acid | flavoprotein alpha-subunit | |
| 13 | Nucleic | RNA-binding S4 domain | C. autoethanogenum LZ1561 |
| | acid | protein | |
| 14 | Nucleic | RNA-binding S4 domain | Butyric acid tolerant C. autoethanogenum |
| | acid | protein | |
| 15 | Amino | RNA-binding S4 domain | C. autoethanogenum LZ1561 |
| | acid | protein | Butyric acid tolerant C. autoethanogenum |
| 16 | Nucleic | Promoter region of | C. autoethanogenum LZ1561 |
| | acid | transcriptional regulator | |
| | | AbrB family | |
| 17 | Nucleic | Promoter region of | Butyric acid tolerant C. autoethanogenum |
| | acid | transcriptional regulator | |
| | | AbrB family | |
| 18 | Nucleic | transcriptional regulator | C. autoethanogenum LZ1561 |
| | acid | AbrB family | Butyric acid tolerant C. autoethanogenum |
| 19 | Amino | transcriptional regulator | C. autoethanogenum LZ1561 |
| | acid | AbrB family | Butyric acid tolerant C. autoethanogenum |
| 20 | Nucleic | Peptide chain release factor 2 | C. autoethanogenum LZ1561 |
| | acid | | |
| 21 | Nucleic | Peptide chain release factor 2 | Butyric acid tolerant C. autoethanogenum |
| | acid | | |
| 22 | Amino | Peptide chain release factor 2 | C. autoethanogenum LZ1561 |
| | acid | | |
| 23 | Amino | Peptide chain release factor 2 | Butyric acid tolerant C. autoethanogenum |
| | acid | | |
| 24 | Nucleic | Protein of unknown function | Butyric acid tolerant C. autoethanogenum |
| | acid | DUF2088 | |
| 25 | Nucleic | Protein of unknown function | Butyric acid tolerant C. autoethanogenum |
| | acid | DUF2089 | |
| 26 | Amino | Protein of unknown function | C. autoethanogenum LZ1561 |
| | acid | DUF2090 | |
| 27 | Amino | Protein of unknown function | Butyric acid tolerant C. autoethanogenum |
| | acid | DUF2091 | |
| 28 | Nucleic | RNA polymerase, sigma 70 | C. autoethanogenum LZ1561 |
| | acid | subunit, RpoD/SigA | |
| 29 | Nucleic | RNA polymerase, sigma 70 | Butyric acid tolerant C. autoethanogenum |
| | acid | subunit, RpoD/SigA | |
| 30 | Amino | RNA polymerase, sigma 70 | C. autoethanogenum LZ1561 |
| | acid | subunit, RpoD/SigA | Butyric acid tolerant C. autoethanogenum |
| 31 | Nucleic | D-glucuronyl C5-epimerase | C. autoethanogenum LZ1561 |
| | acid | domain protein | |
| 32 | Nucleic | D-glucuronyl C5-epimerase | Butyric acid tolerant C. autoethanogenum |
| | acid | domain protein | |
| 33 | Amino | D-glucuronyl C5-epimerase | C. autoethanogenum LZ1561 |
| | acid | domain protein | |
| 34 | Amino | D-glucuronyl C5-epimerase | Butyric acid tolerant C. autoethanogenum |
| | acid | domain protein | |
| 35 | Nucleic | Promoter region of a cyclase | C. autoethanogenum LZ1561 |
| | acid | family protein | |
| 36 | Nucleic | Promoter region of a cyclase | Butyric acid tolerant C. autoethanogenum |
| | acid | family protein | |
| 37 | Nucleic | cyclase family protein | C. autoethanogenum LZ1561 |
| | acid | | Butyric acid tolerant C. autoethanogenum |
| 38 | Amino | cyclase family protein | C. autoethanogenum LZ1561 |
| | acid | | Butyric acid tolerant C. autoethanogenum |
| 39 | Nucleic | Promoter region of response | C. autoethanogenum LZ1561 |
| | acid | regulator receiver | |
| 40 | Nucleic | Promoter region of response | Butyric acid tolerant C. autoethanogenum |
| | acid | regulator receiver | |
| 41 | Nucleic | Response regulator receiver | C. autoethanogenum LZ1561 |
| | acid | | Butyric acid tolerant C. autoethanogenum |
| 42 | Amino | Response regulator receiver | C. autoethanogenum LZ1561 |
| | acid | | Butyric acid tolerant C. autoethanogenum |
| 43 | Nucleic | Serine-type D-Ala-D-Ala | C. autoethanogenum LZ1561 |
| | acid | carboxypeptidase | |
| 44 | Nucleic | Serine-type D-Ala-D-Ala | Butyric acid tolerant C. autoethanogenum |
| | acid | carboxypeptidase | |
| 45 | Amino | Serine-type D-Ala-D-Ala | C. autoethanogenum LZ1561 |
| | acid | carboxypeptidase | Butyric acid tolerant C. autoethanogenum |
| 46 | Nucleic | Histidine kinase | C. autoethanogenum LZ1561 |
| | acid | | |
| 47 | Nucleic | Histidine kinase | Butyric acid tolerant C. autoethanogenum |
| | acid | | |
| 48 | Amino | Histidine kinase | C. autoethanogenum LZ1561 |
| | acid | | |
| 49 | Amino | Histidine kinase | Butyric acid tolerant C. autoethanogenum |
| | acid | | |
| 50 | Nucleic | promoter region for | C. autoethanogenum LZ1561 |
| | acid | microcompartment protein | |
| 51 | Nucleic | promoter region for | Butyric acid tolerant C. autoethanogenum |
| | acid | microcompartment protein | |
| 52 | Nucleic | microcompartment protein 2 | C. autoethanogenum LZ1561 |
| | acid | | Butyric acid tolerant C. autoethanogenum |
| 53 | Amino | microcompartment protein 2 | C. autoethanogenum LZ1561 |
| | acid | | Butyric acid tolerant C. autoethanogenum |
| 54 | Nucleic | Heat shock protein DnaJ | C. autoethanogenum LZ1561 |
| | acid | | |
| 55 | Nucleic | Heat shock protein DnaJ | Butyric acid tolerant C. autoethanogenum |
| | acid | | |
| 56 | Amino | Heat shock protein DnaJ | C. autoethanogenum LZ1561 |
| | acid | | Butyric acid tolerant C. autoethanogenum |
| 57 | Nucleic | Cell wall binding repeat 2- | C. autoethanogenum LZ1561 |
| | acid | containing protein | |
| 58 | Nucleic | Cell wall binding repeat 2- | Butyric acid tolerant C. autoethanogenum |
| | acid | containing protein | |
| 59 | Amino | Cell wall binding repeat 2- | C. autoethanogenum LZ1561 |
| | acid | containing protein | |
| 60 | Amino | Cell wall binding repeat 2- | Butyric acid tolerant C. autoethanogenum |
| | acid | containing protein | |
| 61 | Nucleic | ABC-type transporter, | C. autoethanogenum LZ1561 |
| | acid | periplasmic subunit | |
| 62 | Nucleic | ABC-type transporter, | Butyric acid tolerant C. autoethanogenum |
| | acid | periplasmic subunit | |
| 63 | Amino | ABC-type transporter, | C. autoethanogenum LZ1561 |
| | acid | periplasmic subunit | |
| 64 | Amino | ABC-type transporter, | Butyric acid tolerant C. autoethanogenum |
| | acid | periplasmic subunit | |
| 65 | Nucleic | protein of unknown function | C. autoethanogenum LZ1561 |
| | acid | DUF6 transmembrane | |
| 66 | Nucleic | protein of unknown function | Butyric acid tolerant C. autoethanogenum |
| | acid | DUF6 transmembrane | |
| 67 | Amino | protein of unknown function | C. autoethanogenum LZ1561 |
| | acid | DUF6 transmembrane | |
| 68 | Amino | protein of unknown function | Butyric acid tolerant C. autoethanogenum |
| | acid | DUF6 transmembrane | |
| 69 | Nucleic | transcriptional regulator, | C. autoethanogenum LZ1561 |
| | acid | DeoR family | |
| 70 | Nucleic | transcriptional regulator, | Butyric acid tolerant C. autoethanogenum |
| | acid | DeoR family | |
| 71 | Amino | transcriptional regulator, | C. autoethanogenum LZ1561 |
| | acid | DeoR family | |
| 72 | Amino | transcriptional regulator, | Butyric acid tolerant C. autoethanogenum |
| | acid | DeoR family | |
| 73 | Nucleic | protein of unknown function | C. autoethanogenum LZ1561 |
| | acid | DUF917 | |
| 74 | Nucleic | protein of unknown function | Butyric acid tolerant C. autoethanogenum |
| | acid | DUF917 | |
| 75 | Amino | protein of unknown function | C. autoethanogenum LZ1561 |
| | acid | DUF917 | |
| 76 | Amino | protein of unknown function | Butyric acid tolerant C. autoethanogenum |
| | acid | DUF917 | |
| 77 | Nucleic | Adenine deaminase | C. autoethanogenum LZ1561 |
| | acid | | |
| 78 | Nucleic | Adenine deaminase | Butyric acid tolerant C. autoethanogenum |
| | acid | | |
| 79 | Amino | Adenine deaminase | C. autoethanogenum LZ1561 |
| | acid | | Butyric acid tolerant C. autoethanogenum |
| 80 | Nucleic | ATP-binding region ATPase | C. autoethanogenum LZ1561 |
| | acid | domain protein | |
| 81 | Nucleic | ATP-binding region ATPase | Butyric acid tolerant C. autoethanogenum |
| | acid | domain protein | |
| 82 | Amino | ATP-binding region ATPase | C. autoethanogenum LZ1561 |
| | acid | domain protein | |
| 83 | Amino | ATP-binding region ATPase | Butyric acid tolerant C. autoethanogenum |
| | acid | domain protein | |
| 84 | Nucleic | Promoter region of YheO- | C. autoethanogenum LZ1561 |
| | acid | like domain-containing | |
| | protein | | |
| 85 | Nucleic | Promoter region of YheO- | Butyric acid tolerant C. autoethanogenum |
| | acid | like domain-containing | |
| | protein | | |
| 86 | Nucleic | YheO-like domain- | C. autoethanogenum LZ1561 |
| | acid | containing protein | Butyric acid tolerant C. autoethanogenum |
| 87 | Amino | YheO-like domain- | C. autoethanogenum LZ1561 |
| | acid | containing protein | Butyric acid tolerant C. autoethanogenum |
| 88 | Nucleic | Promoter region of | C. autoethanogenum LZ1561 |
| | acid | hypothetical protein | |
| 89 | Nucleic | Promoter region of | Butyric acid tolerant C. autoethanogenum |
| | acid | hypothetical protein | |
| 90 | Nucleic | hypothetical protein | C. autoethanogenum LZ1561 |
| | acid | | Butyric acid tolerant C. autoethanogenum |
| 91 | Amino | hypothetical protein | C. autoethanogenum LZ1561 |
| | acid | | Butyric acid tolerant C. autoethanogenum |
| 92 | Nucleic | Transcriptional regulator, | C. autoethanogenum LZ1561 |
| | acid | PadR-like family | |
| 93 | Nucleic | Transcriptional regulator, | Butyric acid tolerant C. autoethanogenum |
| | acid | PadR-like family | |
| 94 | Amino | Transcriptional regulator, | C. autoethanogenum LZ1561 |
| | acid | PadR-like family | |
| 95 | Amino | Transcriptional regulator, | Butyric acid tolerant C. autoethanogenum |
| | acid | PadR-like family | |
| 96 | Nucleic | Aspartyl/glutamyl- | C. autoethanogenum LZ1561 |
| | acid | tRNA(Asn/Gln) | |
| | | amidotransferase subunit B | |
| 97 | Nucleic | Aspartyl/glutamyl- | Butyric acid tolerant C. autoethanogenum |
| | acid | tRNA(Asn/Gln) | |
| | | amidotransferase subunit B | |
| 98 | Amino | Aspartyl/glutamyl- | C. autoethanogenum LZ1561 |
| | acid | tRNA(Asn/Gln) | |
| | | amidotransferase subunit B | |
| 99 | Amino | Aspartyl/glutamyl- | Butyric acid tolerant C. autoethanogenum |
| | acid | tRNA(Asn/Gln) | |
| | | amidotransferase subunit B | |
| 100 | Nucleic | 3-isopropylmalate | C. autoethanogenum LZ1561 |
| | acid | dehydrogenase | |
| 101 | Nucleic | 3-isopropylmalate | Butyric acid tolerant C. autoethanogenum |
| | acid | dehydrogenase | |
| 102 | Amino | 3-isopropylmalate | C. autoethanogenum LZ1561 |
| | acid | dehydrogenase | |
| 103 | Amino | 3-isopropylmalate | Butyric acid tolerant C. autoethanogenum |
| | acid | dehydrogenase | |

In summary, the butyric acid tolerant strain may comprise one or more mutations in any of the aforementioned genes, genetic elements, or proteins. In particular, the butyric acid tolerant strain may comprise one or more nucleic acid sequences of SEQ ID NOs: 2, 6, 10, 14, 17, 21, 24, 25, 29, 32, 36, 40, 44, 47, 51, 55, 58, 62, 66, 70, 74, 78, 81, 85, 89, 93, 97, and 101 or one or more amino acid sequences of SEQ ID NOs: 8, 12, 23, 27, 34, 49, 60, 64, 68, 72, 76, 83, 95, 99, and 103.

While not wishing to be bound by any particular theory, the inventors have attempted to explain how these mutations may affect butyric acid tolerance.

Chaperones or heat shock proteins, such as GroESL or DnaKJ, are known to improve tolerance to certain stressors. For example, heat shock proteins are described as improving tolerance to butanol in Clostridium acetobutylicum (Tomas, Appl Environmen Microbiol, 69: 4951-4965, 2003; Zingaro, Metab Eng, 16: 196-205, 2013; Zingaro, MBio, 3: e00308-12, 2012). A mutation in DnaKJ may lead to improved tolerance to stressors and enhanced tolerance to butyric acid.

Bacterial microcompartments are organelles composed entirely of protein. They promote specific metabolic processes by encapsulating and co-localizing enzymes with their substrates and cofactors, protecting vulnerable enzymes in a defined microenvironment and by sequestering toxic or volatile intermediates (Yeates, Curr Opin Struct Biol, 21: 223-231, 2011). A change in promoter regions of two different of such microcompartment proteins (on two different loci on the genome) may have led to upregulation of microcompartment formation which may contributes to enhanced butyric acid tolerance.

DD-transpeptidases, such as serine-type D-Ala-D-Ala carboxypeptidase, cross-links peptidoglycan chains to form rigid cell walls in Gram-positive bacteria such as Clostridia. The structure and fluidity of the cell wall is known to influence the tolerance of bacteria to stressors, such as butanol in Clostridium acetobutylicum (Baer, Appl Environ Microbiol, 53: 2854-2861, 1987). A mutation in the may affect membrane fluidity and enhance butyric acid tolerance. The mutation in D-Ala-D-Ala carboxypeptidase did not change the protein sequence, but rather affected codon usage and, potentially, translation.

Sigma 70 is the primary sigma factor during exponential growth. A change in the sequence of rpoD will have a global impact on gene expression and may contribute to improved tolerance to butyric acids. The mutation in rpoD did not change the protein sequence, but rather affected codon usage and, potentially, translation. In addition, two global regulators in the DeoR and PadR family contained SNPs, resulting in amino acid changes. DeoR transcriptional regulators are known to control transporters mostly as repressors, while PadR transcriptional regulators are known to control the expression of genes associated with detoxification, such as efflux pumps, which could be the reason for the improved butyric acid tolerance.

In both strains, SNPs were also found associated with ABC transport systems that may have a detoxifying effect on butyric acids. In addition, a mutation in the ATP-binding region ATPase domain protein has been observed. Both ATP requiring systems may be important for the energy metabolism of the cells, which in turn is important for tolerance and metabolite production rates, such as production of ethanol and 2,3-butanediol.

AbrB-type family proteins are multipass membrane proteins involved in the regulation of alkylation and other cell damage (Daley, Science, 308: 1321-1323, 2005). A change in the promoter region of a transcriptional regulator of such an AbrB-type family protein could enhance butyric acid tolerance.

Mutations were also found in sensor and signaling elements. A mutation in the promoter region of a response regulator receiver may also result in a global effect (affecting, e.g., transcription factors) that leads to enhanced butyric acid tolerance or a changed metabolic profile to favor production acetyl-CoA derived products, such as ethanol, over pyruvate-derived products, such as 2,3-butanediol.

Electron-transfer proteins play an important role in energy metabolism (Köpke, PNAS USA, 107: 13087-13092, 2010). One of five pairs of electron transfer flavoproteins was found to be altered, with a non-synonymous amino acid change in each subunit. This mutation may have altered and possibly improved electron flow, allowing the microorganism to better cope with high butyric acid concentrations. It may also have altered bacterial metabolism to favor production acetyl-CoA derived products, such as ethanol, over pyruvate-derived products, such as 2,3-butanediol.

Two genes involved in amino acid metabolism, an aspartyl/glutamyl-tRNA amidotransferase and a 3-isopropylmalate dehydrogenase, contained a SNP resulting in an amino acid change. In E. coli and Salmonella, butyric acids such as 2-hydroxyisobutyric acid have been reported to inhibit branched-chain amino acid biosynthesis pathways, such as the ketol-acid reductoisomerase enzyme (Arfin, J Biol Chem, 244: 1118-1127, 1969; Chunduru, Biochem, 28: 486-493, 1989; Mrachko, Arch Biochem Biophys, 294: 446-453, 1992). The change in the 3-isopropylmalate dehydrogenase therefore may result in enhanced tolerance against butyric acids and protect against competitive or feedback inhibition. Amino acid production may also be altered by this change and potentially also production of metabolites that use similar precursors, such as 2,3-butanediol (Köpke, Appl Environ Microbiol, 77: 5467-5475, 2011). The change in aspartyl/glutamyl-tRNA amidotransferase may impact the pool of arginine and glutamate amino acids. Amino acids such as glutamate or arginine are known to be involved in acid resistance (Foster, Nature Rev Microbiol, 2: 898-907, 2004) and likely improve butyric acid tolerance.

In addition, mutations were found in genes with hypothetical functions that may be involved in tolerance or product formation. In one case, a mutation at the end of a gene for a protein of unknown function DUF917 resulted in a frameshift that leads to a fusion with a second gene for a protein of unknown function DUF917, thus resulting in a fusion-protein with potentially altered functionality.

All references, including publications, patent applications, and patents, cited herein are hereby incorporated by reference to the same extent as if each reference were individually and specifically indicated to be incorporated by reference and were set forth in its entirety herein. The reference to any prior art in this specification is not, and should not be taken as, an acknowledgement that that prior art forms part of the common general knowledge in the field of endeavour in any country.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A bacterium that tolerates at least 2.5 g/L of butyric acid.

2. The bacterium of claim 1, wherein the bacterium tolerates at least 5 g/L of butyric acid.

3. The bacterium of claim 1, wherein the bacterium tolerates at least 6.7 g/L of butyric acid.

4. The bacterium of claim 1, wherein the bacterium tolerates at least 10 g/L of butyric acid.

5. The bacterium of claim 1, wherein the bacterium is derived from a parental bacterium that cannot tolerate at least 2.5 g/L of butyric acid.

6. The bacterium of claim 1, wherein the butyric acid is 2-hydroxyisobutyric acid (2-HIBA).

7. The bacterium of claim 1, wherein the bacterium comprises one or more nucleic acid sequences selected from the group consisting of SEQ ID NOs: 2, 6, 10, 14, 17, 21, 24, 25, 29, 32, 36, 40, 44, 47, 51, 55, 58, 62, 66, 70, 74, 78, 81, 85, 89, 93, 97, and 101.

8. The bacterium of claim 1, wherein the bacterium comprises one or more amino sequences selected from the group consisting of SEQ ID NOs: 8, 12, 23, 27, 34, 49, 60, 64, 68, 72, 76, 83, 95, 99, and 103.

9. The bacterium of claim 1, wherein the bacterium produces one or more products selected from the group consisting of ethanol, acetate, and 2,3-butanediol.

10. The bacterium of claim 1, wherein the bacterium is a carboxydotrophic bacterium.

11. The bacterium of claim 1, wherein the bacterium is derived from a bacterium selected from genus Clostridium, Moorella, Oxobacter, Peptostreptococcus, Acetobacterium, Eubacterium, or Butyribacterium.

12. The bacterium of claim 1, wherein the bacterium is derived from Clostridium autoethanogenum, Clostridium ljungdahli, Clostridium carboxidivorans, Clostridium drakei, Clostridium scatologenes, Clostridium aceticum, Clostrdium formicoaceticum, Clostridium magnum, Butyribacterium methyotrphoicum, Acetbacterium woodii, Alkalibaculum bacchi, Blautia producta, Eubacterium limosum, Moorella thermoacetica, Sporomusa ovate, Sporomusa silvacetica, Sporomusa sphaeroides, Oxobacter pfennigii, or Thermoanaerbacter kiuvi.

13. The bacterium of claim 1, wherein the bacterium is derived from Clostridium autoethanogenum or Clostridium ljungdahlii.

14. The bacterium of claim 1, wherein the bacterium is derived from Clostridium autoethanogenum deposited under DSMZ accession number DSM23693.

15. A method of producing a product, comprising culturing the bacterium of claim 1 in the presence of a substrate whereby the bacterium produces a product.

16. The method of claim 15, wherein the product is selected from the group consisting of ethanol, acetate, and 2,3-butanediol.

17. The method of claim 15, wherein the substrate comprises one or more of CO, CO₂, and H₂.
